(19) United States
(12) Patent Application Publication
(10) Pub. No.: US 2022/0023358 A1
(43) Pub. Date: Jan. 27, 2022

(54) COMBINATION THERAPIES OF MICROORGANISMS AND IMMUNE MODULATORS FOR USE IN TREATING CANCER

(71) Applicant: Synlogic Operating Company, Inc., Cambridge, MA (US)

(72) Inventors: Jose M. Lora, Boston, MA (US); Anna Sokolovska, Somerville, MA (US)

(21) Appl. No.: 17/291,313

(22) PCT Filed: Nov. 8, 2019
(86) PCT No.: PCT/US2019/060406

§ 371 Date: May 5, 2021

Related U.S. Application Data
(60) Provisional application No. 62/848,294, filed on May 15, 2019.
(60) Provisional application No. 62/757,452, filed on Nov. 8, 2018.

(51) Int. Cl. A61K 35/741; A61P 35/00; A61K 38/46; A61K 31/7084; A61K 31/198; A61K 31/513; A61K 45/06

(52) U.S. Cl. CPC A61K 35/741; A61P 35/00; A61K 38/46; C12Y 307/01003; A61K 31/198; A61K 31/513; A61K 45/06; A61K 31/7084

(57) ABSTRACT

Combination therapies comprising a microorganism and an immune modulator, and methods of modulating and treating cancers are disclosed.

### RELATED APPLICATIONS

The instant application claims priority to U.S. Provisional Application No. 62/757,452, filed on Nov. 8, 2018, and U.S. Provisional Application No. 62/848,294, filed on May 15, 2019, the entire contents of each of which are expressly incorporated herein by reference.

### SEQUENCE LISTING

The instant application contains a Sequence Listing which has been submitted electronically in ASCII format and is hereby incorporated by reference in its entirety. Said ASCII copy, created on Mar. 15, 2019, is named 126046-05020_ST25.txt and is 1,784 kilobytes in size.

### BACKGROUND

Current cancer therapies typically employ the use of immunotherapy, surgery, chemotherapy, radiation therapy, or some combination thereof (American Cancer Society). While these drugs have shown great benefits to cancer patients, many cancers remain difficult to treat using conventional therapies. Currently, many conventional cancer therapies are administered systemically and adversely affect healthy tissues, resulting in significant side effects. For example, many cancer therapies focus on activating the immune system to boost the patient's anti-tumor response (Kong et al., 2014). However, despite such therapies, the microenvironment surrounding tumors remains highly immune suppressive. In addition, systemic altered immunoregulation provokes immune dysfunction, including the onset of opportunistic autoimmune disorders and immune-related adverse events.

Major efforts have been made over the past few decades to develop cytotoxic drugs that specifically target cancer cells. In recent years there has been a paradigm shift in oncology in which the clinical problem of cancer is considered not only to be the accumulation of genetic abnormalities in cancer cells but also the tolerance of these abnormal cells by the immune system. Consequently, recent anti-cancer therapies have been designed specifically to target the immune system rather than cancer cells. Such therapies aim to reverse the cancer immunotolerance and stimulate an effective antitumor immune response. For example, current immunotherapies include immunostimulatory molecules that are pattern recognition receptor (PRR) agonists or immunostimulatory monoclonal antibodies that target various immune cell populations that infiltrate the tumor microenvironment. However, despite their immune-targeted design, these therapies have been developed clinically as if they were conventional anticancer drugs, relying on systemic administration of the immunotherapeutic (e.g., intravenous infusions every 2-3 weeks). As a result, many current immunotherapies suffer from toxicity due to a high dosage requirement and also often result in an undesired autoimmune response or other immune-related adverse events.

Thus, there is an unmet need for effective cancer therapies that are able to target poorly vascularized, hypoxic tumor regions specifically target cancerous cells, while minimally affecting normal tissues and boost the immune systems to fight the tumors, including avoiding or reversing the cancer immunotolerance.

### SUMMARY

The present disclosure provides compositions, methods, and uses of a bacterium that selectively targets tumors and tumor cells in combination with one or more immune modulator(s), e.g., immune initiators and/or immune sustainers. The bacterium described herein is a wild-type bacterium, e.g., a probiotic bacterium, or a bacterial chassis which does not comprise a non-native immune modulator gene and/or express a non-native immune modulator protein or molecule. In some embodiments, however, a bacterial chassis may comprise one or more auxotrophies, an antibiotic resistance cassette, and/or a deletion of an endogenous phage. The use of wild-type bacterium and bacterial chassis in combination with at least one immune modulator is safe and provides targeted and local delivery of therapeutic compositions. The bacteria described herein surprisingly bolster the effect of the immune modulator, as compared to administration of an immune modulator alone, or the administration of the bacteria, alone. In some embodiments, the effect is synergistic. For example, the induction of an immune response is synergistically enhanced by the phagocytosis of the bacterium, as discussed in more detail herein.

In one aspect, disclosed herein is a pharmaceutical composition comprising an isolated bacterium, at least one immune modulator, and a pharmaceutically acceptable carrier, wherein the bacterium is a wild-type bacterium or a bacterial chassis.

In one embodiment, the at least one immune modulator is at least one immune initiator. In one embodiment, the immune initiator is capable of enhancing oncolysis, activating antigen presenting cells (APCs), and/or priming and activating T cells. In one embodiment, the immune initiator is a STING agonist, arginine, 5-FU, TNFα, IFNγ, IFNβ1, agonistic anti-CD40 antibody, CD40L, SIRPα, GMCSF, agonistic anti-OXO40 antibody, OXO40L, agonistic anti-4-1BB antibody, 4-1BBL, agonistic anti-GITR antibody, GITRL, anti-PD1 antibody, anti-PDL1 antibody, or azurin. In one aspect disclosed herein is a pharmaceutical composition, wherein the STING agonist is c-diAMP, c-GAMP, or c-diGMP. In one embodiment, the immune initiator is a cytokine, a chemokine, a single chain antibody, a ligand, a metabolic converter, a T cell co-stimulatory receptor, a T cell co-stimulatory receptor ligand, an engineered chemotherapy, or a lytic peptide. In one embodiment, the immune initiator is arginine. In one embodiment, the immune initiator is 5-FU.

In one embodiment, the at least one immune modulator is at least one immune sustainer. In one embodiment, the immune sustainer is capable of enhancing trafficking and infiltration of T cells, enhancing recognition of cancer cells by T cells, enhancing effector T cell response, and/or overcoming immune suppression. In one embodiment, the immune sustainer is a metabolic converter, arginine, a STING agonist, CXCL9, CXCL10, anti-PD1 antibody, anti-PDL1 antibody, anti-CTLA4 antibody, agonistic anti-GITR antibody or GITRL, agonistic anti-OX40 antibody or OX40L, agonistic anti-4-1BB antibody or 4-1BBL, IL-15, IL-15 sushi, IFNγ, or IL-12. In one embodiment, the immune sustainer is a cytokine, a chemokine, a single chain antibody, a ligand, a metabolic converter, a T cell co-stimulatory receptor, or a T cell co-stimulatory receptor ligand. In one embodiment, the at least one immune sustainer is a kynureninase. In one embodiment, the immune sustainer is arginine In one embodiment, the immune sustainer is a STING agonist. In one embodiment, the STING agonist is c-diAMP, c-GAMP, or c-diGMP.

In one embodiment, the at least one immune modulator comprises at least one immune initiator and at least one immune sustainer. In one embodiment, the at least one immune modulator is not produced by the bacterium.

In one embodiment, the bacterium is a wild-type E. coli Nissle bacterium.

In one embodiment, the bacterial chassis is a bacterium comprising at least one mutation or deletion in a gene which results in one or more auxotrophies. In one embodiment, the bacterial chassis is a bacterium comprising a thyA auxotrophy and/or a dapA auxotrophy. In one embodiment, the bacterial chassis is an E. coli, Lactobacillus, Lactococcus, Salmonella, Listeria, Lactobacillus, Lactococcus, Bifida bacterium, C. novyi, Streptococcus pyogenes, Myco bovis, or Klebsiella bacterium. In one embodiment, the bacterial chassis further comprises a phage deletion.

In one embodiment, the pharmaceutical composition is formulated for intratumoral administration. In one embodiment, the composition comprises an isolated bacterium, at least one immune modulator, and a pharmaceutically acceptable carrier, wherein the bacterium is a wild-type bacterium or a bacterial chassis.

In one aspect, disclosed herein is a syringe comprising a pharmaceutical composition, wherein the composition comprises an isolated bacterium, at least one immune modulator, and a pharmaceutically acceptable carrier, wherein the bacterium is a wild-type bacterium or a bacterial chassis.

In one aspect, disclosed herein is a kit comprising a pharmaceutical composition, wherein the composition comprises an isolated bacterium, at least one immune modulator, and a pharmaceutically acceptable carrier, wherein the bacterium is a wild-type bacterium or a bacterial chassis, and instructions for use thereof.

In one aspect disclosed herein is a kit comprising a syringe comprising a pharmaceutical composition, wherein the composition comprises an isolated bacterium, at least one immune modulator, and a pharmaceutically acceptable carrier, wherein the bacterium is a wild-type bacterium or a bacterial chassis, and instructions for use thereof.

In one aspect disclosed herein is a kit comprising i) a first composition comprising an isolated bacterium, wherein the bacterium is a wild-type bacterium or a bacterial chassis, ii) a second composition comprising an immune modulator, and iii) instructions for use thereof. In one embodiment, the first composition is a lyophilized composition. In one embodiment, the instructions for use indicate that the first composition is for administration to a subject prior to the second composition; the second composition is for administration to a subject prior to the first composition; or the first and second compositions are combined before administration to a subject.

In one aspect disclosed herein is a method of treating cancer in a subject, the method comprising administering to the subject a pharmaceutical composition, wherein the composition comprises an isolated bacterium, at least one immune modulator, and a pharmaceutically acceptable carrier, wherein the bacterium is a wild-type bacterium or a bacterial chassis, thereby treating cancer in the subject. In one embodiment, the administering is intratumoral injection. In one embodiment, the method further comprises a step of selecting a subject who would benefit from treatment with the bacterium and the at least one immune modulator. In one embodiment, the bacterium colonizes a tumor in the subject. In one embodiment, the administering is not oral administration. In one embodiment, the bacterium comprises a homogenous population of predefined bacteria. In one embodiment, the homogenous population of predefined bacteria comprise E. coli Nissle.

In one aspect disclosed herein is a method of inducing and sustaining an immune response in a subject, the method comprising administering to the subject a pharmaceutical composition, wherein the composition comprises an isolated bacterium, at least one immune modulator, and a pharmaceutically acceptable carrier, wherein the bacterium is a wild-type bacterium or a bacterial chassis, thereby inducing and sustaining the immune response in the subject. In one embodiment, the administering is intratumoral injection. In one embodiment, the method further comprises a step of selecting a subject who would benefit from treatment with the bacterium and the at least one immune modulator. In one embodiment, the bacterium colonizes a tumor in the subject. In one embodiment, the administering is not oral administration. In one embodiment, the bacterium comprise a homogenous population of predefined bacteria. In one embodiment, the homogenous population of predefined bacteria comprise E. coli Nissle.

In one aspect, disclosed herein is a method of inducing an abscopal effect in a subject having a tumor, the method comprising administering to the subject a pharmaceutical composition, wherein the composition comprises an isolated bacterium, at least one immune modulator, and a pharmaceutically acceptable carrier, wherein the bacterium is a wild-type bacterium or a bacterial chassis, thereby inducing the abscopal effect in the subject. In one embodiment, the administering is intratumoral injection. In one embodiment, the method further comprises a step of selecting a subject who would benefit from treatment with the bacterium and the at least one immune modulator. In one embodiment, the bacterium colonizes a tumor in the subject. In one embodiment, the administering is not oral administration. In one embodiment, the bacterium comprises a homogenous population of predefined bacteria. In one embodiment, the homogenous population of predefined bacteria comprise E. coli Nissle.

In one aspect, disclosed herein is a method of inducing immunological memory in a subject having a tumor, the method comprising administering to the subject a pharmaceutical composition, wherein the composition comprises an isolated bacterium, at least one immune modulator, and a pharmaceutically acceptable carrier, wherein the bacterium is a wild-type bacterium or a bacterial chassis, thereby inducing the immunological memory in the subject. In one embodiment, the administering is intratumoral injection. In one embodiment, the method further comprises a step of selecting a subject who would benefit from treatment with the bacterium and the at least one immune modulator. In one embodiment, the bacterium colonizes a tumor in the subject. In one embodiment, the administering is not oral administration. In one embodiment, the bacterium comprises a homogenous population of predefined bacteria. In one embodiment, the homogenous population of predefined bacteria comprise E. coli Nissle.

In one aspect disclosed herein is a method of inducing partial regression of a tumor in a subject, the method comprising administering to the subject a pharmaceutical composition, wherein the composition comprises an isolated bacterium, at least one immune modulator, and a pharmaceutically acceptable carrier, wherein the bacterium is a wild-type bacterium or a bacterial chassis, thereby inducing partial regression of the tumor in the subject. In one embodiment, the partial regression is a decrease in size of the tumor by at least about 10%, at least about 25%, at least about 50%, or at least about 75%. In one embodiment, the administering is intratumoral injection. In one embodiment, the method further comprises a step of selecting a subject who would benefit from treatment with the bacterium and the at least one immune modulator. In one embodiment, the bacterium colonizes a tumor in the subject. In one embodiment, the administering is not oral administration. In one embodiment, the bacterium comprise a homogenous population of predefined bacteria. In one embodiment, the homogenous population of predefined bacteria comprise E. coli Nissle.

In one aspect disclosed herein is a method of inducing complete regression of a tumor in a subject, the method comprising administering to the subject a pharmaceutical composition, wherein the composition comprises an isolated bacterium, at least one immune modulator, and a pharmaceutically acceptable carrier, wherein the bacterium is a wild-type bacterium or a bacterial chassis, thereby inducing complete regression of the tumor in the subject. In one embodiment, the tumor is not detectable in the subject after administration of the pharmaceutically acceptable composition. In one embodiment, the administering is intratumoral injection. In one embodiment, the method further comprises a step of selecting a subject who would benefit from treatment with the bacterium and the at least one immune modulator. In one embodiment, the bacterium colonizes a tumor in the subject. In one aspect disclosed herein is a method of inducing complete regression of a tumor in a subject, wherein the administering is not oral administration. In one embodiment, the bacterium comprise a homogenous population of predefined bacteria. In one embodiment, the homogenous population of predefined bacteria comprise E. coli Nissle.

In one aspect, disclosed herein is a method of treating cancer in a subject, the method comprising administering a bacterium to the subject, wherein the bacterium is a wild-type bacterium or a bacterial chassis; and administering at least one immune modulator to the subject, thereby treating cancer in the subject. In one embodiment, the administering steps are performed at the same time; administering of the bacterium to the subject occurs before administering of the at least one immune modulator to the subject; or administering of the at least one immune modulator to the subject occurs before administering of the bacterium to the subject. In one embodiment, the method further comprises a step of selecting a subject who would benefit from treatment with the bacterium and the at least one immune modulator. In one embodiment, the bacterium colonizes a tumor in the subject. In one embodiment, the administering of the bacterium is intratumoral injection. In one embodiment, the administering is not oral administration. In one embodiment, the administering of the at least one immune modulator is an intravenous injection. In one embodiment, the bacterium comprise a homogenous population of predefined bacteria. In one embodiment, the homogenous population of predefined bacteria comprise E. coli Nissle. In one embodiment, the at least one immune modulator comprises at least one immune initiator and at least one immune sustainer. In one embodiment, the at least one immune initiator is selected from the immune initiators listed in Table 5, and the at least one immune sustainer is selected from the immune sustainers listed in Table 6. In one embodiment, the at least one immune initiator is a STING agonist, and the at least one immune sustainer is a kynureninase.

In one aspect, disclosed herein is a method of inducing and sustaining an immune response in a subject, the method comprising administering a bacterium to the subject, wherein the bacterium is a wild-type bacterium or a bacterial chassis; and administering at least one immune modulator to the subject, thereby inducing and sustaining the immune response in the subject. In one embodiment, administering of the bacterium to the subject occurs before administering of the at least one immune modulator to the subject; or administering of the at least one immune modulator to the subject occurs before administering of the bacterium to the subject. In one embodiment, the method further comprises a step of selecting a subject who would benefit from treatment with the bacterium and the at least one immune modulator. In one embodiment, the bacterium colonizes a tumor in the subject. In one embodiment, administering of the bacterium is intratumoral injection. In one embodiment, the administering is not oral administration. In one embodiment, the administering of the at least one immune modulator is intravenous injection. In one embodiment, the bacterium comprise a homogenous population of predefined bacteria. In one embodiment, the homogenous population of predefined bacteria comprise E. coli Nissle. In one embodiment, the at least one immune modulator comprises at least one immune initiator and at least one immune sustainer. In one embodiment, the at least one immune initiator is selected from the immune initiators listed in Table 5, and the at least one immune sustainer is selected from the immune sustainers listed in Table 6. In one embodiment, the at least one immune initiator is a STING agonist, and the at least one immune sustainer is a kynureninase.

In certain aspects, the microorganism is a bacteria, e.g., Salmonella typhimurium, Escherichia coli Nissle, Clostridium novyi NT, and Clostridium butyricum miyairi, as well as other exemplary bacterial strains provided herein. In some embodiments, the bacteria are able to selectively home to tumor microenvironments. Thus, in certain embodiments, the microorganisms are administered systemically, e.g., via oral administration, intravenous injection, subcutaneous injection, intra tumor injection or other means, and are able to selectively colonize a tumor site.

In another aspect, disclosed herein is a composition comprising an immune initiator, e.g., a cytokine, chemokine, single chain antibody, ligand, metabolic converter, T cell co-stimulatory receptor, T cell co-stimulatory receptor ligand, engineered chemotherapy, or lytic peptide; and a microorganism. In yet another aspect, disclosed herein is a composition comprising an immune sustainer, e.g., a chemokine, a cytokine, a single chain antibody, a ligand, a metabolic converter, a T cell co-stimulatory receptor, or a T cell co-stimulatory receptor ligand; and a first microorganism.

In one embodiment, the immune initiator is capable of enhancing oncolysis, activating antigen presenting cells (APCs), and/or priming and activating T cells. In another embodiment, the immune initiator is capable of enhancing oncolysis. In another embodiment, the immune initiator is capable of activating APCs. In yet another embodiment, the immune initiator is capable of priming and activating T cells.

In one embodiment, the immune initiator is a therapeutic molecule. In one embodiment, the immune imitator is a nucleic acid molecule that mediates RNA interference, microRNA response or inhibition, TLR response, antisense gene regulation, target protein binding, or gene editing. In one embodiment, the immune imitator is a cytokine, a chemokine, a single chain antibody, a ligand, a metabolic converter, a T cell co-stimulatory receptor, a T cell co-stimulatory receptor ligand, an engineered chemotherapy, or a lytic peptide. [30] In one embodiment, the immune initiator is a STING agonist, arginine, 5-FU, TNFα, IFNy, IFNβ1, agonistic anti-CD40 antibody, CD40L, SIRPα, GMCSF, agonistic anti-OXO40 antibody, OXO40L, agonistic anti-4-1BB antibody, 4-1BBL, agonistic anti-GITR antibody, GITRL, anti-PD1 antibody, anti-PDL1 antibody, or azurin. In one embodiment, the immune initiator is a STING agonist.

In one embodiment, the immune initiator is arginine. In one embodiment, the immune initiator is 5-FU. In one embodiment, the immune initiator is TNFα. In one embodiment, the immune initiator is IFNy. In one embodiment, the immune initiator is IFNβ1. In one embodiment, the immune initiator is an agonistic anti-CD40 antibody. In one embodiment, the immune initiator is SIRPα. In one embodiment, the immune initiator is CD40L. In one embodiment, the immune initiator is GMCSF. In one embodiment, the immune initiator is an agonistic anti-OXO40 antibody. In another embodiment, the immune initiator is OXO40L. In one embodiment, the immune initiator is an agonistic anti-4-IBB antibody. In one embodiment, the immune initiator is 4-1BBL. In one embodiment, the immune initiator is an agonistic anti-GITR antibody. In another embodiment, the immune initiator is GITRL. In one embodiment, the immune initiator is an anti-PD1 antibody. In one embodiment, the immune initiator is an anti-PDL1 antibody. In one embodiment, the immune initiator is azurin. In one embodiment, the immune initiator is arginine. In one embodiment, the immune initiator is 5-FU.

In one embodiment, the immune initiator is a STING agonist. In one embodiment, the STING agonist is c-diAMP. In one embodiment, the STING agonist is c-GAMP. In one embodiment, the STING agonist is c-diGMP.

In one embodiment, the immune sustainer is capable of enhancing trafficking and infiltration of T cells, enhancing recognition of cancer cells by T cells, enhancing effector T cell response, and/or overcoming immune suppression. In one embodiment, the immune sustainer is capable of enhancing trafficking and infiltration of T cells. In one embodiment, the immune sustainer is capable of enhancing recognition of cancer cells by T cells. In one embodiment, the immune sustainer is capable of enhancing effector T cell response. In one embodiment, the immune sustainer is capable of overcoming immune suppression.

In one embodiment, the immune sustainer is a therapeutic molecule. In one embodiment, the immune sustainer is a nucleic acid molecule that mediates RNA interference, microRNA response or inhibition, TLR response, antisense gene regulation, target protein binding, or gene editing.

In one embodiment, the immune sustainer is a cytokine, a chemokine, a single chain antibody, a ligand, a metabolic converter, a T cell co-stimulatory receptor, a T cell co-stimulatory receptor ligand, or a secreted or displayed peptide.

In one embodiment, the immune sustainer is a metabolic converter, arginine, a STING agonist, CXCL9, CXCL10, anti-PD1 antibody, anti-PDL1 antibody, anti-CTLA4 antibody, agonistic anti-GITR antibody or GITRL, agonistic anti-OX40 antibody or OX40L, agonistic anti-4-1BB antibody or 4-1BBL, IL-15, IL-15 sushi, IFNγ, or IL-12. In one embodiment, the immune sustainer is arginine.

In one embodiment, the immune sustainer is a STING agonist. In one embodiment, the STING agonist is c-diAMP, c-GAMP, or c-diGMP. In one embodiment, the STING agonist is c-diAMP. In one embodiment, the STING agonist is c-GAMP. In one embodiment, the STING agonist is c-diGMP.

In one embodiment, the immune initiator is not the same as the immune sustainer. In one embodiment, the immune initiator is different than the immune sustainer.

In one embodiment, the bacterium is an auxotroph in a gene that is not complemented when the bacterium is present in a tumor. In one embodiment, the gene that is not complemented when the bacterium is present in a tumor is a dapA gene. In one embodiment, the bacterium is an auxotroph in a gene that is complemented when the bacterium is present in a tumor. In one embodiment, the gene that is complemented when the bacterium is present in a tumor is a thyA gene.

In one embodiment, the bacterium further comprises a mutation or deletion in an endogenous prophage.

In one embodiment, the bacterium is non-pathogenic. In one embodiment, the bacterium is Escherichia coli Nissle.

In another aspect, the immune modulator is a dimerized IL-12, comprising a p35 IL-12 subunit gene sequence linked to a p40 IL-12 subunit gene sequence by a linker sequence, with or without a secretion tag sequence. In one embodiment, the secretion tag sequence is selected from the group consisting of SEQ ID NO: 1235, 1146-1154, 1156, and 1168. In one embodiment, the linker sequence comprises SEQ ID NO: 1194. In one embodiment, the p35 IL-12 subunit gene sequence comprises SEQ ID NO: 1192, and wherein the p40 IL-12 subunit gene sequence comprises SEQ ID NO: 1193. In one embodiment, the gene sequence comprises a sequence selected from the group consisting of SEQ ID NOs: 1169-1179.

In another aspect, the immune modulator is an IL-15 fusion protein, such as an IL-15 gene sequence fused to a sushi domain sequence. In one embodiment, the sequence is selected from the group consisting of SEQ ID NOs: 1195-1198.

In one embodiment, the microorganism disclosed herein is a bacterium. In one embodiment, the microorganism disclosed herein is a yeast. In one embodiment, the microorganism is an E. coli bacterium. In one embodiment, the microorganism is an E. coli Nissle bacterium.

In one embodiment, the microorganism disclosed herein comprises at least one mutation or deletion in a gene which results in one or more auxotrophies. In one embodiment, the at least one deletion or mutation is in a dapA gene and/or a thyA gene.

In one embodiment, the microorganism disclosed herein comprises a phage deletion.

In one embodiment, the immune initiator is not arginine, TNFα, IFNγ, IFNβ1, GMCSF, anti-CD40 antibody, CD40L, agonistic anti-OX40 antibody, OXO40L, agonistic anti-41BB antibody , 41BBL, agonistic anti-GITR antibody, GITRL, anti-PD1 antibody, anti-PDL1 antibody, and/or azurin. In one embodiment, the immune initiator is not arginine. In one embodiment, the immune initiator is not TNFα. In one embodiment, the immune initiator is not IFNγ. In one embodiment, the immune initiator is not IFNβ1. In one embodiment, the immune initiator is not an anti-CD40 antibody. In one embodiment, the immune initiator is not CD40L. In one embodiment, the immune initiator is not GMCSF. In one embodiment, the immune initiator is not an agonistic anti-OXO40 antibody. In one embodiment, the immune initiator is not OXO40L. In one embodiment, the immune initiator is not an agonistic anti-4-1BB antibody. In one embodiment, the immune initiator is not 4-1BBL. In one embodiment, the immune initiator is not an agonistic anti-GITR antibody. In one embodiment, the immune initiator is not GITRL. In one embodiment, the immune initiator is not an anti-PD1 antibody. In one embodiment, the immune initiator is not an anti-PDL1 antibody. In one embodiment, the immune initiator is not azurin.

In one embodiment, the immune sustainer is not at least one enzyme of a kynurenine consumption pathway, at least one enzyme of an adenosine consumption pathway, anti-PD1 antibody, anti-PDL1 antibody, anti-CTLA4 antibody, IL-15, IL-15 sushi, IFNγ, agonistic anti-GITR antibody, GITRL, an agonistic anti-OX40 antibody, OX40L, an agonistic anti-4-1BB antibody, 4-1BBL, or IL-12. In one embodiment, at least enzyme of the adenosine consumption pathway is selected from add, xapA, deoD, xdhA, xdhB, and xdhC. In one embodiment, the immune sustainer is not at least one enzyme of a kynurenine consumption pathway. In one embodiment, the immune sustainer is not at least one enzyme of an adenosine consumption pathway. In one embodiment, the immune sustainer is not arginine. In one embodiment, the immune sustainer is not at least one enzyme of an arginine biosynthetic pathway. In one embodiment, the immune sustainer is not an anti-PD1 antibody. In one embodiment, the immune sustainer is not an anti-PDL1 antibody. In one embodiment, the immune sustainer is not an anti-CTLA4 antibody. In one embodiment, the immune sustainer is not an agonistic anti-GITR antibody. In one embodiment, the immune sustainer is not GITRL. In one embodiment, the immune sustainer is not IL-15. In one embodiment, the immune sustainer is not IL-15 sushi. In one embodiment, the immune sustainer is not IFNγ. In one embodiment, the immune sustainer is not an agonistic anti-OX40 antibody. In one embodiment, the immune sustainer is not OX40L. In one embodiment, the immune sustainer is not an agonistic anti-4-1BB antibody. In one embodiment, the immune sustainer is not 4-1BBL. In one embodiment, the immune sustainer is not IL-12.

Exemplary nucleic acid sequences for use in constructing single-chain anti-CTLA-4 antibodies are described in International Patent Application PCT/US2017/013072, filed Jan. 11, 2017, published as WO2017/123675, the contents of which is herein incorporated by reference in its entirety. IN one embodiment, the immune modulator comprises a polypeptide comprising a sequence selected from SEQ ID NO: 765, SEQ ID NO: 766, SEQ ID NO: 767, SEQ ID NO: 768, SEQ ID NO: 769, SEQ ID NO: 770, SEQ ID NO: 771, SEQ ID NO: 772, SEQ ID NO: 773, SEQ ID NO: 774, SEQ ID NO: 775, and/or SEQ ID NO: 776.. In yet another embodiment, the polypeptide consists of a sequence selected from SEQ ID NO: 765, SEQ ID NO: 766, SEQ ID NO: 767, SEQ ID NO: 768, SEQ ID NO: 769, SEQ ID NO: 770, SEQ ID NO: 771, SEQ ID NO: 772, SEQ ID NO: 773, SEQ ID NO: 774, SEQ ID NO: 775, and/or SEQ ID NO: 776.

In one aspect, disclosed herein is a pharmaceutically acceptable composition comprising a microorganism disclosed herein, one or more immune modulators, and a pharmaceutically acceptable carrier. In one aspect, disclosed herein is a pharmaceutically acceptable composition comprising a composition disclosed herein, and a pharmaceutically acceptable carrier. In one embodiment, the composition is formulated for intratumoral injection. In another embodiment, the pharmaceutically acceptable composition is for use in treating a subject having caner. In another embodiment, the pharmaceutically acceptable composition is for use in inducing and modulating an immune response in a subject.

In one aspect, disclosed herein is a kit comprising a pharmaceutically acceptable composition disclosed herein, and instructions for use thereof.

In one aspect, disclosed herein is a method of treating cancer in a subject, the method comprising administering to the subject a pharmaceutically acceptable composition disclosed herein, thereby treating cancer in the subject.

In one aspect, disclosed herein is a method of inducing and sustaining an immune response in a subject, the method comprising administering to the subject a pharmaceutically acceptable composition disclosed herein, thereby inducing and sustaining the immune response in the subject.

In one aspect, disclosed herein is a method of inducing and sustaining an immune response in a subject, the method comprising administering to the subject a pharmaceutically acceptable composition described herein, thereby inducing and sustaining the immune response in the subject.

In another aspect, disclosed herein is a method of inducing an abscopal effect in a subject having a tumor, the method comprising administering to the subject a pharmaceutically acceptable composition described herein, thereby inducing the abscopal effect in the subject.

In one aspect, disclosed herein is a method of inducing immunological memory in a subject having a tumor, the method comprising administering to the subject a pharmaceutically acceptable composition described herein, thereby inducing the immunological memory in the subject.

In one aspect, disclosed herein is a method of inducing partial regression of a tumor in a subject, the method comprising administering to the subject a pharmaceutically acceptable composition described herein, thereby inducing the partial regression of the tumor in the subject. In one embodiment, the partial regression is a decrease in size of the tumor by at least about 10%, at least about 25%, at least about 50%, or at least about 75%.

In one aspect, disclosed herein is a method of inducing complete regression of a tumor in a subject, the method comprising administering to the subject a pharmaceutically acceptable composition described herein, thereby inducing the complete regression of the tumor in the subject. In one embodiment, the tumor is not detectable in the subject after administration of the pharmaceutically acceptable composition.

In one aspect, disclosed herein is a method of treating cancer in a subject, the method comprising administering a first microorganism to the subject, and administering an immune modulator, e.g., an immune sustainer and/or an immune initiator, to the subject, thereby treating cancer in the subject.

In one aspect, disclosed herein is a method of inducing and sustaining an immune response in a subject, the method comprising administering a first microorganism to the subject, and administering an immune modulator, e.g., an immune sustainer and/or an immune initiator, to the subject, thereby inducing and sustaining the immune response in the subject.

In one embodiment, the administering steps are performed at the same time. In one embodiment, the administering of the first microorganism to the subject occurs before the administering of the immune modulator to the subject. In one embodiment, the administering of the immune modulator to the subject occurs before the administering of the first microorganism to the subject.

In one aspect, disclosed herein is a method of treating cancer in a subject, the method comprising administering a first microorganism to the subject, and administering an immune modulator, e.g., an immune sustainer and/or an immune initiator, to the subject, thereby treating cancer in the subject.

In one aspect, disclosed herein is a method of inducing and sustaining an immune response in a subject, the method comprising administering a first microorganism to the subject, and administering an immune modulator, e.g., an immune sustainer and/or an immune initiator, to the subject, thereby inducing and sustaining the immune response in the subject.

In one embodiment, the administering is intratumoral injection.

Accordingly, the disclosure provides compositions comprising one or more bacteria and one or more immune modulators. In some embodiments, the immune modulator is an immune initiator, which may for example modulate, e.g., promote tumor lysis, antigen presentation by dendritic cells or macrophages, or T cell activation or priming. Examples of such immune initiators include cytokines or chemokines, such as TNFα, IFN-gamma and IFN-betal, a single chain antibody, such as anti-CD40 antibodies, or (3) ligands such as SIRPα or CD40L, a metabolic enzymes (biosynthetic or catabolic), such as a STING agonist producing enzyme, or (5) cytotoxic chemotherapies.

In some embodiments, the immune modulator is one or more STING agonist(s), such as c-di-AMP, 3′3′-cGAMP and/or c-2′3′-cGAMP.

In some embodiments, the immune modulator is a co-stimulatory receptor, including but not limited to OX40, GITR, 41BB.

In some embodiments, the composition further comprises one or more immune sustainers, which may modulate, e.g., enhance, tumor infiltration or the T cell response or modulate, e.g., alleviate, immune suppression. Such a sustainer may be selected from a cytokine or chemokine, a single chain antibody antagonistic peptide or ligand, and a metabolic enzyme pathways.

Examples of immune sustaining cytokines include IL-15 and CXCL10, which may be secreted into the tumor microenvironment. Non-limiting examples of single chain antibodies include anti-PD-1, anti-PD-L1, or anti-CTLA-4.

In some embodiments, the composition comprises bacteria that are auxotrophs for a particular metabolite, e.g., the bacterium is an auxotroph in a gene that is not complemented when the microorganism(s) is present in the tumor. In some embodiments, the bacterium is an auxotroph in the DapA gene. In some embodiments, the composition comprises bacteria that are auxotrophs for a particular metabolite, e.g., the bacterium is an auxotroph in a gene that is complemented when the microorganism(s) is present in the tumor. In some embodiments, the bacterium is an auxotroph in the ThyA gene. In some embodiments, the bacterium is an auxotroph in the TrpE gene.

In some embodiments, the bacterium is a Gram-positive bacterium. In some embodiments, the bacterium is a Gram-negative bacterium. In some embodiments, the bacterium is an obligate anaerobic bacterium. In some embodiments, the bacterium is a facultative anaerobic bacterium. Non-limiting examples of bacteria contemplated in the disclosure include Clostridium novyi NT, and Clostridium butyricum, and Bifidobacterium longum. In some embodiments, the bacterium is selected from E. coli Nissle, and E. coli K-12.

In some embodiments, the bacterium comprises an antibiotic resistance gene sequence.

Additionally, pharmaceutical compositions are provided, further comprising one or more immune checkpoint inhibitors, such as CTLA-4 inhibitor, a PD-1 inhibitor, and a PD-L1 inhibitor. Such checkpoint inhibitors may be administered in combination, sequentially or concurrently with the bacteria.

Additionally, pharmaceutical compositions are provided, further comprising one or more agonists of co-stimulatory receptors, such as OX40, GITR, and/or 41BB, including but not limited to agonistic molecules, such as ligands or agonistic antibodies which are capable of binding to co-stimulatory receptors, such as OX40, GITR, and/or 41BB. Such agonistic molecules may be administered in combination, sequentially or concurrently with the bacteria.

In any of these embodiments, a combination of bacteria can be used in conjunction with conventional cancer therapies, such as surgery, chemotherapy, targeted therapies, radiation therapy, tomotherapy, immunotherapy, cancer vaccines, hormone therapy, hyperthermia, stem cell transplant (peripheral blood, bone marrow, and cord blood transplants), photodynamic therapy, therapy, and blood product donation and transfusion, and oncolytic viruses. In any of these embodiments, the bacteria can be used in conjunction with a cancer or tumor vaccine.

### BRIEF DESCRIPTION OF THE FIGURES

FIG. 1 depicts a schematic showing the study design for localization of luminescent bacteria (SYN5353) in B16-F10 tumors in mice models.

FIG. 2 depicts wild type E. coli Nissle colonization in B16-F10 tumors. A luminescent bacteria (SYN-BioLum, 50μL) was injected into mice and specific colonization and spreading of the Nissle bacteria are shown throughout the tumor after 24 hours post injection.

FIG. 3A depicts the interstitial pressure forces after intratumoral injections of the wild-type E. coli Nissle into the subcutaneous space around the B16-F10 tumors. The pressures were measured 20-30 minutes after initial injection. FIG. 3B depicts the infiltration and specific colonization of the wild-type E. coli Nissle 72 hours post injection. FIG. 3C depicts the specific colonization of the wild-type E. coli Nissle 7 days and 15 days post injection.

FIG. 4A depicts bacterial abundance within dose and tumor homogenates at 72 hours post-intratumoral (i.t.) injection with ˜1×10⁶ CFU of EcN from various tumor models as measured by colony forming unit (CFU) assay. FIG. 4B depicts bacterial abundance measured by CFU (left axis) and relative bioluminescent units (RLU) (right axis) from CT26 tumors at the indicated time points post-i.t. injection with 1×10⁶ CFU of EcN-LuxABCDE compared to saline injected controls. FIG. 4C depicts bacterial abundance within tumor homogenates (filled in circle) or blood (hollow circle) for CT26 tumor-bearing mice treated with 1×10⁶ or 1×10⁷ CFUs EcN.

FIG. 5 depicts in vivo activity of SYNB (comprising a bacterial chassis: a wild-type E. coli Nissle strain containing dual auxotrophies for diaminopimelic acid and thymidine, and deletion of an endogenous phage) and SYNB1891 (comprising the SYNB Nissle strain plus an FNR-inducible dacA from Listeria monocytogenes integrated into the genome to produce the STING agonist ci-di-AMP) and average tumor growth in B16.F10 tumors. Mice were injected with the SYNB and SYNB1891 strains and average tumor growth was measured for 21 days.

FIGS. 6A, 6B, and 6C depict individual tumor growth of the SYNB (FIG. 6B) and SYNB1891 (FIG. 6C) strains post-injection in relation to the saline control (FIG. 6A).

FIGS. 7A, 7B, and 7C depict phagocytosis of SYNB1891-gfp by BMDC. BMDCs were incubated with pre-induced SYNB1891-gfp (MOI: 25) for lh in control media (FIGS. 7A and 7B) or pre-treated for 1 h with Cytochalasin D (10 μM) before bacterial incubation (FIG. 7C).

FIGS. 7D-7J depict mRNA levels in RAW 264.7 macrophages or WT BMDCs treated with Control EcN (MOI: 25), pre-induced SYN1891 (MOI: 25), LPS (100 ng/mL) or smSTING agonist (5 μg/mL) for 4 hours and demonstrates phagocytosis-dependent and STING-dependent Type I IFN production as well as the effect of chassis-derived LPS in the production of cytokines by SYNB1891. In indicated groups cells were pre-treated with for lh with Cytochalasin D (10 μM). Macrophages or BMDCs incubated in media alone served as a negative control. Cells were analyzed for the upregulation of Ifnbl and 116 mRNA (FIGS. 7D-7G). WT, TLR4^{-/-} and STING-^{-/-} BMDCs were treated with Control EcN (MOI: 25), pre-induced SYN1891 (MOI: 25), LPS (100 ng/mL), or smSTING agonist (5 μg/mL) for 4 hours. Cells were analyzed for the upregulation of Ifnb1, Il6 and Il12a mRNA (FIGS. 7H-7J). Data are representative of two or more independent experiments with mean and standard deviation shown. **P<0.005; ***P<0.0005; ****P<0.00001; two-tailed unpaired Student's t-test comparing the indicated groups.

FIG. 8 depicts a Mantel-Cox log-rank test for A20 tumor-bearing mice treated with either saline or 1×10⁹ CFUs SYNB1891-cmR (SYNB1891 containing a chloramphenicol resistance gene). CD4⁺ and CD8⁺ T cells were depleted via the i p administration of anti-CD4 or anti-CD8 antibodies 1 day before treatment initiation and twice weekly throughout the study with an isotype injected as control. Long-term survival is shown. Data are representative of two or more independent experiments.

FIG. 9 depicts the activation of the IRF reporter via luminescence (RLU) and fold IRF induction. THP-1 immortalized human monocyte cells containing an IRF-luciferase reporter and either the endogenous HAQ TMEM1 73 (STING) allele, knock-ins of the WT or R232H alleles or knockout of the TMEM1 73 gene were treated with pre-induced SYNB1891 (MOI: 100) or media alone overnight. BMDCs from each genotype individually incubated in media alone served as negative controls. Data are representative of two or more independent experiments with mean and standard deviation shown. *P<0.05; **P<0.005; ****P<0.00001; one-tailed unpaired Student's t-test comparing the indicated groups.

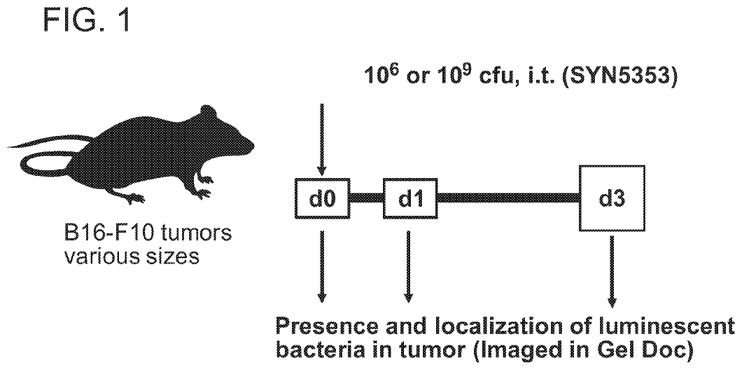

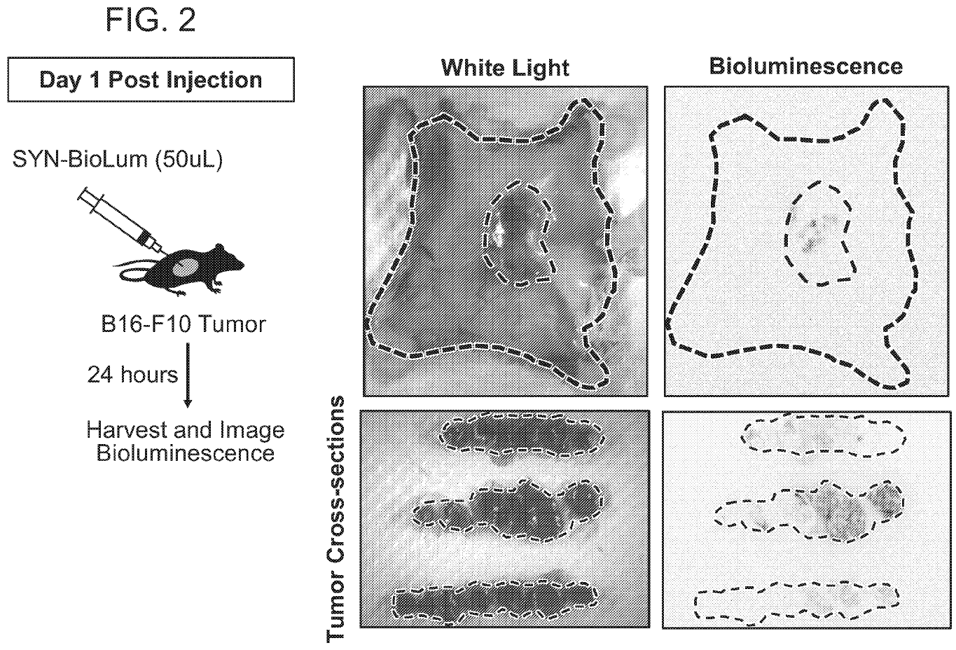

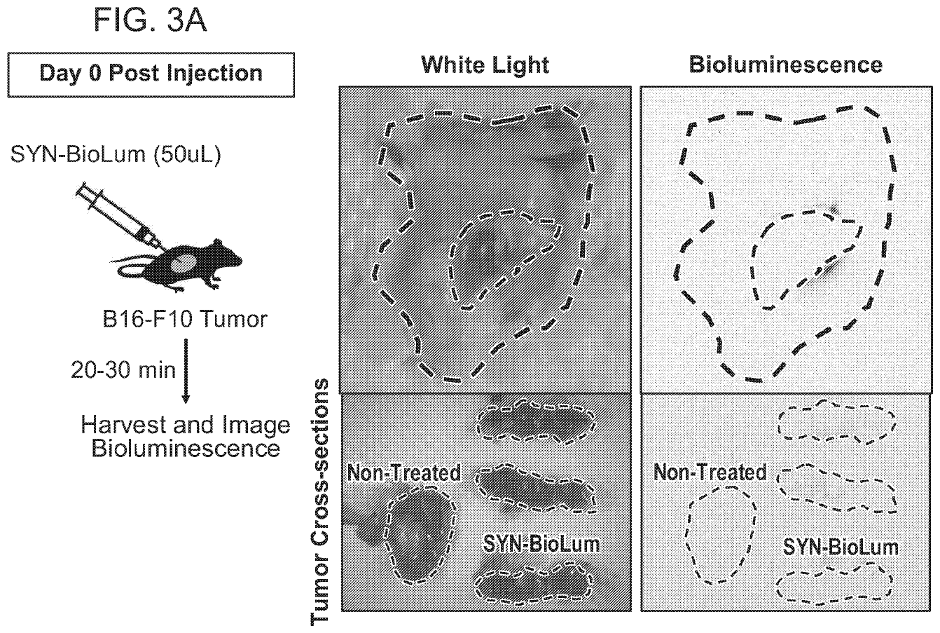

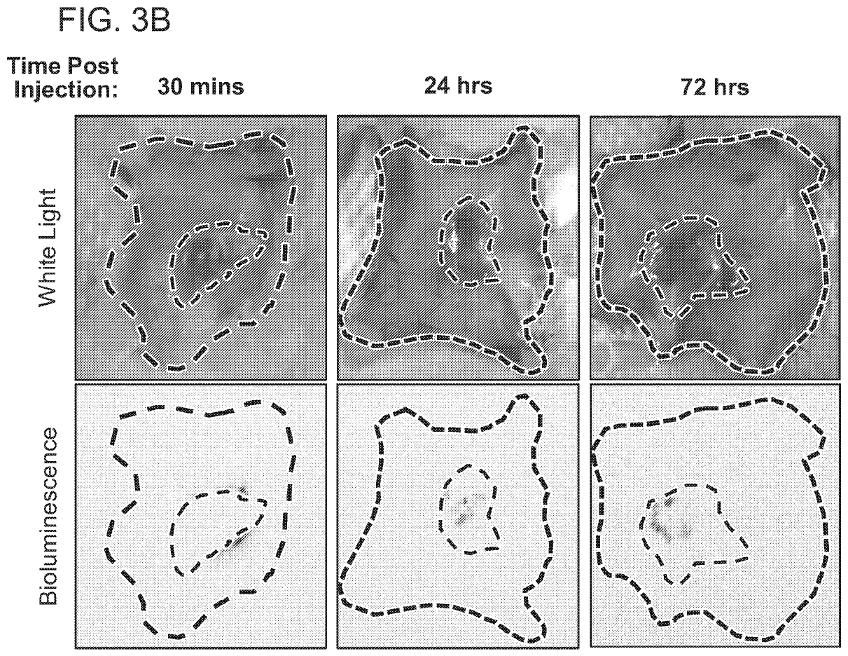

### DETAILED DESCRIPTION

Certain tumors are particularly difficult to manage using conventional therapies. Hypoxia is a characteristic feature of solid tumors, wherein cancerous cells are present at very low oxygen concentrations. Regions of hypoxia often surround necrotic tissues and develop as solid forms of cancer outgrow their vasculature. When the vascular supply is unable to meet the metabolic demands of the tumor, the tumor's microenvironment becomes oxygen deficient. Multiple areas within tumors contain <1% oxygen, compared to 3-15% oxygen in normal tissues (Vaupel and Hockel, 1995), and avascular regions may constitute 25-75% of the tumor mass (Dang et al., 2001). Approximately 95% of tumors are hypoxic to some degree (Huang et al., 2004). Systemically delivered anticancer agents rely on tumor vasculature for delivery, however, poor vascularization impedes the oxygen supply to rapidly dividing cells, rendering them less sensitive to therapeutics targeting cellular proliferation in poorly vascularized, hypoxic tumor regions. Radiotherapy fails to kill hypoxic cells because oxygen is a required effector of radiation-induced cell death. Hypoxic cells are up to three times more resistant to radiation therapy than cells with normal oxygen levels (Bettegowda et al., 2003; Tiecher, 1995; Wachsberger et al., 2003). For all of these reasons, nonresectable, locally advanced tumors are particularly difficult to manage using conventional therapies.

In addition to the challenges associated with targeting a hypoxic environment, therapies that specifically target and destroy cancers must recognize differences between normal and malignant tissues, including genetic alterations and pathophysiological changes that lead to heterogeneous masses with areas of hypoxia and necrosis.

The disclosure relates to combinations comprising one or more immune modulators, e.g., one or more immune initiators and/or one or more immune sustainers, and one or more microorganisms, e.g., bacteria, pharmaceutical compositions thereof, and methods of modulating or treating cancer. In certain embodiments, the bacteria are delivered locally to the tumor cells. In certain embodiments, the bacteria are capable of targeting cancerous cells. In certain embodiments, the bacteria are capable of targeting cancerous cells, particularly in low-oxygen conditions, such as in hypoxic tumor environments.

This disclosure relates to compositions and therapeutic methods for the local and tumor-specific delivery of immune modulators in order to treat cancers. In certain aspects, the disclosure relates to microorganisms that are capable of targeting cancerous cells in combination with one or more effector molecules e.g., immune modulators, such as any of the effector molecules provided herein. In contrast to existing conventional therapies, the hypoxic areas of tumors offer a perfect niche for the growth of anaerobic bacteria, the use of which offers an opportunity for eradication of advanced local tumors in a precise manner, sparing surrounding well-vascularized, normoxic tissue.

In some aspects, the disclosure provides delivery of a microorganism and one or more effector molecules, e.g., immune modulators, such as immune initiators and/or immune sustainers to tumor cells or the tumor microenvironment. In some aspects, the disclosure relates to a microorganism that is delivered locally, e.g., via local intra-tumoral administration, and one or more effector molecules, e.g., immune initiators and/or immune sustainers. In some aspects, the compositions and methods disclosed herein may be used to deliver one or more effector molecules, e.g., immune initiators and/or immune sustainers selectively to tumor cells, thereby reducing systemic cytotoxicity or systemic immune dysfunction, e.g., the onset of an autoimmune event or other immune-related adverse event.

In order that the disclosure may be more readily understood, certain terms are first defined. These definitions should be read in light of the remainder of the disclosure and as understood by a person of ordinary skill in the art. Unless defined otherwise, all technical and scientific terms used herein have the same meaning as commonly understood by a person of ordinary skill in the art. Additional definitions are set forth throughout the detailed description.

The generation of immunity to cancer is a potentially self-propagating cyclic process which has been referred to as the “Cancer-Immunity Cycle” (Chen and Mellman, Oncology Meets Immunology: The Cancer-Immunity Cycle; Immunity (2013) 39,:1-10), and which can lead to the broadening and amplification of the T cell response. The cycle is counteracted by inhibitory factors that lead to immune regulatory feedback mechanisms at various steps of the cycle and which can halt the development or limit the immunity.

The cycle essentially comprises a series of steps which need to occur for an anticancer immune response to be successfully mounted. The cycle includes steps, which must occur for the immune response to be initiated and a second series of events which must occur subsequently, in order for the immune response to be sustained (i.e., allowed to progress and expand and not dampened). These steps have been referred to as the “Cancer-Immunity Cycle” (Chen and Mellman, 2013), and are essentially as follows:

1. Release (oncolysis) and/or acquisition of tumor cell contents; Tumor cells break open and spill their contents, resulting in the release of neoantigens, which are taken up by antigen presenting cells (dendritic cells and macrophages for processing. Alternatively, antigen presenting cells may actively phagocytose tumors cells directly.

2. Activation of antigen presenting cells (APC) (dendritic cells and macrophages); In addition to the first step described above, the next step must involve release of proinflammatory cytokines or generation of proinflammatory cytokines as a result of release of DAMPs or PAMPs from the dying tumor cells to result in antigen presenting cell activation and subsequently an anticancer T cell response. Antigen presenting cell activation is critical to avoid peripheral tolerance to tumor derived antigens. If properly activated, antigen presenting cells present the previously internalized antigens on their surface in the context of MHCI and MHCII molecules alongside the proper co-stimulatory signals (CD80/86, cytokines, etc.) to prime and activate T cells.

3. Priming and Activation of T cells: Antigen presentation by DCs and macrophages causes the priming and activation of effector T cell responses against the cancer-specific antigens, which are seen as “foreign” by the immune system. This step is critical to the strength and breadth of the anti-cancer immune response, by determining quantity and quality of T effector cells and contribution of T regulatory cells. Additionally, proper priming of T cells can result in superior memory T cell formation and long lived immunity

4. Trafficking and Infiltration: Next, the activated effector T cells must traffic to the tumor and infiltrate the tumor.

5. Recognition of cancer cells by T cells and T cell support, and augmentation and expansion of effector T cell responses: Once arrived at the tumor site, the T cells can recognize and bind to cancer cells via their T cell receptors (TCR), which specifically bind to their cognate antigen presented within the context of MHC molecules on the cancer cells, and subsequently kill the target cancer cell. Killing of the cancer cell releases tumor associated antigens through lysis of tumor cells, and the cycle re-initiates, thereby increasing the volume of the response in subsequent rounds of the cycle. Antigen recognition by either MHC-I or MHC-II restricted T cells can result in additional effector functions, such as the release of chemokines and effector cytokines, further potentiating a robust antitumor response.

6. Overcoming immune suppression: Finally, overcoming certain deficiencies in the immune response to the cancer and/or overcoming the defense strategy of the cancer, i.e., overcoming the breaks that the cancer employs in fighting the immune response, can be viewed as another critical step in the cycle. In some cases, even though T cell priming and activation has occurred, other immunosuppressive cell subsets are actively recruited and activated to the tumor microenvironment, i.e., regulatory T cells or myeloid derived suppressor cells. In other cases, T cells may not receive the right signals to properly home to tumors or may be actively excluded from infiltrating the tumor. Finally, certain mechanisms in the tumor microenvironment exist, which are capable of suppressing or repressing the effector cells that are produced as a result of the cycle. Such resistance mechanisms co-opt immune-inhibitory pathways, often referred to as immune checkpoints, which normally mediate immune tolerance and mitigate cancer tissue damage (see e.g., Pardo11 (2012), The blockade of immune checkpoints in cancer immunotherapy; Nature Reviews Cancer volume 12, pages 252-264).

One important immune-checkpoint receptor is cytotoxic T-lymphocyte-associated antigen 4 (CTLA4), which down modulates the amplitude of T cell activation. Some immune-checkpoint receptors, such as programmed cell death protein 1 (PD1), limit T cell effector functions within tissues. By upregulating ligands for PD1, tumor cells and antigen presenting cells block antitumor immune responses in the tumor microenvironment. Multiple additional immune-checkpoint receptors and ligands, some of which are selectively upregulated in various types of tumor cells, are prime targets for blockade, particularly in combination with approaches that enhance the initiation or activation of antitumor immune responses.

Therapies have been developed to promote and support progression through the cancer-immunity cycle at one or more of the 6 steps. These therapies can be broadly classified as therapies that promote initiation of the immune response and therapies that help sustain the immune response.

As used herein the term “immune initiation” or “initiating the immune response” refers to advancement through the steps which lead to the generation and establishment of an immune response. For example, these steps could include the first three steps of the cancer immunity cycle described above, i.e., the process of antigen acquisition (step (1)), activation of dendritic cells and macrophages (step (2)), and/or the priming and activation of T cells (step (3)).

As used herein the term “immune sustenance” or “sustaining the immune response” refers to the advancement through steps which ensure the immune response is broadened and strengthened over time and which prevent dampening or suppression of the immune response. For example, these steps could include steps 4 through 6 of the cycle described, i.e., T cell trafficking and tumor infiltration, recognition of cancer cells though TCRs, and overcoming immune suppression, i.e., depletion or inhibition of T regulatory cells and preventing the establishment of other active suppression of the effector response.

Accordingly, in some embodiments, the compositions are capable of modulating, e.g., advancing the cancer immunity cycle by modulating, e.g., activating, promoting supporting, one or more of the steps in the cycle. In some embodiments, the compositions are capable of modulating, e.g., promoting, steps that modulate, e.g., intensify, the initiation of the immune response. In some embodiments, the compositions are capable of modulating, e.g., boosting, certain steps within the cycle that enhance sustenance of the immune response. In some embodiments, the compositions are capable of modulating, e.g., intensifying, the initiation of the immune response and modulating, e.g., enhancing, sustenance of the immune response.

Accordingly, in some embodiments, the one or more effector molecules, e.g., immune modulators, modulate, e.g., intensify the initiation of the immune response. Accordingly, in some embodiments, the one or more effector molecules, e.g., immune modulators, modulate, e.g., enhance, sustenance of the immune response. Accordingly, in some embodiments, the one or more effector molecules, e.g., immune modulators, modulate, e.g., intensify, the initiation of the immune response and the one or more one or more effector molecules, e.g., immune modulators, modulate, e.g., enhance, sustenance of the immune response.

An “effector”, “effector substance” or “effector molecule” refers to one or more molecules, therapeutic substances, or drugs of interest. In one embodiment, the “effector” is administered in combination with a microorganism, e.g., bacteria, and administered before, at the same time as, or after, the administration of the microorganism. In another embodiment, a microorganism described herein is administered in combination with at least two effectors and administered before, at the same time as, or after, the administration of the microorganism.

A non-limiting example of such effector or effector molecules are “immune modulators,” which include immune sustainers and/or immune initiators as described herein. In some embodiments, the composition comprises two or more effector molecules or immune modulators. In some embodiments, the composition comprises three, four, five, six, seven, eight, nine, or ten effector molecules or immune modulators. In some embodiments, the effector molecule or immune modulator is a therapeutic molecule that is useful for modulating or treating a cancer.

In some embodiments, the effector or immune modulator is a therapeutic molecule. In some embodiments, the effector molecule or immune modulator may be a nucleic acid molecule that mediates RNA interference, microRNA response or inhibition, TLR response, antisense gene regulation, target protein binding (aptamer or decoy oligos), or gene editing, such as CRISPR interference. Other types of effectors and immune modulators are described and listed herein.

Non-limiting examples of effector molecules and/or immune modulators include immune checkpoint inhibitors (e.g., CTLA-4 antibodies, PD-1 antibodies, PDL-1 antibodies), cytotoxic agents (e.g., Cly A, FASL, TRAIL, TNFα), immunostimulatory cytokines and co-stimulatory molecules (e.g., OX40 antibody or OX40L, CD28, ICOS, CCL21, IL-2, IL-18, IL-15, IL-12, IFN-gamma, IL-21, TNFs, GM-CSF), antigens and antibodies (e.g., tumor antigens, neoantigens, CtxB-PSA fusion protein, CPV-OmpA fusion protein, NY-ESO-1 tumor antigen, RAF1, antibodies against immune suppressor molecules, anti-VEGF, Anti-CXR4/CXCL12, anti-GLP1, anti-GLP2, anti-galectinl, anti-galectin3, anti-Tie2, anti-CD47, antibodies against immune checkpoints, antibodies against immunosuppressive cytokines and chemokines), DNA transfer vectors (e.g., endostatin, thrombospondin-1, TRAIL, SMAC, Stat3, Bc12, FLT3L, GM-CSF, IL-12, AFP, VEGFR2), and enzymes (e.g., E. coli CD, HSV-TK), immune stimulatory metabolites and biosynthetic pathway enzymes that produce them (STING agonists, e.g., c-di-AMP, 3′3′-cGAMP, and 2′3′-cGAMP; arginine, tryptophan).

Effectors may also include enzymes or other polypeptides (such as transporters or regulatory proteins) or other modifications (such as inactivation of certain endogenous genes, e.g., auxotrophies), which result in catabolism of immune suppressive or tumor growth promoting metabolites, such as kynurenine, adenosine and ammonia.

Immune modulators include, inter alia, immune initiators and immune sustainers.

As used herein, the term “immune initiator” or “initiator” refers to a class of effectors or molecules, e.g., immune modulators, or substances. Immune initiators may modulate, e.g., intensify or enhance, one or more steps of the cancer immunity cycle, including (1) lysis of tumor cells (oncolysis); (2) activation of APCs (dendritic cells and macrophages); and/or (3) priming and activation of T cells. In one embodiment, an immune initiator may be administered in combination with a microorganism of the disclosure. For example, a microorganism described herein is administered in combination with at least one immune initiator but administered before, at the same time as, or after, the administration of the microorganism. In other embodiments, a microorganism described herein is administered in combination with at least one immune initiator and at least one immune sustainer, but administered before, at the same time as, or after, the administration of the microorganism. Non-limiting examples of such immune initiators are described in further detail herein.

In some embodiments, an immune initiator is a therapeutic molecule. Non-limiting examples of such therapeutic molecules are described herein and include, but are not limited to, cytokines, chemokines, single chain antibodies (agonistic or antagonistic), ligands (agonistic or antagonistic), co-stimulatory receptors/ligands and the like. In another embodiment, an immune initiator is a STING agonist. In another embodiment, an immune initiator is arginine In another embodiment, an immune initiator is at least one enzyme of a catabolic pathway. Non-limiting examples of such catabolic pathways are described herein and include, but are not limited to, enzymes involved in the catabolism of a harmful metabolite. In another embodiment, an immune initiator is at least one molecule produced by at least one enzyme of a biosynthetic pathway. In another embodiment, an immune initiator is a metabolic converter. In other embodiments, the immune initiator may be a nucleic acid molecule that mediates RNA interference, microRNA response or inhibition, TLR response, antisense gene regulation, target protein binding (aptamer or decoy oligos), gene editing, such as CRISPR interference.

In specific embodiments, the one or more immune initiators, modulate, e.g., intensify, one or more of steps (1) lysis of tumor cells and/or uptake of tumor antigens, (2), activation of APCs and/or (3) priming and activation of T cells. In some embodiments, the one or more immune initiators modulate, e.g., intensify, one or more of steps (1) lysis of tumor cells and/or uptake of tumor antigens, (2) activation of APCs and/or (3) priming and activation of T cells. In some embodiments, the one or more immune initiators, modulate, e.g., intensify, one or more of steps (1) oncolysis and/or uptake of tumor antigens, (2) activation of APCs and/or (3) priming and activation of T cells. Any immune initiator may be combined with one or more additional same or different immune initiator(s), which modulate the same or a different step in the cancer immunity cycle.

In one embodiment, the one or more immune initiators which modulate oncolysis or tumor antigen uptake (step (1)). Non-limiting examples of immune initiators which modulate antigen acquisition are described herein and known in the art and include but are not limited to lytic peptides, CD47 blocking antibodies, SIRP-alpha and variants, TNFα, IFN-γ and SFU. In one embodiment, the one or more immune initiators which modulate activation of APCs (step (2)). Non-limiting examples of immune initiators modulate activation of APCs are described herein and known in the art and include but are not limited to Toll-like receptor agonists, STING agonists, CD40L, and GM-CSF. In one embodiment, the one or more immune initiators modulate, e.g., enhance, priming and activation of T cells (step (3)). Non-limiting examples of immune initiators which modulate, e.g., enhance, priming and activation of T cells are described herein and known in the art and include but are not limited to an anti-OX40 antibody, OXO40L, an anti-41BB antibody , 41BBL, an anti-GITR antibody, GITRL, anti-CD28 antibody, anti-CTLA4 antibody, anti-PD1 antibody, anti-PDL1 antibody, IL-15, and IL-12, etc.

As used herein the term “immune sustainer” or “sustainer” refers to a class of effectors or molecules, e.g., immune modulators, or substances. Immune sustainers may modulate, e.g., boost or enhance, one or more steps of the cancer immunity cycle, including (4) trafficking and infiltration; (5) recognition of cancer cells by T cells and T cell support; and/or (6) the ability to overcome immune suppression. In one embodiment, the immune sustainer may be administered in combination with a microorganism described herein. For example, a microorganism described herein is administered in combination with an immune sustainer but administered before, at the same time as, or after, the administration of the microorganism and/or the immune sustainer.

In some embodiments, the immune sustainer is a therapeutic molecule. Non-limiting examples of such therapeutic molecules are described herein and include cytokines, chemokines, single chain antibodies (agonistic or antagonistic), ligands (agonistic or antagonistic), and the like. In another embodiment, an immune sustainer is a therapeutic molecule produced by an enzyme. Non-limiting examples of such enzymes are described herein. In another embodiment, an immune sustainer is at least one enzyme of a biosynthetic pathway or a catabolic pathway. Non-limiting examples of such biosynthetic pathways are described herein and include, but are not limited to, enzymes involved in the production of arginine; and non-limiting examples of such catabolic pathways are described herein and include, but are not limited to, enzymes involved in the catalysis of kynurenine or enzymes involved in the catalysis of adenosine. In another embodiment, an immune sustainer is a metabolic converter. In other embodiments, the immune sustainer may be a nucleic acid molecule that mediates RNA interference, microRNA response or inhibition, TLR response, antisense gene regulation, target protein binding (aptamer or decoy oligos), gene editing, such as CRISPR interference.

In specific embodiments, the term “immune sustainer” may also refer to the reduction or elimination of a harmful molecule. In such instances, the term “immune sustainer” may also be used to refer to the one or more enzymes of the catabolic pathway which breaks down the harmful metabolite.

In some embodiments, the one or more immune sustainers modulate, e.g., boost, one or more of steps (4) T cell trafficking and infiltration, (5) recognition of cancer cells by T cells and/or T cell support and/or (6) the ability to overcome immune suppression. Any immune sustainer may be combined with one or more additional immune sustainer(s), which modulate the same or a different step. In some embodiments, the one or more immune sustainers, modulate, e.g., boost, one or more of steps (4) T cell trafficking and infiltration, (5) recognition of cancer cells by T cells and/or T cell support and/or (6) the ability to overcome immune suppression. In some embodiments, the one or more immune sustainers modulate, e.g., boost, one or more of steps (4) T cell trafficking and infiltration, (5) recognition of cancer cells by T cells and/or T cell support and/or (6) the ability to overcome immune suppression.

In one embodiment, the one or more immune sustainers modulate T cell trafficking and infiltration (step (4)). Non-limiting examples of immune sustainers which modulate T cell trafficking and infiltration are described herein and known in the art and include, but are not limited to, chemokines such as CXCL9 and CXCL10 or upstream activators which induce the expression of such cytokines. In one embodiment, the one or more immune sustainers modulate recognition of cancer cells by T cells and T cell support (step (5)). Non-limiting examples of immune sustainers which modulate recognition of cancer cells by T cells and T cell support are described herein and known in the art and include, but are not limited to, anti-PD1/PD-L1 antibodies (antagonistic), anti-CTLA-4 antibodies (antagonistic), kynurenine consumption, adenosine consumption, anti-OX40 antibodies (agonistic), anti-41BB antibodies (agonistic), and anti-GITR antibodies (agonistic). In one embodiment, the one or more immune sustainers modulate, e.g., enhance, the ability to overcome immune suppression (step (6)). Non-limiting examples of immune sustainers which modulate, e.g., enhance, the ability to overcome immune suppression are described herein and known in the art and include, but are not limited to, IL-15 and IL-12 and variants thereof.

Any one or more immune initiator(s) may be combined any one or more immune sustainer(s). Accordingly, in some embodiments, the one or more immune initiators modulate, e.g., intensify, one or more of steps (1) oncolysis, (2) activation of APCs and/or (3) priming and activation of T cells in combination with one or more immune sustainers, which modulate, e.g., boost, one or more of steps (4) T cell trafficking and infiltration, (5) recognition of cancer cells by T cells and/or T cell support and/or (6) the ability to overcome immune suppression.

In some embodiments, certain immune modulators act at multiple stages of the cancer immunity cycle, e.g., one or more stages of immune initiation, or one or more of immune sustenance, or at one or more stages of immune initiation and at one or more stages of immune sustenance.

As used herein a “metabolic conversion” refers to a chemical transformation which is the result of an enzyme-catalyzed reaction. The enzyme-catalyze reaction can be either biosynthetic or catabolic in nature.

As used herein, the term “metabolic converter” refers to one or more enzymes, which catalyze a chemical transformation, i.e., which consume, produce or convert a metabolite. In some embodiments, the term “metabolic converter” refers to the at least one molecule produced by the at least one enzyme of a biosynthetic pathway. A metabolic converter can consume a toxic or immunosuppressive metabolite or produce an anti-cancer metabolite, or both. Non-limiting examples of metabolic converters include kynurenine consumers, adenosine consumers, arginine producers and/or ammonia consumers, i.e., enzymes for the consumption of kynurenine or adenosine or for the production of arginine and/or consumption of ammonia. In one embodiment, a metabolic converter can be, for example, a human kynureninase enzyme (for example, EC 3.7.1.3). In another embodiment, a metabolic converter can be a nucleic acid, e.g., RNAi molecule (siRNA, miRNA, dsRNA), mRNA, antisense molecule, aptamer, or CRISPR/Cas 9 molecule, that increases or decreases endogenous expression of an enzyme(s) that catalyzes a chemical transformation, i.e., which consume, produce or convert a metabolite, in a tumor.

As used herein “wild-type” refers to an unmodified bacteria. For example, a wild-type bacteria has not been modified using genetic engineering. A wild-type bacteria, for example, has not been modified to express a non-native gene or to comprise an auxotrophy. In one embodiment, a wild-type bacteria is an E. coli Nissle bacteria.

A “bacteria chassis” or “chassis,” as used herein, refers to a bacteria that may comprise an auxotrophic modification, e.g., a mutation or deletion in dapA, thyA, or both, and/or deletion of a phage, and may stimulate an innate immune response; but the bacteria is not modified to comprise a non-native nucleic acid or gene or to express a non-native protein. In other words, a bacteria chassis refers to a bacteria that has not been modified to comprise a non-native immune modulator gene or to express a non-native immune modulator protein. In some embodiments, a chassis refers to a strain of Escherichia coli Nissle bacteria that may comprise an auxotrophic modification, e.g., a mutation or deletion in dapA, thyA, or both, and may stimulate an innate immune response, but is not modified to comprise a non-native gene or to express a non-native protein, e.g., has not been modified to comprise a non-native immune modulator nucleic acid or gene or to express a non-native immune modulator protein.

As used herein, “non-native” refers to a nucleic acid or a protein not normally present in a microorganism, e.g., an extra copy of an endogenous sequence, or a heterologous sequence such as a sequence from a different species, strain, or substrain of bacteria or virus, or a sequence that is modified and/or mutated as compared to the unmodified sequence from bacteria or virus of the same subtype. In some embodiments, the non-native nucleic acid sequence is a synthetic, non-naturally occurring sequence (see, e.g., Purcell et al., 2013). The non-native nucleic acid sequence may be a regulatory region, a promoter, a gene, and/or one or more genes in gene cassette. In some embodiments, “non-native” refers to two or more nucleic acid sequences that are not found in the same relationship to each other in nature. The non-native nucleic acid sequence may be present on a plasmid or chromosome.

As used herein, a “heterologous” gene or “heterologous sequence” refers to a nucleotide sequence that is not normally found in a given cell in nature. As used herein, a heterologous sequence encompasses a nucleic acid sequence that is exogenously introduced into a given cell. “Heterologous gene” includes a native gene, or fragment thereof, that has been introduced into the host cell in a form that is different from the corresponding native gene. For example, a heterologous gene may include a native coding sequence that is a portion of a chimeric gene to include a native coding sequence that is a portion of a chimeric gene to include non-native regulatory regions that is reintroduced into the host cell. A heterologous gene may also include a native gene, or fragment thereof, introduced into a non-native host cell. Thus, a heterologous gene may be foreign or native to the recipient cell; a nucleic acid sequence that is naturally found in a given cell but expresses an unnatural amount of the nucleic acid and/or the polypeptide which it encodes; and/or two or more nucleic acid sequences that are not found in the same relationship to each other in nature.

As used herein, the term “endogenous gene” refers to a native gene in its natural location in the genome of an organism. As used herein, the term “transgene” refers to a gene that has been introduced into the host organism, e.g., host bacterial cell, genome.

As used herein, the term “partial regression” refers to an inhibition of growth of a tumor, and/or the regression of a tumor, e.g., in size, after administration of the microorganism(s) and/or immune modulator(s) to a subject having the tumor. In one embodiment, a “partial regression” may refer to a regression of a tumor, e.g., in size, by at least about 5%, at least about 10%, at least about 15%, at least about 20%, at least about 25%, at least about 30%, at least about 40%, at least about 50%, at least about 60%, at least about 70%, at least about 80%, or at least about 90%. In another embodiment, a “partial regression” may refer to a decrease in the size of a tumor by at least about 5%, at least about 10%, at least about 15%, at least about 20%, at least about 25%, at least about 30%, at least about 40%, at least about 50%, at least about 60%, at least about 70%, at least about 75%, at least about 80%, or at least about 90%. In one embodiment, “partial regression” refers to the regression of a tumor, e.g., in size, but wherein the tumor is still detectable in the subject.

As used herein, the term “complete regression” refers to a complete regression of a tumor, e.g., in size, after administration of the microorganism(s) and/or immune modulator(s) to the subject having the tumor. When “complete regression” occurs the tumor is undetectable in the subject

As used herein, the term “percent response” refers to a percentage of subjects in a population of subjects who exhibit either a partial regression or a complete regression, as defined herein, after administration of a microorganism(s) and/or immune modulator(s). For example, in one embodiment, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, or about 95% of subjects in a population of subjects exhibit a partial response or a complete response.

As used herein, the term “stable disease” refers to a cancer or tumor that is neither growing nor shrinking “Stable disease” also refers to a disease state where no new tumors have developed, and a cancer or tumor has not spread to any new region or area of the body, e.g., by metastasis.

“Intratumoral administration” is meant to include any and all means for microorganism delivery to the intratumoral site and is not limited to intratumoral injection means. Examples of delivery means for the microorganisms is discussed in detail herein.

“Cancer” or “cancerous” is used to refer to a physiological condition that is characterized by unregulated cell growth. In some embodiments, cancer refers to a tumor. “Tumor” is used to refer to any neoplastic cell growth or proliferation or any pre-cancerous or cancerous cell or tissue. A tumor may be malignant or benign. Types of cancer include, but are not limited to, adrenal cancer, adrenocortical carcinoma, anal cancer, appendix cancer, bile duct cancer, bladder cancer, bone cancer (e.g., Ewing sarcoma tumors, osteosarcoma, malignant fibrous histiocytoma), brain cancer (e.g., astrocytomas, brain stem glioma, craniopharyngioma, ependymoma), bronchial tumors, central nervous system tumors, breast cancer, Castleman disease, cervical cancer, colon cancer, rectal cancer, colorectal cancer, endometrial cancer, esophageal cancer, eye cancer, gallbladder cancer, gastrointestinal cancer, gastrointestinal carcinoid tumors, gastrointestinal stromal tumors, gestational trophoblastic disease, heart cancer, Kaposi sarcoma, kidney cancer, laryngeal cancer, hypopharyngeal cancer, leukemia (e.g., acute lymphoblastic leukemia, acute myeloid leukemia, chronic lymphocytic leukemia, chronic myelogenous leukemia), liver cancer, lung cancer, lymphoma (e.g., AIDS-related lymphoma, Burkitt lymphoma, cutaneous T cell lymphoma, Hodgkin lymphoma, Non-Hodgkin lymphoma, primary central nervous system lymphoma), malignant mesothelioma, multiple myeloma, myelodysplastic syndrome, nasal cavity cancer, paranasal sinus cancer, nasopharyngeal cancer, neuroblastoma, oral cavity cancer, oropharyngeal cancer, osteosarcoma, ovarian cancer, pancreatic cancer, penile cancer, pituitary tumors, prostate cancer, retinoblastoma, rhabdomyosarcoma, rhabdoid tumor, salivary gland cancer, sarcoma, skin cancer (e.g., basal cell carcinoma, melanoma), small intestine cancer, stomach cancer, teratoid tumor, testicular cancer, throat cancer, thymus cancer, thyroid cancer, unusual childhood cancers, urethral cancer, uterine cancer, uterine sarcoma, vaginal cancer, vulvar cancer, Waldenström macroglobulinemia, and Wilms tumor. Side effects of cancer treatment may include, but are not limited to, opportunistic autoimmune disorder(s), systemic toxicity, anemia, loss of appetite, irritation of bladder lining, bleeding and bruising (thrombocytopenia), changes in taste or smell, constipation, diarrhea, dry mouth, dysphagia, edema, fatigue, hair loss (alopecia), infection, infertility, lymphedema, mouth sores, nausea, pain, peripheral neuropathy, tooth decay, urinary tract infections, and/or problems with memory and concentration (National Cancer Institute).

As used herein, “abscopal” and “abscopal effect” refers to an effect in which localized treatment of a tumor not only shrinks or otherwise affects the tumor being treated, but also shrinks or otherwise affects other tumors outside the scope of the localized treatment. In some embodiments, the bacteria may elicit an abscopal effect. In some embodiments, no abscopal effect is observed upon administration of the bacteria.

In any of these embodiments in which abscopal effect is observed, timing of tumor growth in a tumor of the same type which is distal to the administration site is delayed by at least about 0 to 2 days, at least about 2 to 4 days, at least about 4 to 6 days, at least about 6 to 8 days, at least about 8 to 10 days, at least about 10 to 12 days, at least about 12 to 14 days, at least about 14 to 16 days, at least about 16 to 18 days, at least about 18 to 20 days, at least about 20 to 25 days, at least about 25 to 30 days, at least about 30 to 35 days of the same type relative to the tumor growth (tumor volume) in a naive animal or subject.

In any of these embodiments in which an abscopal effect is observed, timing of tumor growth as measured in tumor volume in a distal tumor of the same type is delayed by at least about 0 to 2 weeks, at least about 2 to 4 weeks, at least about 4 to 6 weeks, at least about 6 to 8 weeks, at least about 8 to 10 weeks, at least about 10 to 12 weeks, at least about 12 to 14 weeks, at least about 14 to 16 weeks, at least about 16 to 18 weeks, at least about 18 to 20 weeks, at least about 20 to 25 weeks, at least about 25 to 30 weeks, at least about 30 to 35 weeks, at least about 35 to 40 weeks, at least about 40 to 45 weeks, at least about 45 to 50 weeks, at least about 50 to 55 weeks, at least about 55 to 60 weeks, at least about 60 to 65 weeks, at least about 65 to 70 weeks, at least about 70 to 80 weeks, at least about 80 to 90 weeks, or at least about 90 to 100 in a tumor re-challenge relative to the tumor growth (tumor volume) in a naive animal or subject.

In any of these embodiments in which abscopal effect is observed, timing of tumor growth as measured in tumor volume in a tumor distal to the administration site of the same type is delayed by at least about 0 to 2 years, at least about 2 to 4 years, at least about 4 to 6 years, at least about 6 to 8 years, at least about 8 to 10 years, at least about 10 to 12 years, at least about 12 to 14 years, at least about 14 to 16 years, at least about 16 to 18 years, at least about 18 to 20 years, at least about 20 to 25 years, at least about 25 to 30 years, at least about 30 to 35 years, at least about 35 to 40 years, at least about 40 to 45 years, at least about 45 to 50 years, at least about 50 to 55 years, at least about 55 to 60 years, at least about 60 to 65 years, at least about 65 to 70 years, at least about 70 to 80 years, at least about 80 to 90 years, or at least about 90 to 100 in a tumor re-challenge relative to the tumor growth (tumor volume) in a naive animal or subject.

In yet another embodiment, survival rate is at least about 1.0-1.2-fold, at least about 1.2-1A-fold, at least about 1.4-1.6-fold, at least about 1.6-1.8-fold, at least about 1.8-2-fold, or at least about two-fold greater in a tumor re-challenge as compared to the tumor growth (tumor volume) in a naive subject. In yet another embodiment, survival rate is at least about 2 to 3-fold, at least about 3 to 4-fold, at least about 4 to 5-fold, at least about 5 to 6-fold, at least about 6 to 7-fold, at least about 7 to 8-fold, at least about 8 to 9-fold, at least about 9 to 10-fold, at least about 10 to 15-fold, at least about 15 to 20-fold, at least about 20 to 30-fold, at least about 30 to 40-fold, or at least about 40 to 50-fold, at least about 50 to 100-fold, at least about 100 to 500-hundred-fold, or at least about 500 to 1000-fold greater in a tumor re-challenge as compared to the tumor growth (tumor volume) in a naive subject. In this example, “tumor re-challenge” may also include metastasis formation which may occur in a subject at a certain stage of cancer progression.

Immunological memory represents an important aspect of the immune response in mammals. Memory responses form the basis for the effectiveness of vaccines against cancer cells. As used herein, the term “immune memory” or “immunological memory” refers to a state in which long-lived antigen-specific lymphocytes are available and are capable of rapidly mounting responses upon repeat exposure to a particular antigen. The importance of immunological memory in cancer immunotherapy is known, and the trafficking properties and long-lasting anti-tumor capacity of memory T cells play a crucial role in the control of malignant tumors and prevention of metastasis or reoccurrence. Immunological memory exists for both B lymphocytes and for T cells, and is now believed to exist in a large variety of other immune cells, including NK cells, macrophages, and monocytes. (see e.g., Farber et al., Immunological memory: lessons from the past and a look to the future (Nat. Rev. Immunol. (2016) 16: 124-128). Memory B cells are plasma cells that are able to produce antibodies for a long time. The memory B cell has already undergone clonal expansion and differentiation and affinity maturation, so it is able to divide multiple times faster and produce antibodies with much higher affinity. Memory T cells can be both CD4+ and CD8+. These memory T cells do not require further antigen stimulation to proliferate therefore they do not need a signal via MHC.

Immunological memory can, for example, be measured in an animal model by re-challenging the animal model upon achievement of complete regression upon treatment with the microorganism. The animal is then implanted with cancer cells from the cancer cell line and growth is monitored and compared to an age matched naïve animal of the same type which had not previously been exposed to the tumor. Such a tumor re-challenge is used to demonstrate systemic and long term immunity against tumor cells and may represent the ability to fight off future recurrence or metastasis formation. Such an experiment is described herein using the A20 tumor model in the Examples. Immunological memory would prevent or slow the reoccurrence of the tumor in the re-challenged animal relative to the naïve animal. On a cellular level, formation of immunological memory can be measured by expansion and/or persistence of tumor antigen specific memory or effector memory T cells.

In some embodiments, immunological memory is achieved in a subject upon administration of the compositions described herein. In some embodiments, immunological memory is achieved cancer patient upon administration of the compositions described herein.

In some embodiments, a complete response is achieved in a subject upon administration of the compositions described herein. In some embodiments, a complete response is achieved in a cancer patient upon administration of the compositions described herein.

In some embodiments, a complete remission is achieved in a subject upon administration of the compositions described herein. In some embodiments, a complete remission is achieved in a cancer patient upon administration of the compositions described herein.

In some embodiments, a partial response is achieved in a subject upon administration of the compositions described herein. In some embodiments, a partial response is achieved in a cancer patient upon administration of the compositions described herein.

In some embodiments, stable disease is achieved in a subject upon administration of the compositions described herein. In some embodiments, a partial response is achieved in a cancer patient upon administration of the compositions described herein.

In some embodiments, a subset of subjects within a group achieves a partial or complete response upon administration of the compositions described herein. In some embodiments, a subset of patients within a group achieve a partial or complete response upon administration of the compositions described herein.

In any of these embodiments in which immunological memory is observed, timing of tumor growth is delayed by at least about 0 to 2 days, at least about 2 to 4 days, at least about 4 to 6 days, at least about 6 to 8 days, at least about 8 to 10 days, at least about 10 to 12 days, at least about 12 to 14 days, at least about 14 to 16 days, at least about 16 to 18 days, at least about 18 to 20 days, at least about 20 to 25 days, at least about 25 to 30 days, at least about 30 to 35 days in a tumor re-challenge relative to the tumor growth (tumor volume) in a naive animal or subject.

In any of these embodiments in which immunological memory is observed, timing of tumor growth as measured in tumor volume delayed by at least about 0 to 2 weeks, at least about 2 to 4 weeks, at least about 4 to 6 weeks, at least about 6 to 8 weeks, at least about 8 to 10 weeks, at least about 10 to 12 weeks, at least about 12 to 14 weeks, at least about 14 to 16 weeks, at least about 16 to 18 weeks, at least about 18 to 20 weeks, at least about 20 to 25 weeks, at least about 25 to 30 weeks, at least about 30 to 35 weeks, at least about 35 to 40 weeks, at least about 40 to 45 weeks, at least about 45 to 50 weeks, at least about 50 to 55 weeks, at least about 55 to 60 weeks, at least about 60 to 65 weeks, at least about 65 to 70 weeks, at least about 70 to 80 weeks, at least about 80 to 90 weeks, or at least about 90 to 100 in a tumor re-challenge relative to the tumor growth (tumor volume) in a naive animal or subject.

In any of these embodiments in which immunological memory is observed, timing of tumor growth as measured in tumor volume delayed by at least about 0 to 2 years, at least about 2 to 4 years, at least about 4 to 6 years, at least about 6 to 8 years, at least about 8 to 10 years, at least about 10 to 12 years, at least about 12 to 14 years, at least about 14 to 16 years, at least about 16 to 18 years, at least about 18 to 20 years, at least about 20 to 25 years, at least about 25 to 30 years, at least about 30 to 35 years, at least about 35 to 40 years, at least about 40 to 45 years, at least about 45 to 50 years, at least about 50 to 55 years, at least about 55 to 60 years, at least about 60 to 65 years, at least about 65 to 70 years, at least about 70 to 80 years, at least about 80 to 90 years, or at least about 90 to 100 in a tumor re-challenge relative to the tumor growth (tumor volume) in a naive animal or subject.

In yet another embodiment, survival rate is at least about 1.0-1.2-fold, at least about 1.2-1.4-fold, at least about 1.4-1.6-fold, at least about 1.6-1.8-fold, at least about 1.8-2-fold, or at least about two-fold greater in a tumor re-challenge as compared to the tumor growth (tumor volume) in a naive subject. In yet another embodiment, survival rate is at least about 2 to 3-fold, at least about 3 to 4-fold, at least about 4 to 5-fold, at least about 5 to 6-fold, at least about 6 to 7-fold, at least about 7 to 8-fold, at least about 8 to 9-fold, at least about 9 to 10-fold, at least about 10 to 15-fold, at least about 15 to 20-fold, at least about 20 to 30-fold, at least about 30 to 40-fold, or at least about 40 to 50-fold, at least about 50 to 100-fold, at least about 100 to 500-hundred-fold, or at least about 500 to 1000-fold greater in a tumor re-challenge as compared to the tumor growth (tumor volume) in a naive subject.

As used herein, “hot tumors” refer to tumors, which are T cell inflamed, i.e., associated with a high abundance of T cells infiltrating into the tumor. “Cold tumors” are characterized by the absence of effector T cells infiltrating the tumor and are further grouped into “immune excluded” tumors, in which immune cells are attracted to the tumor but cannot infiltrate the tumor microenvironment, and “immune ignored” phenotypes, in which no recruitment of immune cells occurs at all (further reviewed in Van der Woude et al., Migrating into the Tumor: a Roadmap for T Cells. Trends Cancer. 2017 November; 3(11):797-808).

“Hypoxia” is used to refer to reduced oxygen supply to a tissue as compared to physiological levels, thereby creating an oxygen-deficient environment. “Normoxia” refers to a physiological level of oxygen supply to a tissue. Hypoxia is a hallmark of solid tumors and characterized by regions of low oxygen and necrosis due to insufficient perfusion (Groot et al., 2007).

As used herein, the term “low oxygen” is meant to refer to a level, amount, or concentration of oxygen (O₂) that is lower than the level, amount, or concentration of oxygen that is present in the atmosphere (e.g., <21% O_{2;} <160 torr O₂)). Thus, the term “low oxygen condition or conditions” or “low oxygen environment” refers to conditions or environments containing lower levels of oxygen than are present in the atmosphere.

In some embodiments, the term “low oxygen” is meant to refer to the level, amount, or concentration of oxygen (O₂) found in a mammalian gut, e.g., lumen, stomach, small intestine, duodenum, jejunum, ileum, large intestine, cecum, colon, distal sigmoid colon, rectum, and anal canal. In some embodiments, the term “low oxygen” is meant to refer to a level, amount, or concentration of O₂ that is 0-60 mmHg O₂ (0-60 torr O₂) (e.g., 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45,46, 47, 48, 49, 50, 51, 52, 53, 54, 55, 56, 57, 58, 59, and 60 mmHg O₂), including any and all incremental fraction(s) thereof (e.g., 0.2 mmHg, 0.5 mmHg O₂, 0.75 mmHg O₂, 1.25 mmHg O₂, 2.175 mmHg O₂, 3.45 mmHg O₂, 3.75 mmHg O₂, 4.5 mmHg O₂, 6.8 mmHg O₂, 11.35 mmHg 02, 46.3 mmHg O₂, 58.75 mmHg, etc., which exemplary fractions are listed here for illustrative purposes and not meant to be limiting in any way). In some embodiments, “low oxygen” refers to about 60 mmHg O₂ or less (e.g., 0 to about 60 mmHg O₂). The term “low oxygen” may also refer to a range of O₂ levels, amounts, or concentrations between 0-60 mmHg O₂(inclusive), e.g., 0-5 mmHg O₂, <1.5 mmHg O₂, 6-10 mmHg, <8 mmHg, 47-60 mmHg, etc. which listed exemplary ranges are listed here for illustrative purposes and not meant to be limiting in any way. See, for example, Albenberg et al., Gastroenterology, 147(5): 1055-1063 (2014); Bergofsky et al., J Clin. Invest., 41(11): 1971-1980 (1962); Crompton et al., J Exp. Biol., 43: 473-478 (1965); He et al., PNAS (USA), 96: 4586-4591 (1999); McKeown, Br. J. Radiol., 87:20130676 (2014) (doi: 10.1259/brj.20130676), each of which discusses the oxygen levels found in the mammalian gut of various species and each of which are incorporated by reference herewith in their entireties.

In some embodiments, the term “low oxygen” is meant to refer to the level, amount, or concentration of oxygen (O₂) found in a mammalian organ or tissue other than the gut, e.g., urogenital tract, tumor tissue, etc. in which oxygen is present at a reduced level, e.g., at a hypoxic or anoxic level. In some embodiments, “low oxygen” is meant to refer to the level, amount, or concentration of oxygen (O₂) present in partially aerobic, semi aerobic, microaerobic, nonaerobic, microoxic, hypoxic, anoxic, and/or anaerobic conditions. For example, Table 1 summarizes the amount of oxygen present in various organs and tissues. In some embodiments, the level, amount, or concentration of oxygen (O₂) is expressed as the amount of dissolved oxygen (“DO”) which refers to the level of free, non-compound oxygen (O₂) present in liquids and is typically reported in milligrams per liter (mg/L), parts per million (ppm; 1 mg/L=1 ppm), or in micromoles (umole) (1 umole O₂=0.022391 mg/L O₂). Fondriest Environmental, Inc., “Dissolved Oxygen”, Fundamentals of Environmental Measurements, 19 Nov 2013, www.fondriest.com/environmental-measurements/parameters/water-quality/dissolved-oxygen/>.

In some embodiments, the term “low oxygen” is meant to refer to a level, amount, or concentration of oxygen (O₂) that is about 6.0 mg/L DO or less, e.g., 6.0 mg/L, 5.0 mg/L, 4.0 mg/L, 3.0 mg/L, 2.0 mg/L, 1.0 mg/L, or 0 mg/L, and any fraction therein, e.g., 3.25 mg/L, 2.5 mg/L, 1.75 mg/L, 1.5 mg/L, 1.25 mg/L, 0.9 mg/L, 0.8 mg/L, 0.7 mg/L, 0.6 mg/L, 0.5 mg/L, 0.4 mg/L, 0.3 mg/L, 0.2 mg/L and 0.1 mg/L DO, which exemplary fractions are listed here for illustrative purposes and not meant to be limiting in any way. The level of oxygen in a liquid or solution may also be reported as a percentage of air saturation or as a percentage of oxygen saturation (the ratio of the concentration of dissolved oxygen (O₂) in the solution to the maximum amount of oxygen that will dissolve in the solution at a certain temperature, pressure, and salinity under stable equilibrium). Well-aerated solutions (e.g., solutions subjected to mixing and/or stirring) without oxygen producers or consumers are 100% air saturated.

In some embodiments, the term “low oxygen” is meant to refer to 40% air saturation or less, e.g., 40%, 39%, 38%, 37%, 36%, 35%, 34%, 33%, 32%, 31%, 30%, 29%, 28%, 27%, 26%, 25%, 24%, 23%, 22%, 21%, 20%, 19%, 18%, 17%, 16%, 15%, 14%, 13%, 12%, 11%, 10%, 9%, 8%, 7%, 6%, 5%, 4%, 3%, 2%, 1%, and 0% air saturation, including any and all incremental fraction(s) thereof (e.g., 30.25%, 22.70%, 15.5%, 7.7%, 5.0%, 2.8%, 2.0%, 1.65%, 1.0%, 0.9%, 0.8%, 0.75%, 0.68%, 0.5%. 0.44%, 0.3%, 0.25%, 0.2%, 0.1%, 0.08%, 0.075%, 0.058%, 0.04%. 0.032%, 0.025%, 0.01%, etc.) and any range of air saturation levels between 0-40%, inclusive (e.g., 0-5%, 0.05-0.1%, 0.1-0.2%, 0.1-0.5%, 0.5-2.0%, 0-10%, 5-10%, 10-15%, 15-20%, 20-25%, 25-30%, etc.).

The exemplary fractions and ranges listed here are for illustrative purposes and not meant to be limiting in any way. In some embodiments, the term “low oxygen” is meant to refer to 9% O₂ saturation or less, e.g., 9%, 8%, 7%, 6%, 5%, 4%, 3%, 2%, 1%, 0%, O₂ saturation, including any and all incremental fraction(s) thereof (e.g., 6.5%, 5.0%, 2.2%, 1.7%, 1.4%, 0.9%, 0.8%, 0.75%, 0.68%, 0.5%. 0.44%, 0.3%, 0.25%, 0.2%, 0.1%, 0.08%, 0.075%, 0.058%, 0.04%. 0.032%, 0.025%, 0.01%, etc.) and any range of O₂ saturation levels between 0-9%, inclusive (e.g., 0-5%, 0.05-0.1%, 0.1-0.2%, 0.1-0.5%, 0.5-2.0%, 0-8%, 5-7%, 0.3-4.2% O₂, etc.). The exemplary fractions and ranges listed here are for illustrative purposes and not meant to be limiting in any way.

TABLE 1

| Compartment | Oxygen Tension |
|---|---|
| stomach | ~60 torr (e.g., 58 +/− 15 torr) |
| duodenum and first part of jejunum | ~30 torr (e.g., 32 +/− 8 torr); ~20% |
| | oxygen in ambient air |
| Ileum (mid- small intestine) | ~10 torr; ~6% oxygen in ambient air |
| | (e.g., 11 +/− 3 torr) |
| Distal sigmoid colon | ~3 torr (e.g., 3 +/− 1 torr) |
| colon | <2 torr |
| Lumen of cecum | <1 torr |
| tumor | <32 torr (most tumors are <15 torr) |

As used herein, the term “gene” or “gene sequence” refers to any sequence expressing a polypeptide or protein, including genomic sequences, cDNA sequences, naturally occurring sequences, artificial sequences, and codon optimized sequences. The term “gene” or “gene sequence” inter alia includes modification of endogenous genes, such as deletions, mutations, and expression of native and non-native genes under the control of a promoter that that they are not normally associated with in nature.

As used herein the terms “gene cassette” and “circuit” or “circuitry” inter alia refers to any sequence expressing a polypeptide or protein, including genomic sequences, cDNA sequences, naturally occurring sequences, artificial sequences, and codon optimized sequences includes modification of endogenous genes, such as deletions, mutations, and expression of native and non-native genes under the control of a promoter that that they are not normally associated with in nature.

An antibody generally refers to a polypeptide of the immunoglobulin family or a polypeptide comprising fragments of an immunoglobulin that is capable of noncovalently, reversibly, and in a specific manner binding a corresponding antigen. An exemplary antibody structural unit comprises a tetramer composed of two identical pairs of polypeptide chains, each pair having one “light” (about 25 kD) and one “heavy” chain (about 50-70 ID), connected through a disulfide bond.

As used herein, the term “antibody” or “antibodies” is meant to encompasses all variations of antibody and fragments thereof that possess one or more particular binding specificities. Thus, the term “antibody” or “antibodies” is meant to include full length antibodies, chimeric antibodies, humanized antibodies, single chain antibodies (ScFv, camelids), Fab, Fab′, multimeric versions of these fragments (e.g., F(ab′)2), single domain antibodies (sdAB, V_{H}H fragments), heavy chain antibodies (HCAb), nanobodies, diabodies, and minibodies. Antibodies can have more than one binding specificity, e.g. be bispecific. The term “antibody” is also meant to include so-called antibody mimetics, i.e., which can specifically bind antigens but do not have an antibody-related structure.

A “single-chain antibody” or “single-chain antibodies” typically refers to a peptide comprising a heavy chain of an immunoglobulin, a light chain of an immunoglobulin, and optionally a linker or bond, such as a disulfide bond. The single-chain antibody lacks the constant Fc region found in traditional antibodies. In some embodiments, the single-chain antibody is a naturally occurring single-chain antibody, e.g., a camelid antibody. In some embodiments, the single-chain antibody is a synthetic, engineered, or modified single-chain antibody. In some embodiments, the single-chain antibody is capable of retaining substantially the same antigen specificity as compared to the original immunoglobulin despite the addition of a linker and the removal of the constant regions. In some aspects, the single chain antibody can be a “scFv antibody”, which refers to a fusion protein of the variable regions of the heavy (VH) and light chains (VL) of immunoglobulins (without any constant regions), optionally connected with a short linker peptide of ten to about 25 amino acids, as described, for example, in U.S. Pat. No. 4,946,778, the contents of which is herein incorporated by reference in its entirety. The Fv fragment is the smallest fragment that holds a binding site of an antibody, which binding site may, in some aspects, maintain the specificity of the original antibody. Techniques for the production of single chain antibodies are described in U.S. Pat. No. 4,946,778.

As used herein, the term “polypeptide” includes “polypeptide” as well as “polypeptides,” and refers to a molecule composed of amino acid monomers linearly linked by amide bonds (i.e., peptide bonds). The term “polypeptide” refers to any chain or chains of two or more amino acids, and does not refer to a specific length of the product. Thus, “peptides,” “dipeptides,” “tripeptides, “oligopeptides,” “protein,” “amino acid chain,” or any other term used to refer to a chain or chains of two or more amino acids, are included within the definition of “polypeptide,” and the term “polypeptide” may be used instead of, or interchangeably with any of these terms. The term “polypeptide” is also intended to refer to the products of post-expression modifications of the polypeptide, including but not limited to glycosylation, acetylation, phosphorylation, amidation, derivatization, proteolytic cleavage, or modification by non-naturally occurring amino acids. A polypeptide of the invention may be of a size of about 3 or more, 5 or more, 10 or more, 20 or more, 25 or more, 50 or more, 75 or more, 100 or more, 200 or more, 500 or more, 1,000 or more, or 2,000 or more amino acids.

An “isolated” polypeptide or a fragment, variant, or derivative thereof refers to a polypeptide that is not in its natural milieu. No particular level of purification is required. Recombinantly produced polypeptides and proteins expressed in host cells, including but not limited to bacterial or mammalian cells, are considered isolated for purposed of the invention, as are native or recombinant polypeptides which have been separated, fractionated, or partially or substantially purified by any suitable technique. Recombinant peptides, polypeptides or proteins refer to peptides, polypeptides or proteins produced by recombinant DNA techniques, i.e. produced from cells, microbial or mammalian, transformed by an exogenous recombinant DNA expression construct encoding the polypeptide. Proteins or peptides expressed in most bacterial cultures will typically be free of glycan. Fragments, derivatives, analogs or variants of the foregoing polypeptides, and any combination thereof are also included as polypeptides. The terms “fragment,” “variant,” “derivative” and “analog” include polypeptides having an amino acid sequence sufficiently similar to the amino acid sequence of the original peptide and include any polypeptides, which retain at least one or more properties of the corresponding original polypeptide. Fragments of polypeptides of the present invention include proteolytic fragments, as well as deletion fragments. Fragments also include specific antibody or bioactive fragments or immunologically active fragments derived from any polypeptides described herein. Variants may occur naturally or be non-naturally occurring. Non-naturally occurring variants may be produced using mutagenesis methods known in the art. Variant polypeptides may comprise conservative or non-conservative amino acid substitutions, deletions or additions.

Polypeptides also include fusion proteins. As used herein, the term “variant” includes a fusion protein, which comprises a sequence of the original peptide or sufficiently similar to the original peptide. As used herein, the term “fusion protein” refers to a chimeric protein comprising amino acid sequences of two or more different proteins. Typically, fusion proteins result from well known in vitro recombination techniques. Fusion proteins may have a similar structural function (but not necessarily to the same extent), and/or similar regulatory function (but not necessarily to the same extent), and/or similar biochemical function (but not necessarily to the same extent) and/or immunological activity (but not necessarily to the same extent) as the individual original proteins which are the components of the fusion proteins. “Derivatives” include but are not limited to peptides, which contain one or more naturally occurring amino acid derivatives of the twenty standard amino acids. “Similarity” between two peptides is determined by comparing the amino acid sequence of one peptide to the sequence of a second peptide. An amino acid of one peptide is similar to the corresponding amino acid of a second peptide if it is identical or a conservative amino acid substitution. Conservative substitutions include those described in Dayhoff, M. O., ed., The Atlas of Protein Sequence and Structure 5, National Biomedical Research Foundation, Washington, D.C. (1978), and in Argos, EMBO J. 8 (1989), 779-785. For example, amino acids belonging to one of the following groups represent conservative changes or substitutions: Ala, Pro, Gly, Gln, Asn, Ser, Thr, Cys, Ser, Tyr, Thr, Val, Ile, Leu, Met, Ala, Phe, Lys, Arg, His, Phe, Tyr, Trp, His, Asp, and Glu.

In any of these combination embodiments, an immune modulator may be fused to a stabilizing polypeptide. Such stabilizing polypeptides are known in the art and include Fc proteins. In some embodiments, the fusion proteins are Fc fusion proteins, such as IgG Fc fusion proteins or IgA Fc fusion proteins.

In some embodiments, an immune modulator, is covalently fused to the stabilizing polypeptide through a peptide linker or a peptide bond. In some embodiments, the stabilizing polypeptide comprises an immunoglobulin Fc polypeptide. In some embodiments, the immunoglobulin Fc polypeptide comprises at least a portion of an immunoglobulin heavy chain CH2 constant region. In some embodiments, the immunoglobulin Fc polypeptide comprises at least a portion of an immunoglobulin heavy chain CH3 constant region. In some embodiments, the immunoglobulin Fc polypeptide comprises at least a portion of an immunoglobulin heavy chain CH1 constant region. In some embodiments, the immunoglobulin Fc polypeptide comprises at least a portion of an immunoglobulin variable hinge region. In some embodiments, the immunoglobulin Fc polypeptide comprises at least a portion of an immunoglobulin variable hinge region, immunoglobulin heavy chain CH2 constant region and an immunoglobulin heavy chain CH3 constant region. In some embodiments, the immunoglobulin Fc polypeptide is a human IgG4 Fc polypeptide. In some embodiments, the linker comprises a glycine rich peptide. In some embodiments, the glycine rich peptide comprises the sequence [GlyGlyGlyGlySer]n where n is 1,2,3,4,5 or 6 (SEQ ID NO: 1245). In some embodiments, the fusion protein comprises a SIRPα IgG FC fusion polypeptide. In some embodiments, the fusion protein comprises a SIRPα IgG4 Fc polypeptide. In some embodiments, the glycine rich peptide linker comprises the sequence SGGGGSGGGGSGGGGS (SEQ ID NO: 1121). In some embodiments, the N terminus of SIRPα is covalently fused to the C terminus of a IgG4 Fc through the peptide linker comprising SGGGGSGGGGSGGGGS (SEQ ID NO: 1121).

In some embodiments, the immune modulator is a multimeric polypeptide. In some embodiments, the polypeptide is a dimer. Non-limiting example of a dimeric proteins include cytokines, such as IL-15 (heterodimer). In some embodiments, the immune modulator comprises one or more polypeptides wherein the one or more polypeptides comprise a first monomer and a second monomer. In some embodiments, the first monomer polypeptide is covalently linked to a second monomer polypeptide through a peptide linker or a peptide bond. In some embodiments, the linker comprises a glycine rich peptide. In some embodiments, the first and the second monomer have the same polypeptide sequence. In some embodiments, the first and the second monomer have each have a different polypeptide sequence. In some embodiments, the first monomer is a IL-12 p35 polypeptide and the second monomer is a IL-12 p40 polypeptide. In some embodiments, the linker comprises GGGGSGGGS (SEQ ID NO: 1244).

In some embodiments, the immune modulator is a hIGg4 fusion protein which comprises a hIgG4 portion that has about 80%, 81%, 82%, 83%, 84%, 85%, 86%, 87%, 88%, 89%, 90%, 91%, 92%, 93%, 94%, 95%, 96%, 97%, 98%, or 99% identity with one or more of SEQ ID NO: 1117. In another embodiment, the hIgG4 portion comprises SEQ ID NO: 1117. In yet another embodiment, the hIgG4 portion of the polypeptide consists of SEQ ID NO: 1117.

In some embodiments, the fusion protein comprises a linker portion that has about 80%, 81%, 82%, 83%, 84%, 85%, 86%, 87%, 88%, 89%, 90%, 91%, 92%, 93%, 94%, 95%, 96%, 97%, 98%, or 99% identity with one or more of SEQ ID NO: 1121. In another embodiment, the linker portion comprises SEQ ID NO: 1121. In yet another embodiment, the linker portion of the polypeptide consists of SEQ ID NO: 1121.

In some embodiments, effector function of an immune modulator can be improved through fusion to another polypeptide that facilitates effector function. A non-limiting example of such a fusion is the fusion of IL-15 to the Sushi domain of IL-15Ralpha, as described herein. In some embodiments, accordingly, a first monomer polypeptide is a IL-15 monomer and the second monomer is a IL-15R alpha sushi domain polypeptide.

As used herein, the term “sufficiently similar” means a first amino acid sequence that contains a sufficient or minimum number of identical or equivalent amino acid residues relative to a second amino acid sequence such that the first and second amino acid sequences have a common structural domain and/or common functional activity. For example, amino acid sequences that comprise a common structural domain that is at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or at least about 100%, identical are defined herein as sufficiently similar. Preferably, variants will be sufficiently similar to the amino acid sequence of the peptides of the invention. Such variants generally retain the functional activity of the peptides of the present invention. Variants include peptides that differ in amino acid sequence from the native and wild-type peptide, respectively, by way of one or more amino acid deletion(s), addition(s), and/or substitution(s). These may be naturally occurring variants as well as artificially designed ones.

As used herein the term “linker”, “linker peptide” or “peptide linkers” or “linker” refers to synthetic or non-native or non-naturally-occurring amino acid sequences that connect or link two polypeptide sequences, e.g., that link two polypeptide domains. As used herein the term “synthetic” refers to amino acid sequences that are not naturally occurring. Exemplary linkers are described herein. Additional exemplary linkers are provided in US 20140079701, the contents of which are herein incorporated by reference in its entirety. In some embodiments, the linker is a glycine rich linker. In some embodiments, the linker is (Gly-Gly-Gly-Gly-Ser)n. In some embodiments, the linker comprises SEQ ID NO: 979.

The immune system is typically most broadly divided into two categories-innate immunity and adaptive immunity—although the immune responses associated with these immunities are not mutually exclusive. “Innate immunity” refers to non-specific defense mechanisms that are activated immediately or within hours of a foreign agent's or antigen's appearance in the body. These mechanisms include physical barriers such as skin, chemicals in the blood, and immune system cells, such as dendritic cells (DCs), leukocytes, phagocytes, macrophages, neutrophils, and natural killer cells (NKs), that attack foreign agents or cells in the body and alter the rest of the immune system to the presence of the foreign agents. During an innate immune response, cytokines and chemokines are produced which in combination with the presentation of immunological antigens, work to activate adaptive immune cells and initiate a full blown immunologic response. “Adaptive immunity” or “acquired immunity” refers to antigen-specific immune response. The antigen must first be processed or presented by antigen presenting cells (APCs). An antigen-presenting cell or accessory cell is a cell that displays antigens directly or complexed with major histocompatibility complexes (MHCs) on their surfaces. Professional antigen-presenting cells, including macrophages, B cells, and dendritic cells, specialize in presenting foreign antigen to T helper cells in a MHC-II restricted manner, while other cell types can present antigen originating inside the cell to cytotoxic T cells in a MHC-I restricted manner. Once an antigen has been presented and recognized, the adaptive immune system activates an army of immune cells specifically designed to attack that antigen. Like the innate system, the adaptive system includes both humoral immunity components (B lymphocyte cells) and cell-mediated immunity (T lymphocyte cells) components. B cells are activated to secrete antibodies, which travel through the bloodstream and bind to the foreign antigen. Helper T cells (regulatory T cells, CD4+ cells) and cytotoxic T cells (CTL, CD8+ cells) are activated when their T cell receptor interacts with an antigen-bound MHC molecule. Cytokines and co-stimulatory molecules help the T cells mature, which mature cells, in turn, produce cytokines which allows the production of priming and expansion of additional T cells sustaining the response. Once activated, the helper T cells release cytokines which regulate and direct the activity of different immune cell types, including APCs, macrophages, neutrophils, and other lymphocytes, to kill and remove targeted cells. Helper T cells also secrete extra signals that assist in the activation of cytotoxic T cells which also help to sustain the immune response. Upon activation, CTL undergoes clonal selection, in which it gains functions, divides rapidly to produce an army of activated effector cells, and forms long-lived memory T cells ready to rapidly respond to future threats. Activated CTL then travels throughout the body searching for cells that bear that unique MHC Class I and antigen. The effector CTLs release cytotoxins that form pores in the target cell's plasma membrane, causing apoptosis. Adaptive immunity also includes a “memory” that makes future responses against a specific antigen more efficient. Upon resolution of the infection, T helper cells and cytotoxic T cells die and are cleared away by phagocytes, however, a few of these cells remain as memory cells. If the same antigen is encountered at a later time, these memory cells quickly differentiate into effector cells, shortening the time required to mount an effective response.

An “immune checkpoint inhibitor” or “immune checkpoint” refers to a molecule that completely or partially reduces, inhibits, interferes with, or modulates one or more immune checkpoint proteins. Immune checkpoint proteins regulate T-cell activation or function, and are known in the art. Non-limiting examples include CTLA-4 and its ligands CD 80 and CD86, and PD-1 and its ligands PD-L1 and PD-L2. Immune checkpoint proteins are responsible for co-stimulatory or inhibitory interactions of T-cell responses, and regulate and maintain self-tolerance and physiological immune responses.

A “co-stimulatory” molecule or “co-stimulator” is an immune modulator that increases or activates a signal that stimulates an immune response or inflammatory response.

As used herein, an immune modulator that “inhibits” cancerous cells refers to a molecule that is capable of reducing cell proliferation, reducing tumor growth, and/or reducing tumor volume by at least about 10% to 20%, 20% to 25%, 25% to 30%, 30% to 40%, 40% to 50%, 50% to 60%, 60% to 70%, 70% to 75%, 75% to 80%, 80% to 85%, 85% to 90%, 90% to 95%, 95% to 99%, or more as compared to control, e.g., an untreated control.

As used herein, an immune modulator that “activates” or “stimulates” a biological molecule, e.g., cytokine, chemokine, immune modulatory metabolite, or any other immune modulatory agent, factor, or molecule, refers to an immune modulator that is capable of activating, increasing, enhancing, or promoting the biological activity, biological function, and/or number of that biological molecule, as compared to control, e.g., an untreated control under the same conditions.

“Bacteria for intratumoral administration” refer to bacteria that are capable of directing themselves to cancerous cells. Bacteria for intratumoral administration may be naturally capable of directing themselves to cancerous cells, necrotic tissues, and/or hypoxic tissues.

In some embodiments, bacteria that are not naturally capable of directing themselves to cancerous cells, necrotic tissues, and/or hypoxic tissues are genetically engineered to direct themselves to cancerous cells, necrotic tissues, and/or hypoxic tissues. Bacteria for intratumoral administration may be further engineered to enhance or improve desired biological properties, mitigate systemic toxicity, and/or ensure clinical safety. These species, strains, and/or subtypes may be attenuated, e.g., deleted for a toxin gene.

In some embodiments, bacteria for intratumoral administration have low infection capabilities. In some embodiments, bacteria for intratumoral administration are motile. In some embodiments, the bacteria for intratumoral administration are capable of penetrating deeply into the tumor, where standard treatments do not reach. In some embodiments, bacteria for intratumoral administration are capable of colonizing at least 20%, at least 30%, at least 40%, at least 50%, at least 60%, at least 70%, at least 80%, at least 90%, or at least 95% of a malignant tumor. Examples of bacteria for intratumoral administration include, but are not limited to, Bifidobacterium, Caulobacter, Clostridium, Escherichia coli, Listeria, Mycobacterium, Salmonella, Streptococcus, and Vibrio, e.g., Bifidobacterium adolescentis, Bifidobacterium bifidum, Bifidobacterium breve UCC2003, Bifidobacterium infantis, Bifidobacterium longum, Clostridium acetobutylicum, Clostridium butyricum, Clostridium butyricum M-55, Clostridium butyricum miyairi, Clostridium cochlearum, Clostridium felsineum, Clostridium histolyticum, Clostridium multifermentans, Clostridium novyi-NT, Clostridium paraputrificum, Clostridium pasteureanum, Clostridium pectinovorum, Clostridium perfringens, Clostridium roseum, Clostridium sporogenes, Clostridium tertium, Clostridium tetani, Clostridium tyrobutyricum, Corynebacterium parvum, Escherichia coli MG1655, Escherichia coli Nissle 1917, Listeria monocytogenes, Mycobacterium bovis, Salmonella choleraesuis, Salmonella typhimurium, and Vibrio cholera (Cronin et al., 2012; Forbes, 2006; Jain and Forbes, 2001; Liu et al., 2014; Morrissey et al., 2010; Nuno et al., 2013; Patyar et al., 2010; Cronin, et al., Mol Ther 2010; 18:1397-407). In some embodiments, the bacteria for intratumoral administration are non-pathogenic bacteria. In some embodiments, intratumoral administration is done via injection.

“Microorganism” refers to an organism or microbe of microscopic, submicroscopic, or ultramicroscopic size that typically consists of a single cell. Examples of microorganisms include bacteria, viruses, parasites, fungi, certain algae, protozoa, and yeast. In some aspects, the microorganism is modified (“modified microorganism”) from its native state. In certain embodiments, the modified microorganism is a modified bacterium. In some embodiments, the modified microorganism is a genetically engineered bacterium. In certain embodiments, the modified microorganism is a modified yeast. In other embodiments, the modified microorganism is a genetically engineered yeast.

As used herein, the term “recombinant microorganism” refers to a microorganism, e.g., bacterial, yeast, or viral cell, or bacteria, yeast, or virus, that has been genetically modified from its native state. Thus, a “recombinant bacterial cell” or “recombinant bacteria” refers to a bacterial cell or bacteria that have been genetically modified from their native state. For instance, a recombinant bacterial cell may have nucleotide insertions, nucleotide deletions, nucleotide rearrangements, and nucleotide modifications introduced into their DNA. These genetic modifications may be present in the chromosome of the bacteria or bacterial cell, or on a plasmid in the bacteria or bacterial cell. Recombinant bacterial cells disclosed herein may comprise exogenous nucleotide sequences on plasmids. Alternatively, recombinant bacterial cells may comprise exogenous nucleotide sequences stably incorporated into their chromosome.

A “programmed or engineered microorganism” refers to a microorganism, e.g., bacterial, yeast, or viral cell, or bacteria, yeast, or virus, that has been genetically modified from its native state to perform a specific function. Thus, a “programmed or bacterial cell” or “programmed or bacteria” refers to a bacterial cell or bacteria that has been genetically modified from its native state to perform a specific function. In certain embodiments, the programmed or bacterial cell has been modified to express one or more proteins, for example, one or more proteins that have a therapeutic activity or serve a therapeutic purpose. The programmed or bacterial cell may additionally have the ability to stop growing or to destroy itself once the protein(s) of interest have been expressed.

“Non-pathogenic bacteria” refer to bacteria that are not capable of causing disease or harmful responses in a host. In some embodiments, non-pathogenic bacteria are Gram-negative bacteria. In some embodiments, non-pathogenic bacteria are Gram-positive bacteria. In some embodiments, non-pathogenic bacteria do not contain lipopolysaccharides (LPS). In some embodiments, non-pathogenic bacteria are commensal bacteria. Examples of non-pathogenic bacteria include, but are not limited to certain strains belonging to the genus Bacillus, Bacteroides, Bifidobacterium, Brevibacteria, Clostridium, Enterococcus, Escherichia coli, Lactobacillus, Lactococcus, Saccharomyces, and Staphylococcus, e.g., Bacillus coagulans, Bacillus subtilis, Bacteroides fragilis, Bacteroides subtilis, Bacteroides thetaiotaomicron, Bifidobacterium bifidum, Bifidobacterium infantis, Bifidobacterium lactis, Bifidobacterium longum, Clostridium butyricum, Enterococcus faecium, Escherichia coli Nissle, Lactobacillus acidophilus, Lactobacillus bulgaricus, Lactobacillus casei, Lactobacillus johnsonii, Lactobacillus paracasei, Lactobacillus plantarum, Lactobacillus reuteri, Lactobacillus rhamnosus, Lactococcus lactis, and Saccharomyces boulardii (Sonnenborn et al., 2009; Dinleyici et al., 2014; U.S. Pat. No. 6,835,376; U.S. Pat. No. 6,203,797; U.S. Pat. No. 5,589,168; U.S. Pat. No. 7,731,976).

“Probiotic” is used to refer to live, non-pathogenic microorganisms, e.g., bacteria, which can confer health benefits to a host organism that contains an appropriate amount of the microorganism. In some embodiments, the host organism is a mammal. In some embodiments, the host organism is a human. In some embodiments, the probiotic bacteria are Gram-negative bacteria. In some embodiments, the probiotic bacteria are Gram-positive bacteria. Some species, strains, and/or subtypes of non-pathogenic bacteria are currently recognized as probiotic bacteria. Examples of probiotic bacteria include, but are not limited to certain strains belonging to the genus Bifidobacteria, Escherichia coli, Lactobacillus, and Saccharomyces, e.g., Bifidobacterium bifidum, Enterococcus faecium, Escherichia coli strain Nissle, Lactobacillus acidophilus, Lactobacillus bulgaricus, Lactobacillus paracasei, Lactobacillus plantarum, and Saccharomyces boulardii (Dinleyici et al., 2014; U.S. Pat. No. 5,589,168; U.S. Pat. No. 6,203,797; U.S. Pat. No. 6,835,376). The probiotic may be a variant or a mutant strain of bacterium (Arthur et al., 2012; Cuevas-Ramos et al., 2010; Olier et al., 2012; Nougayrede et al., 2006).

“Operably linked” refers a nucleic acid sequence that is joined to a regulatory region sequence in a manner which allows expression of the nucleic acid sequence, e.g., acts in cis. A regulatory region is a nucleic acid that can direct transcription of a gene of interest and may comprise promoter sequences, enhancer sequences, response elements, protein recognition sites, inducible elements, promoter control elements, protein binding sequences, 5′ and 3′ untranslated regions, transcriptional start sites, termination sequences, polyadenylation sequences, and introns.

An “inducible promoter” refers to a regulatory region that is operably linked to one or more genes, wherein expression of the gene(s) is increased in the presence of an inducer of said regulatory region. In one embodiment, an inducible promoter is a salicylate promoter. In another embodiment, an inducible promoter is a cumate promoter. In another embodiment, an inducible promoter is a fumarate-and-nitrase reductase promoter.

“Exogenous environmental condition(s)” refer to setting(s) or circumstance(s) under which the promoter described herein is induced. In some embodiments, the exogenous environmental conditions are specific to a malignant growth containing cancerous cells, e.g., a tumor. The phrase “exogenous environmental conditions” is meant to refer to the environmental conditions external to the intact (unlysed) engineered microorganism, but endogenous or native to tumor environment or the host subject environment. Thus, “exogenous” and “endogenous” may be used interchangeably to refer to environmental conditions in which the environmental conditions are endogenous to a mammalian body, but external or exogenous to an intact microorganism cell. In some embodiments, the exogenous environmental conditions are low-oxygen, microaerobic, or anaerobic conditions, such as hypoxic and/or necrotic tissues. Some solid tumors are associated with low intracellular and/or extracellular pH; in some embodiments, the exogenous environmental condition is a low-pH environment. In some aspects, bacteria have evolved transcription factors that are capable of sensing oxygen levels. Different signaling pathways may be triggered by different oxygen levels and occur with different kinetics. An “oxygen level-dependent promoter” or “oxygen level-dependent regulatory region” refers to a nucleic acid sequence to which one or more oxygen level-sensing transcription factors is capable of binding, wherein the binding and/or activation of the corresponding transcription factor activates downstream gene expression.

Examples of oxygen level-dependent transcription factors include, but are not limited to, FNR (fumarate and nitrate reductase), ANR, and DNR. Corresponding FNR-responsive promoters, ANR (anaerobic nitrate respiration)-responsive promoters, and DNR (dissimilatory nitrate respiration regulator)-responsive promoters are known in the art (see, e.g., Castiglione et al., 2009; Eiglmeier et al., 1989; Galimand et al., 1991; Hasegawa et al., 1998; Hoeren et al., 1993; Salmon et al., 2003), and non-limiting examples are shown in Table 2.

In a non-limiting example, a promoter (PfnrS) was derived from the E. coli Nissle fumarate and nitrate reductase gene S (fnrS) that is known to be highly expressed under conditions of low or no environmental oxygen (Durand and Storz, 2010; Boysen et al, 2010). The PfnrS promoter is activated under anaerobic conditions by the global transcriptional regulator FNR that is naturally found in Nissle. Under anaerobic conditions, FNR forms a dimer and binds to specific sequences in the promoters of specific genes under its control, thereby activating their expression. However, under aerobic conditions, oxygen reacts with iron-sulfur clusters in FNR dimers and converts them to an inactive form. hi this way, the PfnrS inducible promoter is adopted to modulate the expression of proteins or RNA. PfnrS is used interchangeably in this application as FNRS, fnrs, FNR, P-FNRS promoter and other such related designations to indicate the promoter PfnrS.

TABLE 2
Examples of transcription factors and
responsive genes and regulatory regions

| Transcription | Examples of responsive genes, promoters, |
|---|---|
| Factor | and/or regulatory regions: |
| FNR | nirB, ydfZ, pdhR, focA, ndH, hlyE, narK, |
| | narX, narG, yfiD, tdcD |
| ANR | arcDABC |
| DNR | norb, norC |

“Constitutive promoter” refers to a promoter that is capable of facilitating continuous transcription of a coding sequence or gene under its control and/or to which it is operably linked. Constitutive promoters and variants are well known in the art and non-limiting examples of constitutive promoters are described herein and in International Patent Application PCT/US2017/013072, filed Jan. 11, 2017 and published as WO2017/123675, the contents of which is herein incorporated by reference in its entirety. In some embodiments, such promoters are active in vitro, e.g., under culture, expansion and/or manufacture conditions. In some embodiments, such promoters are active in vivo, e.g., in conditions found in the in vivo environment, e.g., the gut and/or the tumor microenvironment.

As used herein, “stably maintained” or “stable” bacterium or virus is used to refer to a bacterial or viral host cell carrying non-native genetic material, such that the non-native genetic material is retained, expressed, and propagated. The stable bacterium or virus is capable of survival and/or growth in vitro, e.g., in medium, and/or in vivo, e.g., in hypoxic and/or necrotic tissues. For example, the stable bacterium or virus may be a genetically engineered bacterium comprising non-native genetic material, in which the plasmid or chromosome carrying the non-native genetic material is stably maintained in the bacterium or virus, such that the protein encoded by the non-native genetic material can be expressed in the bacterium or virus, and the bacterium or virus is capable of survival and/or growth in vitro and/or in vivo.

As used herein, the terms “modulate” and “treat” and their cognates refer to an amelioration of a cancer, or at least one discernible symptom thereof. In another embodiment, “modulate” and “treat” refer to an amelioration of at least one measurable physical parameter, not necessarily discernible by the patient. In another embodiment, “modulate” and “treat” refer to inhibiting the progression of a cancer, either physically (e.g., stabilization of a discernible symptom), physiologically (e.g., stabilization of a physical parameter), or both. In another embodiment, “modulate” and “treat” refer to slowing the progression or reversing the progression of a cancer. As used herein, “prevent” and its cognates refer to delaying the onset or reducing the risk of acquiring a given cancer.

Those in need of treatment may include individuals already having a particular cancer, as well as those at risk of having, or who may ultimately acquire the cancer. The need for treatment is assessed, for example, by the presence of one or more risk factors associated with the development of a cancer (e.g., alcohol use, tobacco use, obesity, excessive exposure to ultraviolet radiation, high levels of estrogen, family history, genetic susceptibility), the presence or progression of a cancer, or likely receptiveness to treatment of a subject having the cancer. Cancer is caused by genomic instability and high mutation rates within affected cells. Treating cancer may encompass eliminating symptoms associated with the cancer and/or modulating the growth and/or volume of a subject's tumor, and does not necessarily encompass the elimination of the underlying cause of the cancer, e.g., an underlying genetic predisposition.

As used herein, the term “conventional cancer treatment” or “conventional cancer therapy” refers to treatment or therapy that is widely accepted and used by most healthcare professionals. It is different from alternative or complementary therapies, which are not as widely used. Examples of conventional treatment for cancer include surgery, chemotherapy, targeted therapies, radiation therapy, tomotherapy, immunotherapy, cancer vaccines, hormone therapy, hyperthermia, stem cell transplant (peripheral blood, bone marrow, and cord blood transplants), photodynamic therapy, therapy, and blood product donation and transfusion.

As used herein a “pharmaceutical composition” refers to a preparation of at least one microorganism of the disclosure and/or at least one immune modulator, with other components such as a physiologically suitable carrier and/or excipient.

The phrases “physiologically acceptable carrier” and “pharmaceutically acceptable carrier” which may be used interchangeably refer to a carrier or a diluent that does not cause significant irritation to an organism and does not abrogate the biological activity and properties of the administered bacterial or viral compound. An adjuvant is included under these phrases.

The term“excipient” refers to an inert substance added to a pharmaceutical composition to further facilitate administration of an active ingredient. Examples include, but are not limited to, calcium bicarbonate, calcium phosphate, various sugars and types of starch, cellulose derivatives, gelatin, vegetable oils, polyethylene glycols, and surfactants, including, for example, polysorbate 20.

The terms “therapeutically effective dose” and “therapeutically effective amount” are used to refer to an amount of a compound that results in prevention, delay of onset of symptoms, or amelioration of symptoms of a condition, e.g., a cancer. A therapeutically effective amount may, for example, be sufficient to treat, prevent, reduce the severity, delay the onset, and/or reduce the risk of occurrence of one or more symptoms of a disorder associated with cancerous cells. A therapeutically effective amount, as well as a therapeutically effective frequency of administration, can be determined by methods known in the art and discussed below.

In some embodiments, the term “therapeutic molecule” refers to a molecule or a compound that is results in prevention, delay of onset of symptoms, or amelioration of symptoms of a condition, e.g., a cancer. In some embodiments, a therapeutic molecule may be, for example, a cytokine, a chemokine, a single chain antibody, a ligand, a metabolic converter, e.g., arginine, a kynurenine consumer, or an adenosine consumer, a T cell co-stimulatory receptor, a T cell co-stimulatory receptor ligand, an engineered chemotherapy, or a lytic peptide, among others.

The articles “a” and “an,” as used herein, should be understood to mean “at least one,” unless clearly indicated to the contrary.

The phrase “and/or,” when used between elements in a list, is intended to mean either (1) that only a single listed element is present, or (2) that more than one element of the list is present. For example, “A, B, and/or C” indicates that the selection may be A alone; B alone; C alone; A and B; A and C; B and C; or A, B, and C. The phrase “and/or” may be used interchangeably with “at least one of” or “one or more of” the elements in a list.

### Bacteria

In one embodiment, the microorganism may be a bacterium. The bacteria may be administered systemically, orally, locally and/or intratumorally. In some embodiments, the bacteria are capable of targeting cancerous cells, particularly in the hypoxic regions of a tumor, and are administered in combination with, e.g., an immune modulator, e.g., immune stimulator or sustainer provided herein.

In some embodiments, the tumor-targeting microorganism is a bacterium that is naturally capable of directing itself to cancerous cells, necrotic tissues, and/or hypoxic tissues. For example, bacterial colonization of tumors may be achieved without any specific genetic modifications in the bacteria or in the host (Yu et al., 2008). In some embodiments, the tumor-targeting bacterium is a bacterium that is not naturally capable of directing itself to cancerous cells, necrotic tissues, and/or hypoxic tissues, but is genetically engineered to do so. In some embodiments, the bacteria spread hematogenously to reach the targeted tumor(s). Bacterial infection has been linked to tumor regression (Hall, 1998; Nauts and McLaren, 1990), and certain bacterial species have been shown to localize to and lyse necrotic mammalian tumors (Jain and Forbes, 2001). Non-limiting examples of tumor-targeting bacteria are shown in Table 3.

TABLE 3
Bacteria with tumor-targeting capability

| Bacterial Strain | See, e.g., |
|---|---|
| Clostridium novyi-NT | Forbes, Neil S. “Profile of a bacterial tumor killer.” Nature |
| | biotechnology 24.12 (2006): 1484-1485. |
| Bifidobacterium spp | Liu, Sai, et al. “Tumor-targeting bacterial therapy: A potential |
| Streptococcus spp | treatment for oral cancer.” Oncology letters 8.6 (2014): 2359- |
| Caulobacter spp | 2366. |
| Clostridium spp | |
| Escherichia coli MG1655 | Cronin, Michelle, et al. “High resolution in vivo |
| Escherichia coli Nissle | bioluminescent imaging for the study of bacterial tumour |
| Bifidobacterium breve UCC2003 | targeting.” PloS one 7.1 (2012): e30940.; Zhou, et al., Med |
| Salmonella typhimurium | Hypotheses. 2011 April; 76(4): 533-4. doi: |
| | 10.1016/j.mehy.2010.12.010. Epub 2011 Jan. 21; Zhang et al., |
| | Appl Environ Microbiol. 2012 Nov; 78(21): 7603-7610; |
| | Danino et al., Science Translational Medicine, 2015 Vol 7 |
| | Issue 289, pp. 289ra84 |
| Clostridium novyi-NT | Bernardes, Nuno, Ananda M. Chakrabarty, and Arsenio M. |
| Bifidobacterium spp | Fialho. “Engineering of bacterial strains and their products for |
| Mycobacterium bovis | cancer therapy.” Applied microbiology and biotechnology |
| Listeria monocytogenes | 97.12 (2013): 5189-5199. |
| Escherichia coli | |
| Salmonella spp | |
| Salmonella typhimurium | |
| Salmonella choleraesuis | Patyar, S., et al. “Bacteria in cancer therapy: a novel |
| Vibrio cholera | experimental strategy.” J Biomed Sci 17.1 (2010): 21-30. |
| Listeria monocytogenes | |
| Escherichia coli | |
| Bifidobacterium adolescentis | |
| Clostridium acetobutylicum | |
| Salmonella typhimurium | |
| Clostridium histolyticum | |
| Escherichia coli Nissle 1917 | Danino et al. “Programmable probiotics for detection of |
| | cancer in urine.” Sci Transl Med. 2015 May |
| | 27; 7(289): 289ra84 |

In some embodiments, the bacterium which enhances the efficacy of immunotherapy. Recent studies have suggested that the presence of certain types of gut microbes in mice can enhance the anti-tumor effects of cancer immunotherapy without increasing toxic side effects (M. Vetizou et al., “Anticancer immunotherapy by CTLA-4 blockade relies on the gut microbiota,” Science, doi:10.1126/aad1329, 2015; A. Sivan et al., “Commensal Bifidobacterium promotes antitumor immunity and facilitates anti-PD-L1 efficacy,” Science, doi:0.1126/science.aac4255, 2015). Whether the gut microbial species identified in these mouse studies will have the same effect in humans is not clear. Vetizou et al (2015) describe T cell responses specific for Bacteroides thetaiotaomicron or Bacteroides fragilis that were associated with the efficacy of CTLA-4 blockade in mice and in patients. Sivan et al. (2015) illustrate the importance of Bifidobacterium to antitumor immunity and anti-PD-L1 antibody against (PD-1 ligand) efficacy in a mouse model of melanoma.

In some embodiments, the bacteria are Bacteroides. In some embodiments, the bacteria are Bifidobacterium. In some embodiments, the bacteria are Escherichia Coli Nissle. In some embodiments, the bacteria are Clostridium novyi-NT. In some embodiments, the bacteria are Clostridium butyricum miyairi.

In certain embodiments, the microorganisms are obligate anaerobic bacteria. In certain embodiments, the bacteria are facultative anaerobic bacteria. In certain embodiments, the bacteria are aerobic bacteria. In some embodiments, the bacteria are Gram-positive bacteria and lack LPS. In some embodiments, the bacteria are Gram-negative bacteria. In some embodiments, the bacteria are Gram-positive and obligate anaerobic bacteria. In some embodiments, the bacteria are Gram-positive and facultative anaerobic bacteria. In some embodiments, the bacteria are non-pathogenic bacteria. In some embodiments, the bacteria are commensal bacteria. In some embodiments, the bacteria are probiotic bacteria.

Exemplary bacteria include, but are not limited to, Bacillus, Bacteroides, Bifidobacterium, Brevibacteria, Caulobacter, Clostridium, Enterococcus, Escherichia coli, Lactobacillus, Lactococcus, Listeria, Mycobacterium, Saccharomyces, Salmonella, Staphylococcus, Streptococcus, Vibrio, Bacillus coagulans, Bacillus subtilis, Bacteroides fragilis, Bacteroides subtilis, Bacteroides thetaiotaomicron, Bifidobacterium adolescentis, Bifidobacterium bifidum, Bifidobacterium breve UCC2003, Bifidobacterium infantis, Bifidobacterium lactis, Bifidobacterium longum, Clostridium acetobutylicum, Clostridium butyricum, Clostridium butyricum M-55, Clostridium butyricum miyairi, Clostridium cochlearum, Clostridium felsineum, Clostridium histolyticum, Clostridium multifermentans, Clostridium novyi-NT, Clostridium paraputrificum, Clostridium pasteureanum, Clostridium pectinovorum, Clostridium perfringens, Clostridium roseum, Clostridium sporogenes, Clostridium tertium, Clostridium tetani, Clostridium tyrobutyricum, Corynebacterium parvum, Escherichia coli MG1655, Escherichia coli Nissle 1917, Listeria monocytogenes, Mycobacterium bovis, Salmonella choleraesuis, Salmonella typhimurium, Vibrio cholera, and the bacteria shown in Table 3. In certain embodiments, the bacteria are selected from the group consisting of Enterococcus faecium, Lactobacillus acidophilus, Lactobacillus bulgaricus, Lactobacillus casei, Lactobacillus johnsonii, Lactobacillus paracasei, Lactobacillus plantarum, Lactobacillus reuteri, Lactobacillus rhamnosus, Lactococcus lactis, and Saccharomyces boulardii. In certain embodiments, the bacteria are selected from the group consisting of Bacteroides fragilis, Bacteroides thetaiotaomicron, Bacteroides subtilis, Bifidobacterium bifidum, Bifidobacterium infantis, Bifidobacterium lactis, Clostridium butyricum, Escherichia coil Nissle, Lactobacillus acidophilus, Lactobacillus plantarum, Lactobacillus reuteri, and Lactococcus lactis.

In some embodiments, Lactobacillus is used. Lactobacillus casei injected intravenously has been found to accumulate in tumors, which was enhanced through nitroglycerin (NG), a commonly used NO donor, likely due to the role of NO in increasing the blood flow to hypovascular tumors (Fang et al., 2016 (Methods Mol Biol. 2016;1409:9-23. Enhancement of Tumor-Targeted Delivery of Bacteria with Nitroglycerin Involving Augmentation of the EPR Effect).

In some embodiments, the bacteria are obligate anaerobes. In some embodiments, the bacteria are Clostridia. Clostridia are obligate anaerobic bacterium that produce spores and are naturally capable of colonizing and in some cases lysing hypoxic tumors (Groot et al., 2007). In experimental models, Clostridia have been used to deliver pro-drug converting enzymes and enhance radiotherapy (Groot et al., 2007). In some embodiments, the bacteria is selected from the group consisting of Clostridium novyi-NT, Clostridium histolyticium, Clostridium tetani, Clostridium oncolyticum, Clostridium sporogenes, and Clostridium beijerinckii (Liu et al., 2014). In some embodiments, the Clostridium is naturally non-pathogenic. For example, Clostridium oncolyticum is a pathogenic and capable of lysing tumor cells. In alternate embodiments, the Clostridium is naturally pathogenic but modified to reduce or eliminate pathogenicity. For example, Clostridium novyi are naturally pathogenic, and Clostridium novyi-NT are modified to remove lethal toxins. Clostridium novyi-NT and Clostridium sporogenes have been used to deliver single-chain HIF-1α antibodies to treat cancer and is an “excellent tumor colonizing Clostridium strains” (Groot et al., 2007).

In some embodiments, the bacteria facultative anaerobes. In some embodiments, the bacteria are Salmonella, e.g., Salmonella typhimurium. Salmonella are non-spore-forming Gram-negative bacteria that are facultative anaerobes. In some embodiments, the Salmonella are naturally pathogenic but modified to reduce or eliminate pathogenicity. For example, Salmonella typhimurium is modified to remove pathogenic sites (attenuated). In some embodiments, the bacteria are Bifidobacterium. Bifidobacterium are Gram-positive, branched anaerobic bacteria. In some embodiments, the Bifidobacterium is naturally non-pathogenic. In alternate embodiments, the Bifidobacterium is naturally pathogenic but modified to reduce or eliminate pathogenicity. Bifidobacterium and Salmonella have been shown to preferentially target and replicate in the hypoxic and necrotic regions of tumors (Yu et al., 2014).

In some embodiments, the bacteria are Gram-negative bacteria. In some embodiments, the bacteria are E. coli. For example, E. coli Nissle has been shown to preferentially colonize tumor tissue in vivo following either oral or intravenous administration (Zhang et al., 2012 and Danino et al., 2015). E. coli have also been shown to exhibit robust tumor-specific replication (Yu et al., 2008). In some embodiments, the bacteria are Escherichia coli strain Nissle 1917 (E. coli Nissle), a Gram-negative bacterium of the Enterobacteriaceae family that “has evolved into one of the best characterized probiotics” (Ukena et al., 2007). The strain is characterized by its complete harmlessness (Schultz, 2008), and has GRAS (generally recognized as safe) status (Reister et al., 2014, emphasis added).

In some embodiments, the bacteria are administered repeatedly. In some embodiments, the bacteria are administered once.

Further examples of bacteria which are suitable are described in International Patent Publication WO/2014/043593, the contents of which are herein incorporated by reference in its entirety. In some embodiments, such bacteria are mutated to attenuate one or more virulence factors. Other bacteria are described at least in Song et at, Infectious Agents and Cancer, 2018; and Lukasiewicz and Fol, J. Immunol. Research, 2018, Article ID 2397808.

In some embodiments, the bacteria of the disclosure proliferate and colonize a tumor. In some embodiments, colonization persists for several days, several weeks, several months, several years or indefinitely. In some embodiments, the bacteria do not proliferate in the tumor and bacterial counts drop off quickly post injection, e.g., less than a week post injection, until no longer detectable.

### Essential Genes and Auxotrophs

As used herein, the term “essential gene” refers to a gene that is necessary to for cell growth and/or survival. Bacterial essential genes are well known to one of ordinary skill in the art, and can be identified by directed deletion of genes and/or random mutagenesis and screening (see, for example, Zhang and Lin, 2009, DEG 5.0, a database of essential genes in both prokaryotes and eukaryotes, Nucl. Acids Res., 37:D455-D458 and Gerdes et al., Essential genes on metabolic maps, Curr. Opin. Biotechnol., 17(5):448-456, the entire contents of each of which are expressly incorporated herein by reference).

An “essential gene” may be dependent on the circumstances and environment in which an organism lives. For example, a mutation of, modification of, or excision of an essential gene may result in the recombinant bacteria of the disclosure becoming an auxotroph. An auxotrophic modification is intended to cause bacteria to die in the absence of an exogenously added nutrient essential for survival or growth because they lack the gene(s) necessary to produce that essential nutrient.

An auxotrophic modification is intended to cause bacteria to die in the absence of an exogenously added nutrient essential for survival or growth because they lack the gene(s) necessary to produce that essential nutrient. In some embodiments, any of the bacteria described herein also comprise a deletion or mutation in a gene required for cell survival and/or growth. In one embodiment, the essential gene is a DNA synthesis gene, for example, thyA. In another embodiment, the essential gene is a bacterial cell wall synthesis gene, for example, dapA. In yet another embodiment, the essential gene is an amino acid gene, for example, serA or metA. Any gene required for cell survival and/or growth may be targeted, including but not limited to, cysE, glnA, ilvD, leuB, lysA, serA, metA, glyA, hisB, ilvA, pheA, preA, thrC, trpC, tyrA, thyA, uraA, dapA, dapB, dapD, dapE, dapE, flhD, metB, metC, proAB, and thil, as long as the corresponding wild-type gene product is not produced in the bacteria. Exemplary bacterial genes which may be disrupted or deleted to produce an auxotrophic strain as described in International Patent Application PCT/US2017/013072, filed Jan. 11, 2017, published as WO2017/123675, the contents of which is herein incorporated by reference in its entirety. These include, but are not limited to, genes required for oligonucleotide synthesis, amino acid synthesis, and cell wall synthesis. Table 4 lists exemplary bacterial genes which may be disrupted or deleted to produce an auxotrophic strain. These include, but are not limited to, genes required for oligonucleotide synthesis, amino acid synthesis, and cell wall synthesis.

TABLE 4
Non-limiting Examples of Bacterial Genes
Useful for Generation of an Auxotroph

| Amino Acid | Oligonucleotide | Cell Wall |
|---|---|---|
| cysE | thyA | dapA |
| glnA | uraA | dapB |
| ilvD | | dapD |
| leuB | | dapE |
| lysA | | dapF |
| serA | | |
| metA | | |
| glyA | | |
| hisB | | |
| ilvA | | |
| pheA | | |
| proA | | |
| thrC | | |
| trpC | | |
| tyrA | | |

Auxotrophic mutations are useful in some instances in which biocontainment strategies may be required to prevent unintended proliferation of the bacterium in a natural ecosystem. Any auxotrophic mutation in an essential gene described above or known in the art can be useful for this purpose, e.g. DNA synthesis genes, amino acid synthesis genes, or genes for the synthesis of cell wall. Accordingly, in some embodiments, the bacteria comprise modifications, e.g., mutation(s) or deletion(s) in one or more auxotrophic genes, e.g., to prevent growth and proliferation of the bacterium in the natural environment. In some embodiments, the modification may be located in a non-coding region. In some embodiments, the modifications result in attenuation of transcription or translation. In some embodiments, the modifications, e.g., mutations or deletions, result in reduced or no transcription or reduced or no translation of the essential gene. In some embodiments, the modifications, e.g., mutations or deletions, result in transcription and/or translation of a non-functional version of the essential gene. In some embodiments, the modifications, e.g., mutations or deletions result in in truncated transcription or translation of the essential gene, resulting in a truncated polypeptide. In some embodiments, the modification, e.g., mutation is located within the coding region of the gene.

While unable to grow in the natural ecosystem, certain auxotrophic mutations may allow growth and proliferation in the mammalian host administered the bacteria, e.g., in the tumor environment. For example, an essential pathway that is rendered non-functional by the auxotrophic mutation may be complemented by production of the metabolite by the host within the tumor microenvironment. As a result, the bacterium administered to the host can take up the metabolite from the environment and can proliferate and colonize the tumor. Thus, in some embodiments, the auxotrophic gene is an essential gene for the production of a metabolite, which is also produced by the mammalian host in vivo, e.g., in a tumor setting. In some embodiments, metabolite production by the host tumor may allow uptake of the metabolite by the bacterium and permit survival and/or proliferation of the bacterium within the tumor. In some embodiments, bacteria comprising such auxotrophic mutations are capable of proliferating and colonizing the tumor to the same extent as a bacterium of the same subtype which does not carry the auxotrophic mutation.

In some embodiments, the bacteria are capable of colonizing and proliferating in the tumor microenvironment. In some embodiments, the tumor colonizing bacteria comprise one or more auxotrophic mutations. In some embodiments, the tumor colonizing bacteria do not comprise one or more auxotrophic modifications or mutations. In a non-limiting example, greater numbers of bacteria are detected after 24 hours and 72 hours than were originally injected into the subject. In some embodiments, CFUs detected 24 hours post injection are at least about 1 to 2 logs greater than administered. In some embodiments, CFUs detected 24 hours post injection are at least about 2 to 3 logs greater than administered. In some embodiments, CFUs detected 24 hours post injection are at least about 3 to 4 logs greater than administered. In some embodiments, CFUs detected 24 hours post injection are at least about 4 to 5 logs greater than administered. In some embodiments, CFUs detected 24 hours post injection are at least about 5 to 6 logs greater than administered. In some embodiments, CFUs detected 72 hours post injection are at least about 1 to 2 logs greater than administered. In some embodiments, CFUs detected 72 hours post injection are at least about 2 to 3 logs greater than administered. In some embodiments, CFUs detected 72 hours post injection are at least about 3 to 4 logs greater than administered. In some embodiments, CFUs detected 72 hours post injection are at least about 4 to 5 logs greater than administered. In some embodiments, CFUs detected 72 hours post injection are at least about 5 to 6 logs greater than administered. In some embodiments, CFUs can be measured at later time points, such as after at least one week, after at least 2 or more weeks, after at least one month, after at least two or more months post injection.

Non-limiting examples of such auxotrophic genes, which allow proliferation and colonization of the tumor, are thyA and uraA, as shown herein. Accordingly, in some embodiments, the bacteria of the disclosure may comprise an auxotrophic modification, e.g., mutation or deletion, in the thyA gene. In some embodiments, the bacteria of the disclosure may comprise an auxotrophic modification, e.g., mutation or deletion, in the uraA gene. In some embodiments, the bacteria of the disclosure may comprise auxotrophic modification, e.g., mutation or deletion, in the thyA gene and the uraA gene.

Alternatively, the auxotrophic gene is an essential gene for the production of a metabolite which cannot be produced by the host within the tumor, i.e., the auxotrophic mutation is not complemented by production of the metabolite by the host within the tumor microenvironment. As a result, the this mutation may affect the ability of the bacteria to grow and colonize the tumor and bacterial counts decrease over time. This type of auxotrophic mutation can be useful for the modulation of in vivo activity of the immune modulator or duration of activity of the immune modulator, e.g., within a tumor. An example of this method of fine-tuning levels and timing of immune modulator release is described herein using a auxotrophic modification, e.g., mutation, in dapA. Diaminopimelic acid (Dap) is a characteristic component of certain bacterial cell walls, e.g., of gram negative bacteria. Without diaminopimelic acid, bacteria are unable to form proteoglycan, and as such are unable to grow. DapA is not produced by mammalian cells, and therefore no alternate source of DapA is provided in the tumor. As such, a dapA auxotrophy may present a particularly useful strategy to modulate and fine tune timing and extent of bacterial presence in the tumor and/or levels and timing of immune modulator expression and production. Accordingly, in some embodiments, the bacteria of the disclosure comprise an mutation in an essential gene for the production of a metabolite which cannot be produced by the host within the tumor. In some embodiments, the auxotrophic mutation is in a gene which is essential for the production and maintenance of the bacterial cell wall known in the art or described herein, or a mutation in a gene that is essential to another structure that is unique to bacteria and not present in mammalian cells. In some embodiments, bacteria comprising such auxotrophic mutations are capable of proliferating and colonizing the tumor to a substantially lesser extent than a bacterium of the same subtype which does not carry the auxotrophic mutation. Control of bacterial growth (and by extent effector levels) may be further combined with other regulatory strategies, including but not limited to, metabolite or chemically inducible promoters described herein.

In a non-limiting example, lower numbers of bacteria are detected after 24 hours and 72 hours than were originally injected into the subject. In some embodiments, CFUs detected 24 hours post injection are at least about 1 to 2 logs lower than administered. In some embodiments, CFUs detected 24 hours post injection are at least about 2 to 3 logs lower than administered. In some embodiments, CFUs detected 24 hours post injection are at least about 3 to 4 logs lower than administered. In some embodiments, CFUs detected 24 hours post injection are at least about 4 to 5 logs lower than administered. In some embodiments, CFUs detected 24 hours post injection are at least about 5 to 6 logs lower than administered. In some embodiments, CFUs detected 72 hours post injection are at least about 1 to 2 logs lower than administered. In some embodiments, CFUs detected 72 hours post injection are at least about 2 to 3 logs lower than administered. In some embodiments, CFUs detected 72 hours post injection are at least about 3 to 4 logs lower than administered. In some embodiments, CFUs detected 72 hours post injection are at least about 4 to 5 logs lower than administered. In some embodiments, CFUs detected 72 hours post injection are at least about 5 to 6 logs lower than administered. In some embodiments, CFUs can be measured at later time points, such as after at least one week, after at least 2 or more weeks, after at least one month, after at least two or more months post injection.

In some embodiments, the bacteria of the disclosure comprise a auxotrophic modification, e.g., mutation, in dapA. trpE is another auxotrophic mutation described herein. Bacteria carrying this mutation cannot produce tryptophan. In some embodiments, the bacteria comprise auxotrophic mutation(s) in one essential gene. In some embodiments, the bacteria comprise auxotrophic mutation(s) in two essential genes (double auxotrophy). In some embodiments, the bacteria comprise auxotrophic mutation(s) in three or more essential gene(s).

In some embodiments, the bacteria comprise auxotrophic mutation(s) in dapA and thyA. In some embodiments, the bacteria comprise auxotrophic mutation(s) in dapA and uraA. In some embodiments, the bacteria comprise auxotrophic mutation(s) in thyA and uraA. In some embodiments, the bacteria comprise auxotrophic mutation(s) in dapA, thyA and uraA.

In some embodiments, the bacteria comprise auxotrophic mutation(s) in trpE. In some embodiments, the bacteria comprise auxotrophic mutation(s) in trpE and thyA. In some embodiments, the bacteria comprise auxotrophic mutation(s) in trpE and dapA. In some embodiments, the bacteria comprise auxotrophic mutation(s) in trpE and uraA. In some embodiments, the bacteria comprise auxotrophic mutation(s) in trpE, dapA and thyA. In some embodiments, the bacteria comprise auxotrophic mutation(s) in trpE, dapA and uraA. In some embodiments, the bacteria comprise auxotrophic mutation(s) in trpE, thyA and uraA. In some embodiments, the bacteria comprise auxotrophic mutation(s) in trpE, dapA, thyA and uraA.

In another non-limiting example, a conditional auxotroph can be generated. The chromosomal copy of dapA or thyA is knocked out. Another copy of thyA or dapA is introduced, e.g., under control of a low oxygen promoter. Under anaerobic conditions, dapA or thyA—as the case may be—are expressed, and the strain can grow in the absence of clap or thymidine. Under aerobic conditions, dapA or thyA expression is shut off, and the strain cannot grow in the absence of dap or thymidine. Such a strategy can also be employed to allow survival of bacteria under anaerobic conditions, e.g., the gut or conditions of the tumor microenvironment, but prevent survival under aerobic conditions.

In some embodiments, the bacterium of the present disclosure is a synthetic ligand-dependent essential gene (SLiDE) bacterial cell. SLiDE bacterial cells are synthetic auxotrophs with a mutation in one or more essential genes that only grow in the presence of a particular ligand (see Lopez and Anderson “Synthetic Auxotrophs with Ligand-Dependent Essential Genes for a BL21 (DE3 Biosafety Strain,” ACS Synthetic Biology (2015) DOI: 10.1021/acssynbio.5b00085, the entire contents of which are expressly incorporated herein by reference). SLiDE bacterial cells are described in International Patent Application PCT/US2017/013072, filed Jan. 11, 2017, published as WO2017/123675, the contents of which is herein incorporated by reference in its entirety.

### Immune Modulators

### Oncolysis and Activation of an Innate Immune Response

In certain embodiments, the immune modulators(s) of the disclosure generate an innate antitumor immune response. In certain embodiments, the immune modulators(s) generate a local antitumor immune response. In some aspects, the immune modulator is able to activate systemic antitumor immunity against distant cancer cells. In certain embodiments, the immune modulators(s) generate a systemic or adaptive antitumor immune response. In some embodiments, the immune modulators(s) result in long-term immunological memory. Examples of suitable immune modulators(s), e.g., immune initiators and/or immune sustainers are described herein.

In other embodiments, one or more immune modulators may be administered in combination with a microorganism. Alternatively, one or more first immune modulators may be administered in combination with a microorganism and one or more second immune modulators.

Many immune cells found in the tumor microenvironment express pattern recognition receptors (PRRs), which receptors play a key role in the innate immune response through the activation of pro-inflammatory signaling pathways, stimulation of phagocytic responses (macrophages, neutrophils and dendritic cells) or binding to micro-organisms as secreted proteins. PRRs recognize two classes of molecules: (PAMPs), which are associated with microbial pathogens, and damage-associated molecular patterns (DAMPs), which are associated with cell components that are released during cell damage, death stress, or tissue injury. PAMPS are unique to each pathogen and are essential molecular structures required for the pathogens survival, e.g., bacterial cell wall molecules (e.g. lipoprotein), viral capsid proteins, and viral and bacterial DNA. PRRs can identify a variety of microbial pathogens, including bacteria, viruses, parasites, fungi, and protozoa. PRRs are primarily expressed by cells of the innate immune system, e.g., antigen presenting macrophage and dendritic cells, but can also be expressed by other cells (both immune and non-immune cells), and are either localized on the cell surface to detect extracellular pathogens or within the endosomes and cellular matrix where they detect intracellular invading viruses.

Examples of PRRs include Toll-like receptors (TLR), which are type 1 transmembrane receptors that have an extracellular domain which detects infecting pathogens. TLR1, 2, 4, and 6 recognize bacterial lipids, TLR3, 7 and 8 recognize viral RNA, TLR9 recognizes bacterial DNA, and TLR5 and 10 recognize bacterial or parasite proteins. Other examples of PRRs include C-type lectin receptors (CLR), e.g., group I mannose receptors and group II asialoglycoprotein receptors, cytoplasmic (intracellular) PRRs, nucleotide oligomerization (NOD)-like receptors (NLRs), e.g., NOD1 and NOD2, retinoic acid-inducible gene I (RIG-I)-like receptors (RLR), e.g., RIG-I, MDA5, and DDX3, and secreted PRRs, e.g., collectins, pentraxins, ficolins, lipid transferases, peptidoglycan recognition proteins (PGRs) and the leucine-rich repeat receptor (LRR).

PRRs initiate the activation of signaling pathways, such as the NF-kappa B pathway, that stimulates the production of co-stimulatory molecules and pro-inflammatory cytokines, e.g., type I IFNs, IL-6, TNF, and IL-12, which mechanisms play a role in the activation of inflammatory and immune responses mounted against infectious pathogens. Such response triggers the activation of immune cells present in the tumor microenvironment that are involved in the adaptive immune response (e.g., antigen-presenting cells (APCs) such as B cells, DCs, TAMs, and other myeloid derived suppressor cells). Recent evidence indicates that immune mechanisms activated by PAMPs and DAMPs play a role in activating immune responses against tumor cells as well (LeMercier et al., Canc Res, 73:4629-40 (2013); Kim et al., Blood, 119:355-63 (2012)).

Another PRR subfamily are the RIG-I-like receptors(RLRs) which are considered to be sensors of double-stranded viral RNA upon viral infection and which can be targeted for intratumoral immune stimulation. Upon stimulation, for example, upon intratumoral delivery of an oncolytic virus, RLRs trigger the release of type I IFNs by the host cell and result in its death by apoptosis. Such cytokine and tumor-associated antigen (TAA) release also results in the activation of the antitumor immune response. Given that RLRs are endogenously expressed in all tumor types, they are a universal proimmunogenic therapeutic target and of particular relevance in the immune response generated by local delivery of an oncolytic virus.

In some aspects, a bacterial chassis itself may activate one or more of the PRR receptors, e.g., TLRs or RIGI, and stimulate an innate immune response.

In some embodiments, the bacteria are administered intratumorally and 5-FC is administered systemically. In some embodiments, both the bacteria and 5-FC are administered systemically.

In any of these combination embodiments, the bacteria may further comprise an auxotrophic modification, e.g., a mutation or deletion in DapA, ThyA, or both. In any of these embodiments, the bacteria may further comprise a phage modification, e.g., a mutation or deletion, in an endogenous prophage as described herein. In any of these embodiments, the bacteria may further comprise one or more antibiotic resistance circuits.

### Inhibition of Phagocytosis Escape—CD47-SIRPα Pathway

Cancers have the ability to up-regulate the “don't eat me” signal to allow escape from endogenous “eat me” signals that were induced as part of programmed cell death and programmed cell removal, to promote tumor progression.

CD47 is a cell surface molecule implicated in cell migration and T cell and dendritic cell activation. In addition, CD47 functions as an inhibitor of phagocytosis through ligation of signal-regulatory protein alpha (SIRPa) expressed on phagocytes, leading to tyrosine phosphatase activation and inhibition of myosin accumulation at the submembrane assembly site of the phagocytic synapse. As a result, CD47 conveys a “don't eat me signal.” Loss of CD47 leads to homeostatic phagocytosis of aged or damaged cells.

Elevated levels of CD47 expression are observed on multiple human tumor types, allowing tumors to escape the innate immune system through evasion of phagocytosis. This process occurs through binding of CD47 on tumor cells to SIRPα on phagocytes, thus promoting inhibition of phagocytosis and tumor survival.

Anti-CD47 antibodies have demonstrated pre-clinical activity against many different human cancers both in vitro and in mouse xenotransplantation models (Chao et al., Curr Opin Immunol. 2012 April; 24(2): 225-232. The CD47-SIRPα Pathway in Cancer Immune Evasion and Potential Therapeutic Implications, and references therein). In addition to CD47, SIRPα can also be targeted as a therapeutic strategy; for example, anti-SIRPα antibodies administered in vitro caused phagocytosis of tumor cells by macrophages (Chao et al., 2012).

In a third approach, CD47-targeted therapies have been developed using the single 14 kDa CD47 binding domain of human SIRPα (a soluble form without the transmembrane portion) as a competitive antagonist to human CD47 (as described in Weiskopf et al., Engineered SIRPα variants as immunotherapeutic adjuvants to anti-cancer antibodies; Science. 2013 Jul. 5; 341(6141): 10.1126/science.1238856, the contents of which is herein incorporated by reference in its entirety). Because the wild type SIRPα showed relatively low affinity to CD47, mutated SIRPα were generated through in vitro evolution via yeast surface display, which were shown to act as strong binders and antagonists of CD47. These variant include CV1 (consensus variant 1) and high-affinity variant FD6, and Fc fusion proteins of these variants. The amino acid changes leading to the increased affinity are located in the dl domain of human SIRPa. Non-limiting examples of SIRPα variants are also described in WO/2013/109752, the contents of which is herein incorporated by reference in its entirety.

In certain embodiments, the one or more immune modulators inhibit CD47 and/or inhibit SIRPα and/or inhibit or prevent the interaction between CD47 and SIRPα expressed on macrophages. For example, the immune modulator may be an antibody directed against CD47 and/or an antibody directed against SIRPα, e.g. a single-chain antibody against CD47 and/or a single-chain antibody against SIRPα.

In another non-limiting example, the immune modulator may be a competitive antagonist polypeptide comprising the SIRPα CD47 binding domain. Such a competitive antagonist polypeptide can function through competitive binding of CD47, preventing the interaction of CD47 with SIRPα expressed on macrophages.

In some embodiments, the immune modulator may be a wild type form of the SIRPα CD47 binding domain. In some embodiments, the immune modulator may be a mutated or variant form of the SIRPα CD47 binding domain. In some embodiments, the variant form is the CV1 SIRPα variant. In some embodiments, the variant form is the FD6 variant. In some embodiments, the SIRPα variant is a variant described in Weiskopf et al., and/or International Patent Publication WO/2013/109752.

In some embodiments, the immune modulator may be a SIRPα CD47 binding domain or variant thereof fused to a stabilizing polypeptide. In a non-limiting example, the stabilizing polypeptide fused to the wild type SIRPα CD47 binding domain polypeptide is a Fc portion. In some embodiments, the stabilizing polypeptide fused to the wild type SIRPα CD47 binding domain polypeptide is the IgG Fc portion. In some embodiments, the stabilizing polypeptide fused to the wild type SIRPα CD47 binding domain polypeptide is the IgG4 Fc portion.

In some embodiments, the immune modulator may be a mutated or variant form of the SIRPα CD47 binding domain fused to a stabilizing polypeptide. In some embodiments, the variant form fused to the stabilizing polypeptide is the CV1 SIRPα variant. In some embodiments, the variant form fused to the stabilizing polypeptide is the F6 variant. In some embodiments, the SIRPα variant fused to the stabilizing polypeptide is a variant described in Weiskopf et al., and/or International Patent Publication WO/2013/109752. In a non-limiting example, the stabilizing polypeptide fused to the variant SIRPα CD47 binding domain polypeptide is a Fc portion. In some embodiments, the stabilizing polypeptide fused to the variant SIRPα CD47 binding domain polypeptide is the IgG Fc portion. In some embodiments, the stabilizing polypeptide fused to the variant SIRPα CD47 binding domain polypeptide is an IgG4 Fe portion.

In some embodiments, the immune modulator may be an anti-CD47 antibody and/or anti-SIRPα antibody, e.g., a single chain antibody. In some embodiments, the immune modulator may be a competitive antagonist SIRPα CD47 binding domain (WT or mutated to improve CD47 affinity). In some embodiments, the immune modulator may be an anti-CD47 antibody and/or anti-SIRPα antibody, e.g., a single chain antibody. In any of these embodiments, the microorganisms may also be administered with one or more immune modulators that are capable of stimulating Fc-mediated functions such as ADCC, and/or M-CSF and/or GM-CSF, resulting in a blockade of phagocytosis inhibition.

The immune modulator may be any suitable anti-CD47 antibody, anti-SIRPα antibody or competitive SIRPα CD47 binding domain polypeptide (wild type or mutated variant with improved CD47 binding affinity) for the inhibition or prevention of the CD47-SIRPα interaction.

In any of these embodiments, the SIRPα or variants thereof or anti-CD47 polypeptides may be combined with one or more STING agonists, as described herein.

In any of these combination embodiments, the bacteria may comprise an auxotrophic modification, e.g., a mutation or deletion in DapA, ThyA, or both. In any of these embodiments, the bacteria may further comprise a phage modification, e.g., a mutation or deletion, in an endogenous prophage as described herein.

### Activation of Antigen Presenting Cells

### STING Agonists

Stimulator of interferon genes (STING) protein was shown to be a critical mediator of the signaling triggered by cytosolic nucleic acid derived from DNA viruses, bacteria, and tumor-derived DNA. The ability of STING to induce type I interferon production lead to studies in the context of antitumor immune response, and as a result, STING has emerged to be a potentially potent target in anti-tumor immunotherapies. A large part of the antitumor effects caused by STING activation may depend upon production of IFN-β by APCs and improved antigen presentation by these cells, which promotes CD8+ T cell priming against tumor-associated antigens. However, STING protein is also expressed broadly in a variety of cell types including myeloid-derived suppressor cells (MDSCs) and cancer cells themselves, in which the function of the pathway has not yet been well characterized (Sokolowska, O. & Nowis, D; STING Signaling in Cancer Cells: Important or Not?; Archivum Immunologiae et Therapiae Experimentalis; Arch. Immunol. Ther. Exp. (2018) 66: 125).

Stimulator of interferon genes (STING), also known as transmembrane protein 173 (TMEM173), mediator of interferon regulatory factor 3 activation (MITA), MPYS or endoplasmic reticulum interferon stimulator (ERIS), is a dimeric protein which is mainly expressed in macrophages, T cells, dendritic cells, endothelial cells, and certain fibroblasts and epithelial cells. STING plays an important role in the innate immune response—mice lacking STING are viable though prone to lethal infection following exposure to a variety of microbes. STING functions as a cytosolic receptor for the second messengers in the form of cytosolic cyclic dinucleotides (CDNs), such as cGAMP and the bacterial second messengers c-di-GMP and c-di-AMP. Upon stimulation by the CDN a conformational change in STING occurs. STING translocates from the ER to the Golgi apparatus and its carboxyterminus is liberated, This leads to the activation of TBK1 (TANK-binding kinase 1)/IRF3 (interferon regulatory factor 3), NF-κB, and STAT6 signal transduction pathways, and thereby promoting type I interferon and proinflammatory cytokine responses. CDNs include canonical cyclic di-GMP (c[G(30-50)pG(30-50)p] or cyclic di-AMP or cyclic GAMP (cGMP-AMP) (Barber, STING-dependent cytosolic DNA sensing pathways; Trends Immunol. 2014 February; 35(2):88-93).

CDNs can be exogenously (i.e., bacterially) and/or endogenously produced (i.e., within the host by a host enzyme upon exposure to dsDNA). STING is able to recognize various bacterial second messenger molecules cyclic diguanylate monophosphate (c-di-GMP) and cyclic diadenylate monophosphate (c-di-AMP), which triggers innate immune signaling response (Ma et al., . The cGAS-STING Defense Pathway and Its Counteraction by Viruses ; Cell Host & Microbe 19, Feb. 10, 2016). Additionally cyclic GMPAMP (cGAMP) can also bind to STING and result inactivation of IRF3 and β-interferon production. Both 3′5′-3′5′ cGAMP (3′3′ cGAMP) produced by Vibrio cholerae, and the metazoan secondary messenger cyclic [G(2′,5′)pA(3′5′)] (2′3′ cGAMP), could activate the innate immune response through STING pathway (Yi et al., Single Nucleotide Polymorphisms of Human STING Can Affect Innate Immune Response to Cyclic Dinucleotides; PLOS One (2013). 8(10)e77846, an references therein). Bacterial and metazoan (e.g., human) c-di-GAMP synthases (cGAS) utilizes GTP and ATP to generate cGAMP capable of STING activation. In contrast to prokaryotic CDNs, which have two canonical 30-50 phosphodiester linkages, the human cGAS product contains a unique 20-50 bond resulting in a mixed linkage cyclic GMP-AMP molecule, denoted as 2′,3′ cGAMP (as described in (Kranzusch et al., Ancient Origin of cGAS-STING Reveals Mechanism of Universal 2′,3′ cGAMP Signaling; Molecular Cell 59, 891-903, Sep. 17, 2015 and references therein). The bacterium Vibrio cholerae encodes an enzyme called DncV that is a structural homolog of cGAS and synthesizes a related second messenger with canonical 3′-5′ bonds (3′,3′ cGAMP).

Components of the stimulator of interferon genes (STING) pathway plays an important role in the detection of tumor cells by the immune system. In preclinical studies, cyclic dinucleotides(CDN), naturally occurring or rationally designed synthetic derivatives, are able to promote an aggressive antitumor response. For example, when co-formulated with an irradiated GM-CSF-secreting whole-cell vaccine in the form of STINGVAX, synthetic CDNs increased the antitumor efficacy and STINGVAX combined with PD-1 blockade induced regression of established tumors (Fu et al., STING agonist formulated cancer vaccines can cure established tumors resistant to PD-1 blockade; Sci Transl Med. 2015 Apr. 15; 7(283): 283ra52). In another example, Smith et al. conducted a study showing that STING agonists may augment CAR T therapy by stimulating the immune response to eliminate tumor cells that are not recognized by the adoptively transferred lymphocytes and thereby improve the effectiveness of CAR T cell therapy (Smith et al., Biopolymers co-delivering engineered T cells and STING agonists can eliminate heterogeneous tumors; J Clin Invest. 2017 Jun 1;127(6):2176-2191).

In some embodiments, the immune modulator is a STING agonist. Non limiting examples of STING agonists include 3′3′ cGAMP, 2′3′cGAMP, 2′2′-cGAMP, 2′2′-cGAMP VacciGrade™ (Cyclic [G(2′,5′)pA(2′,5′)p]), 2′3′-cGAMP, 2′3′ -cGAMP VacciGrade™ (Cyclic [G(2′,5′)pA(3′,5′)p]), 2′3′-cGAM(PS)2 (Rp/Sp), 3′3′-cGAMP, 3′3′-cGAMP VacciGrade™ (Cyclic [G(3′,5′)pA(3′,5′)p]) , c-di-AMP, c-di-AMP VacciGradeTM (Cyclic diadenylate monophosphate Th1/Th2 response), 2′3′-c-di-AMP, 2′3′ -c-di-AM(PS)2 (Rp,Rp) (Bisphosphorothioate analog of c-di-AMP, Rp isomers), 2′3′ -c-di-AM(PS)2 (Rp,Rp) VacciGrade™, c-di-GMP, c-di-GMP VacciGrade™, 2′3′-c-di-GMP, and c-di-IMP. CD40

CD40 is a costimulatory protein found on antigen presenting cells and is required for their activation. The binding of CD154 (CD40L) on T helper cells to CD40 activates antigen presenting cells and induces a variety of downstream immunostimulatory effects. In some embodiments, the immune modulator is an agonist of CD40, for example, an agonist selected from an agonistic anti-CD40 antibody, agonistic anti-CD40 antibody fragment, CD40 ligand (CD40L) polypeptide, and CD40L polypeptide fragment.

### GMCSF

Granulocyte-macrophage colony-stimulating factor (GM-CSF), also known as colony stimulating factor 2 (CSF2), is a monomeric glycoprotein secreted by macrophages, T cells, mast cells, NK cells, endothelial cells and fibroblasts. GM-CSF is a white blood cell growth factor that functions as a cytokine, facilitating the development of the immune system and promoting defense against infections. For example, GM-CSF stimulates stem cells to produce granulocytes (neutrophils, eosinophils, and basophils) and monocytes, which monocytes exit the circulation and migrate into tissue, whereupon they mature into macrophages and dendritic cells. GM-CSF is part of the immune/inflammatory cascade, by which activation of a small number of macrophages rapidly lead to an increase in their numbers, a process which is crucial for fighting infection. GM-CSF signals via the signal transducer and activator of transcription, STATS or via STAT3 (which activates macrophages).

In some embodiments, the immune modulator modulates dendritic cell activation. In some embodiments, the immune modulator is GM-CSF.

### Activation and Priming of Effector Immune Cells (Immune Stimulators) Cytokines and Cytokine Receptors

CD4 (4) is a glycoprotein found on the surface of immune cells such as cells, monocytes, macrophages, and dendritic cells. CD4+ T helper cells are white blood cells that function to send signals to other types of immune cells, thereby assisting other immune cells in immunologic processes, including maturation of B cells Into plasma cells and memory B cells, and activation of cytotoxic T cells and macrophages. T helper cells become activated when they are presented with peptide antigens by MHC class II molecules, which are expressed on the surface of antigen-presenting cells (APCs). Once activated, T helper cells divide and secrete cytokines that regulate or assist in the active immune response. T helper cells can differentiate into one of several subtypes, including TH1, TH2, TH3, TH17, TH9, or TFH cells, which secrete different cytokines to facilitate different types of immune responses.

Cytotoxic T cells (TC cells, or CTLs) destroy virus-infected cells and tumor cells, and are also implicated in transplant rejection. These cells are also known as CD8+ T cells since they express the CD8 glycoprotein at their surfaces. Cytotoxic T cells recognize their targets by binding to antigen associated with MHC class I molecules, which are present on the surface of all nucleated cells.

In some embodiments, the immune modulator modulates one or more T effector cells, e.g., CD4+ cell and/or CD8+ cell. In some embodiments, the immune modulator that activate, stimulate, and/or induce the differentiation of one or more T effector cells, e.g., CD4+ and/or CD8+ cells. In some embodiments, the immune modulator is a cytokine that activates, stimulates, and/or induces the differentiation of a T effector cell, e.g., CD4+ and/or CD8+ cells. In some embodiments, the cytokine is selected from IL-2, IL-15, IL-12, IL-7, IL-21, IL-18, TNF, and IFN-gamma.

As used herein, the term “cytokines” includes fusion proteins which comprise one or more cytokines, which are fused through a peptide linked to another cytokine or other immune modulatory molecule. Examples include but are not limited to IL-12 and IL-15 fusion proteins. In general, all agonists and antagonists described herein may be fused to another polypeptide of interest through a peptide linker, to improve or alter their function. Non-limiting examples of such fusion proteins include one or more cytokine polypeptides operably linked to an antibody polypeptide, wherein the antibody recognizes a tumor-specific antigen, thereby bringing the cytokine(s) into proximity with the tumor.

Interleukin 12 (IL-12) is a cytokine, the actions of which create an interconnection between the innate and adaptive immunity. IL-12 is secreted by a number of immune cells, including activated dendritic cells, monocytes, macrophages, and neutrophils, as well as other cell types. IL-12 is a heterodimeric protein (IL-12-p70; IL-12-p35/p40) consisting of p35 and p40 subunits, and binds to a receptor composed of two subunits, IL-12R-β1 and IL-12R-β2. IL-12 receptor is expressed constitutively or inducibly on a number of immune cells, including NK cells, T, and B lymphocytes. Upon binding of IL-12, the receptor is activated and downstream signaling through the JAK/STAT pathway initiated, resulting in the cellular response to IL-12. IL-12 acts by increasing the production of IFN-γ, which is the most potent mediator of IL-12 actions, from NK and T cells. In addition, IL-12 promotes growth and cytotoxicity of activated NK cells, CD8+ and CD4+ T cells, and shifts the differentiation of CD4+ Th0 cells toward the Th1 phenotype. Further, IL-12 enhances of antibody-dependent cellular cytotoxicity (ADCC) against tumor cells and the induction of IgG and suppression of IgE production from B cells. In addition, IL-12 also plays a role in reprogramming of myeloid-derived suppressor cells, directs the Th1-type immune response and helps increase expression of MHC class I molecules (e.g., reviewed in Waldmann et al., Cancer Immunol Res March 2015 3; 219).

Thus, in some embodiments, the immune modulator is IL-12. In some embodiments, the IL-12 comprises the p35 and p40 subunits. In some embodiments, the interleukin-12 monomer subunits (IL-12A (p35) and IL-12B (p40)) are covalently linked by a linker. In some embodiments, the linker is a serine glycine rich linker. In one embodiment, the 15 amino acid linker of ‘GGGGSGGGGSGGGGS’ (SEQ ID NO: 1247) is inserted between two monomer subunits (IL-12A (p35) and IL-12B (p40) to produce a forced dimer human IL-12 (diIL-12) fusion protein.

IL-15 displays pleiotropic functions in homeostasis of both innate and adaptive immune system and binds to IL-15 receptor, a heterotrimeric receptor composed of three subunits. The alpha subunit is specific for IL-15, while beta (CD122) and gamma (CD132) subunits are shared with the IL-2 receptor, and allow shared signaling through the JAK/STAT pathways. IL-15 is produced by several cell types, including dendritic cells, monocytes and macrophages. Co-expression of IL-15Rα and IL-15 produced in the same cell, allows intracellular binding of IL-15 to IL-15Rα, which is then shuttled to the cell surface as a complex. Once on the cell surface, then, the IL-15Rα of these cells is able to trans-present IL-15 to IL-15Rβ-γc of CD8 T cells, NK cells, and NK-T cells, which do not express IL-15, inducing the formation of the so-called immunological synapse. Murine and human IL-15Rα, exists both in membrane bound, and also in a soluble form. Soluble IL-15Rα (sIL-15Rα) is constitutively generated from the transmembrane receptor through proteolytic cleavage.

IL-15 is critical for lymphoid development and peripheral maintenance of innate immune cells and immunological memory of T cells, in particular natural killer (NK) and CD8+ T cell populations. In contrast to IL-2, IL-15 does not promote the maintenance of Tregs and furthermore, IL-15 has been shown to protect effector T cells from IL-2-mediated activation-induced cell death.

Consequently, delivery of IL-15 is considered a promising strategy for long-term anti-tumor immunity. In a first-in-human clinical trial of recombinant human IL-15, a 10-fold expansion of NK cells and significantly increased the proliferation of γδT cells and CD8+ T cells was observed upon treatment. In addition, IL-15 superagonists containing cytokine-receptor fusion complexes have been developed and are evaluated to increase the length of the response. These include the L-15 N72D superagonist/IL-15RαSushi-Fc fusion complex (IL-15SA/IL-15RαSu-Fc; ALT-803) (Kim et al., 2016 IL-15 superagonist/IL-15RαSushi-Fc fusion complex (IL-15SA/IL-15RαSu-Fc; ALT-803) markedly enhances specific subpopulations of NK and memory CD8+ T cells, and mediates potent anti-tumor activity against murine breast and colon carcinomas).

Thus, in some embodiments, the immune modulator is IL-15.

The biological activity of IL-15 is greatly improved by pre-associating IL-15 with a fusion protein IL-15Rα-Fc or by direct fusion with the sushi domain of IL-15Rα (hyper-IL-15) to mimic trans-presentation of IL-15 by cell-associated IL-15Rα. IL-15, either administrated alone or as a complex with IL-15Ra, exhibits potent antitumor activities in animal models (Cheng et al., Immunotherapy of metastatic and autochthonous liver cancer with IL-15/IL-15Ra fusion protein; Oncoimmunology. 2014; 3(11): e963409, and references therein).

In some embodiments, the immune modulator is IL-15. In some embodiments, the immune modulator is IL-15Ra.

Interferon gamma (IFNγ or type II interferon), is a cytokine that is critical for innate and adaptive immunity against viral, some bacterial and protozoal infections. IFNγ activates macrophages and induces Class II major histocompatibility complex (MHC) molecule expression. IFNγ can inhibit viral replication and has immunostimulatory and immunomodulatory effects in the immune system. IFNγ is produced predominantly by natural killer (NK) and natural killer T (NKT) cells as part of the innate immune response, and by CD4 Th1 and CD8 cytotoxic T lymphocyte (CTL) effector T cells. Once antigen-specific immunity develops IFNγ is secreted by T helper cells (specifically, Th1 cells), cytotoxic T cells (TC cells) and NK cells only. It has numerous immunostimulatory effects and plays several different roles in the immune system, including the promotion of NK cell activity, increased antigen presentation and lysosome activity of macrophages, activation of inducible Nitric Oxide Synthase iNOS, production of certain IgGs from activated plasma B cells, promotion of Thi differentiation that leads to cellular immunity. It can also cause normal cells to increase expression of class I MHC molecules as well as class II MHC on antigen-presenting cells, promote adhesion and binding relating to leukocyte migration, and is involved in granuloma formation through the activation of macrophages so that they become more powerful in killing intracellular organisms. Thus, in one embodiment, the immune modulator is IFN-gamma.

Interleukin-18 (IL-18, also known as interferon-gamma inducing factor) is a proinflammatory cytokine that belongs to the IL-1 superfamily and is produced by macrophages and other cells. IL-18 binds to the interleukin-18 receptor, and together with IL-12 it induces cell-mediated immunity following infection with microbial products like lipopolysaccharide (LPS). Upon stimulation with IL-18, natural killer (NK) cells and certain T helper type 1 cells release interferon-γ (IFN-γ) or type II interferon, which plays a role in activating the macrophages and other immune cells. IL-18 is also able to induce severe inflammatory reactions. Thus, in some embodiments, the immune modulator is IL-18.

Interleukin-2 (IL-2) is cytokine that regulates the activities of white blood cells (leukocytes, often lymphocytes). IL-2 is part of the body's natural response to microbial infection, and in discriminating between foreign (“non-self”) and “self”. IL-2 mediates its effects by binding to IL-2 receptors, which are expressed by lymphocytes. IL-2 is a member of a cytokine family, which also includes IL-4, IL-7, IL-9, IL-15 and IL-21. IL-2 signals through the IL-2 receptor, a complex consisting of alpha, beta and gamma sub-units. The gamma sub-unit is shared by all members of this family of cytokine receptors. IL-2 promotes the differentiation of T cells into effector T cells and into memory T cells when the initial T cell is stimulated by an antigen. Through its role in the development of T cell immunologic memory, which depends upon the expansion of the number and function of antigen-selected T cell clones, it also has a key role in cell-mediated immunity. IL-2 has been approved by the Food and Drug Administration (FDA) and in several European countries for the treatment of cancers (malignant melanoma, renal cell cancer). IL-2 is also used to treat melanoma metastases and has a high complete response rate. Thus, in some embodiments, the immune modulator is IL-2.

Interleukin-21 is a cytokine that has potent regulatory effects on certain cells of the immune system, including natural killer(NK) cells and cytotoxic T cells. IL-21 induces cell division/proliferation in its these cells. IL-21 is expressed in activated human CD4+ T cells but not in most other tissues. In addition, IL-21 expression is up-regulated in Th2 and Th17 subsets of T helper cells. IL-21 is also expressed in NK T cells regulating the function of these cells. When bound to IL-21, the IL-21 receptor acts through the Jak/STAT pathway, utilizing Jakl and Jak3 and a STAT3 homodimer to activate its target genes. IL-21 has been shown to modulate the differentiation programming of human T cells by enriching for a population of memory-type CTL with a unique CD28+ CD127hi CD45RO+ phenotype with IL-2 producing capacity. IL-21 also has anti-tumor effects through continued and increased CD8+ cell response to achieve enduring tumor immunity. IL-21 has been approved for Phase 1 clinical trials in metastatic melanoma (MM) and renal cell carcinoma (RCC) patients. Thus, in some embodiments, the immune modulator is IL-21.

Tumor necrosis factor (TNF) (also known as cachectin or TNF alpha) is a cytokine that can cause cytolysis of certain tumor cell lines and can stimulate cell proliferation and induce cell differentiation under certain conditions. TNF is involved in systemic inflammation and is one of the cytokines that make up the acute phase reaction. It is produced chiefly by activated macrophages, although it can be produced by many other cell types such as CD4+ lymphocytes, NK cells, neutrophils, mast cells, eosinophils, and neurons. The primary role of TNF is in the regulation of immune cells.

TNF can bind two receptors, TNFR1 (TNF receptor type 1; CD120a; p55/60) and TNFR2 (TNF receptor type 2; CD120b; p75/80). TNFR1 is expressed in most tissues, and can be fully activated by both the membrane-bound and soluble trimeric forms of TNF, whereas TNFR2 is found only in cells of the immune system, and respond to the membrane-bound form of the TNF homotrimer. Upon binding to its receptor, TNF can activate NF-κB and MAPK pathways which mediate the transcription of numerous proteins and mediate several pathways involved in cell differentiation and proliferation, including those pathways involved in the inflammatory response. TNF also regulates pathways that induce cell apoptosis. Thus, in some embodiments, immune modulator modulates dendritic cell activation. In some embodiments, the immune modulator is TNF.

In some embodiments, the TNF is capable of increasing CCR7 expression on dendritic cells and/or macrophages.

In some embodiments, the TNFα is capable of activating the NFkappaB pathway, e.g., in cells with TNF receptor. In some embodiments, the TNFα is capable of inducing IkappaBalpha degradation. In some embodiments, TNFα is causes IkappaBalpha degradation.

In some embodiments, the immune modulator may be any one or more of the described IL-2, IL-15, IL-12, IL-7, IL-21, IL-18, TNF, and IFN-gamma.

In any of these combination embodiments, the bacteria administered with the immune modulator may further comprise an auxotrophic modification, e.g., a mutation or deletion in DapA, ThyA, or both. In any of these embodiments, the bacteria may further comprise a phage modification, e.g., a mutation or deletion, in an endogenous prophage as described herein.

### Co-Stimulatory Molecules

Glucocorticoid-induced tumor necrosis factor receptor (TNFR) -related receptor (GITR, TNFR18) is a type I transmembrane protein and a member of the TNFR superfamily.1 GITR is expressed at high levels, predominantly, on CD25+ CD4+ regulatory T (Treg) cells, but it is also constitutively expressed at low levels on conventional CD25-CD4+ and CD8+ T cells and is rapidly upregulated after activation. In vitro studies using an agonistic anti-GITR monoclonal antibody (mAb; DTA-1)2,6,7 or GITRL transfectants and soluble GITRL5,8,9 have shown that the GITR-GITRL pathway induces positive costimulatory signals leading to the activation of CD4+ and CD8+ effector T cells (as well as Treg cells, despite their opposing effector functions) (Piao et al., (2009) Enhancement of T-cell-mediated anti-tumor immunity via the ectopically expressed glucocorticoid-induced tumor necrosis factor receptor-related receptor ligand (GITRL) on tumors; Immunology, 127, 489-499, and references therein). In some embodiments, the effector or immune modulator, is an agonist of GITR, for example, an agonist selected from agonistic anti-GITR antibody, agonistic anti-GITR antibody fragment, GITR ligand polypeptide (GITRL), and GITRL polypeptide fragment.

Thus, in some embodiments, the immune modulator is an agonistic anti-GITR antibody or fragment thereof, or a GITR ligand polypeptide or fragment thereof. Thus, in some embodiments, the immune modulator is an agonistic anti-GITR antibody or fragment thereof, or a GITR ligand polypeptide or fragment thereof. In some embodiments, the immune modulator is anti-GITR antibody or fragment thereof, or a GITR ligand polypeptide or fragment thereof.

As GITR functions to promote T-cell proliferation and T-cell survival in activated T cells, GITR agonism may be advantageously combined with a second modality capable of initiating a T cell response (immune initiator), including but not limited to an innate immune stimulator, such as a STING agonist, as described herein.

CD137 or 4-1BB is a type 2 transmembrane glycoprotein belonging to the TNF superfamily, which is expressed and has a co-stimulatory activity on activated T Lymphocytes (e.g., CD8+ and CD4+ cells). It has been shown to enhance T cell proliferation, IL-2 secretion survival and cytolytic activity. In some embodiments, the immune modulator is an agonist of CD137 (4-1BB), for example, an agonist selected from an agonistic anti-CD137 antibody or fragment thereof, or a CD137 ligand polypeptide or fragment thereof.

Thus, in some embodiments, the immune modulator is an agonistic anti-CD137 antibody or fragment thereof, or a CD ligand polypeptide or fragment thereof.

CD137 (4-1BB) is expressed on activated mouse and human CD8+ and CD4+ T cells. It is a member of the TNFR family and mediates costimulatory and antiapoptotic functions, promoting T-cell proliferation and T-cell survival. CD137 has been reported to be up-regulated-depending on the T-cell stimulus—from 12 hours to up to 5 days after stimulation (Wolff et al., Activation-induced expression of CD137 permits detection, isolation, and expansion of the full repertoire of CD8 T cells responding to antigen without requiring knowledge of epitope specificities; BLOOD, 1 July 2007 VOL. 110, NUMBER 1, and references therein). Accordingly CD137 (4-1BB) agonism may be advantageously combined with a second modality capable of initiating a T cell response (immune initiator), including but not limited to an innate immune stimulator (immune initiator). Exemplary innate immune stimulators (immune initiators) are described herein.

OX40, or CD134, is a T-cell receptor involved in preserving the survival of T cells and subsequently increasing cytokine production. OX40 has a critical role in the maintenance of an immune response and a memory response due to its ability to enhance survival. It also plays a significant role in both Th1 and Th2 mediated reactions. In some embodiments, the immune modulator is an agonist of OX40, for example, an agonist selected from an agonistic anti-OX40 antibody or fragment thereof, or an OX40 ligand (OX40L) or fragment thereof.

Recently, the combination of unmethylated CG-enriched oligodeoxynucleotide (CpG)—a Toll-like receptor 9 (TLR9) ligand—and anti-OX40 antibody injected locally into one site of a tumor was found to synergistically trigger a T cell immune response locally that then attacks cancer throughout the body at distal sites (Sagiv-Barfi et al., Eradication of spontaneous malignancy by local immunotherapy; Sci. Transl. Med. 10, eaan4488 (2018)). Unmethylated CG-enriched oligodeoxynucleotides (CpG) activate TLR9 , a component of the innate immune system. Accordingly other mechanisms of activation the immune system may produce similar results in combination with an agonistic OX40 antibody, including but not limited to bacteria and an innate immune stimulator (immune initiator).

CD28 is one of the proteins expressed on T cells that provide co-stimulatory signals required for T cell activation and survival. In some embodiments, the immune modulator is an agonist of CD28, for example, an agonist selected from agonistic anti-CD28 antibody, agonistic anti-CD28 antibody fragment, CD80 (B7.1) polypeptide or polypeptide fragment thereof, and CD86 (B7.2) polypeptide or polypeptide fragment thereof.

ICOS is an inducible T-cell co-stimulator structurally and functionally related to CD28. In some embodiments, the immune modulator is an agonist of ICOS, for example, an agonist selected from an agonistic anti-ICOS antibody or fragment thereof, or ICOS ligand polypeptide or fragment thereof.

CD226 is a glycoprotein expressed on the surface of natural killer cells, platelets, monocytes, and a subset of T cells (e.g., CD8+ and CD4+ cells), which mediates cellular adhesion to other cells bearing its ligands, CD112 and CD155. Among other things, it is involved in immune synapse formation and triggers Natural Killer (NK) cell activation. In some embodiments, the immune modulator is an agonist of CD226, for example, an agonist selected from agonistic anti-CD226 antibody or fragment thereof, CD112 or CD155 polypeptide or fragments thereof.

In any of these embodiments, the agonistic antibody may be a human antibody or humanized antibody and may comprise different isotypes, e.g., human IgG1, IgG2, IgG3 and IgG4's. Also, the antibody may comprise a constant region that is modified to increase or decrease an effector function such as FcR binding, FcRn binding, complement function, glycosylation, C1q binding; complement dependent cytotoxicity (CDC); Fc receptor binding; antibody-dependent cell-mediated cytotoxicity (ADCC); phagocytosis; down-regulation of cell surface receptors (e.g. B cell receptor; BCR). In any of these embodiments, the antibody may be a single chain antibody or a single chain antibody fragment.

In any of these combination embodiments, the bacteria administered in combination with the immune modulator may comprise an auxotrophic modification, e.g., a mutation or deletion in DapA, ThyA, or both. In any of these embodiments, the bacteria may further comprise a phage modification, e.g., a mutation or deletion, in an endogenous prophage as described herein.

### Elimination (Reversal) of Local Immune Suppression

Tumor cells often escape destruction by producing signals that interfere with antigen presentation or maturation of dendritic cells, causing their precursors to mature into immunosuppressive cell types instead. Therefore, the local delivery of one or more immune modulators that prevent or inhibit the activities of immunomodulatory molecules involved in initiating, promoting and/or maintaining immunosuppression at the tumor site, alone or in combination with one or more other immune modulators, provides a therapeutic benefit.

### Immune Checkpoint Inhibitors

In some embodiments, the immune modulator is an inhibitor of an immune suppressor molecule, for example, an inhibitor of an immune checkpoint molecule. The immune checkpoint molecule to be inhibited can be any known or later discovered immune checkpoint molecule or other immune suppressor molecule. In some embodiments, the immune checkpoint molecule, or other immune suppressor molecule, to be inhibited is selected from CTLA-4, PD-1, PD-L1, PD-L2, TIGIT, VISTA, LAG-3, TIM1, TIM3, CEACAM1, LAIR-1, HVEM, BTLA, CD160, CD200, CD200R, CD39, CD73, B7-H3, B7-H4, IDO, TDO, KIR, and A2aR. In certain aspects, the present disclosure provides a microorganism, e.g., bacteria, in combination with one or more immune modulators that inhibit an immune checkpoint or other immune suppressor molecule.

In some embodiments, the compositions are capable of reducing cancerous cell proliferation, tumor growth, and/or tumor volume. In some embodiments, the bacterium targets a cancer or tumor cell.

In some embodiments, the immune modulator is a CTLA-4 inhibitor, for example, an antibody directed against CTLA-4. In any of these embodiments, the anti-CTLA-4 antibody may be a single-chain anti-CTLA-4 antibody. In some embodiments, the immune modulator is a PD-1 inhibitor, for example, an antibody directed against PD-1 or PD-Ll. In any of these embodiments, the anti-PD-1 or PD-L1 antibody may be a single-chain anti-PD-1 antibody. In some embodiments, the immune modulator is an inhibitor selected from CTLA-4, PD-1, PD-L1, PD-L2, TIGIT, VISTA, LAG-3, TIM1, TIM3, CEACAM1, LAIR-1, HVEM, BTLA, CD160, CD200, CD200R, CD39, CD73, B7-H3, B7-H4, IDO, TDO, KIR, and A2aR inhibitors, e.g., an antibody directed against any of the listed immune checkpoints or other suppressor molecules. Examples of such checkpoint inhibitor molecules are described e.g., in International Patent Application PCT/US2017/013072, filed Jan. 11, 2017, published as WO2017/123675, and PCT/US2018/012698, filed Jan. 1, 2018, the contents of each of which is herein incorporated by reference in its entirety. In any of these embodiments, the antibody may be a single-chain antibody. In some embodiments, the immune modulator is administered locally, e.g., via intratumoral injection.

Exemplary heavy and light chain amino acid sequences for use in constructing single-chain anti-CTLA-4 antibodies are shown are described herein (e.g., SEQ ID NO: 761, SEQ ID NO: 762, SEQ ID NO: 763, SEQ ID NO: 764).

Exemplary heavy and light chain amino acid sequences for use in constructing single-chain anti-PD-1 antibodies include SEQ ID NO: 1, SEQ ID NO: 2, SEQ ID NO: 3, and/or SEQ ID NO: 4.

### Immuno-Metabolism and Metabolic Converters

### Tryptophan and Kynurenine

T regulatory cells, or Tregs, are a subpopulation of T cells that modulate the immune system by preventing excessive immune reactions, maintaining tolerance to self-antigens, and abrogating autoimmunity. Tregs suppress the immune responses of other cells, for example, shutting down immune responses after they have successfully eliminated invading organisms. These cells generally suppress or downregulate induction and proliferation of effector T cells. There are different sub-populations of regulatory T cells, including those that express CD4, CD25, and Foxp3 (CD4+CD25+ regulatory T cells). Tregs are key to dampening effector T cell responses, and therefore represent one of the main obstacles to effective anti-tumor response and the failure of current therapies that rely on induction or potentiation of anti-tumor responses. Thus, in certain embodiments, the bacteria of the present disclosure can be administered in combination with one or more immune modulators that deplete Tregs and/or inhibit or block the activation of Tregs.

The tryptophan (TRP) to kynurenine (KYN) metabolic pathway is established as a key regulator of innate and adaptive immunity Both the degradation of the essential amino acid tryptophan via indoleamine-2,3-dioxygenase 1 (IDO1)and TRP-2,3-dioxygenase 2 (TDO) and the resulting production of aryl hydrocarbon receptor (AHR) activating tryptophan metabolites, such as kynurenine, is a central pathway maintaining the immunosuppressive microenvironment in many types of cancers. For example, binding of kynurenine to AHR results in reprograming the differentiation of naïve CD4+ T-helper (Th) cells favoring a regulatory T cells phenotype (Treg) while suppressing the differentiation into interleukin-17 (IL-17)-producing Th (Th17) cells. Activation of the aryl hydrogen receptor also results in promoting a tolerogenic phenotype on dendritic cells.

In some embodiments, the compositions and methods of the present disclosure are capable of depleting Tregs or inhibiting or blocking the activation of Tregs by producing tryptophan and/or degrading kynurenine. In some embodiments, the compositions and methods disclosed herein are capable of increasing the CD8+: Treg ratio (e.g., favors the production of CD8+ over Tregs) by producing tryptophan and/or degrading kynurenine.

Thus, in some embodiments, the immune modulator is tryptophan. In other embodiments, the immune modulator is a kynureninase.

In one embodiment, the kynureninase has at least about 80% identity with one or more of SEQ ID NO: 65 through SEQ ID NO: 67. In one embodiment, the kynureninase has at least about 85% identity with one or more of SEQ ID NO: 65 through SEQ ID NO: 67. In one embodiment, the kynureninase has at least about 90% identity with one or more of SEQ ID NO: 65 through SEQ ID NO: 67. In one embodiment, the kynureninase has at least about 95% identity with one or more of SEQ ID NO: 65 through SEQ ID NO: 67. In one embodiment, the kynureninase has at least about 96%, 97%, 98%, or 99% identity with one or more of SEQ ID NO: 65 through SEQ ID NO: 67. Accordingly, in one embodiment, the kynureninase has at least about 80%, 81%, 82%, 83%, 84%, 85%, 86%, 87%, 88%, 89%, 90%, 91%, 92%, 93%, 94%, 95%, 96%, 97%, 98%, or 99% identity with one or more of SEQ ID NO: 65 through SEQ ID NO: 67. In another embodiment, the kynureninase comprises the sequence of one or more of SEQ ID NO: 65 through SEQ ID NO: 67. In another embodiment, the kynureninase consists of the sequence of one or more of SEQ ID NO: 65 through SEQ ID NO: 67.

### Purinergic System—ATP/Adenosine Metabolism

An important barrier to successful cancer immunotherapy is that tumors employ a number of mechanisms to facilitate immune escape, including the production of anti-inflammatory cytokines, the recruitment of regulatory immune subsets, and the production of immunosuppressive metabolites. One such immunosuppressive pathway is the production of extracellular adenosine, a potent immunosuppressive molecule, by CD73. Immune-stimulatory extracellular ATP, released by damaged or dying cells and bacteria, promotes the recruitment of immune phagocytes and activates P2X7R, a coactivator of the NLRP3 inflammasome, which then triggers the production of proinflammatory cytokines, such as IL-1β and IL-18. The catabolism of extracellular ATP into ADP, AMP and adenosine is controlled by CD39 (ecto-nucleoside triphosphate diphosphohydrolase 1, E-NTPDasel) which hydrolyzes ATP into AMP, and CD73 (ecto-5′-nucleotidase, Ecto5′NTase) which dephosphorylates AMP into adenosine by. Thus, CD39 and CD73 act in concert to convert proinflammatory ATP into immunosuppressive adenosine. Beside its immunoregulatory roles, the ectonucleotidase pathway contributes directly to the modulation of cancer cell growth, differentiation, invasion, migration, metastasis, and tumor angiogenesis.

In some embodiments, the compositions and methods disclosed herein comprise a means for removing excess adenosine from the tumor microenvironment. Many bacteria scavenge low concentrations of nucleosides from the environment for synthesis of nucleotides and deoxynucleotides by salvage pathways of synthesis. Additionally, in Escherichia coli, nucleosides can be used as the sole source of nitrogen and carbon for growth (Neuhard J, Nygaard P. Biosynthesis and conversion of nucleotides, purines and pyrimidines. In: Neidhardt F C, Ingraham J L, Low K B, Magasanik B, Schaechter M, Umbarger HE, editors. Escherichia coli and Salmonella typhimurium: Cellular and molecular biology. Washington DC: ASM Press; 1987. pp. 445-473).

In some embodiments, the compositions and methods disclosed herein comprise a means for metabolizing or degrading adenosine. Exemplary enzymes useful for adenosine degradation include SEQ ID NO: 71-77.

### Arginine/Arginase I Metabolism

L-Arginine (L-Arg) is a nonessential amino acid that plays a central role in several biological systems including the immune response. L-Arginine is metabolized by arginase I, arginase II, and the inducible nitric oxide synthase. Arginase 1 hydrolyzes L-Arginine into urea and L-ornithine, the latter being the main substrate for the production of polyamines that are required for rapid cell cycle progression in malignancies. A distinct subpopulation of tumor-infiltrating myeloid-derived suppressor cells (MDSC), and not tumor cells themselves, have been shown to produce high levels of arginase I and cationic amino acid transporter 2B, which allow them to rapidly incorporate L-Arginine (L-Arg) and deplete extracellular L-Arg the tumor microenvironment. These cells are potent inhibitors of T-cell receptor expression and antigen-specific T-cell responses and potent inducers of regulatory T cells. Moreover, recent studies by Lanzavecchia have shown that activated T cells also heavily consume L-arginine and rapidly convert it into downstream metabolites, which lead to a marked decrease in intracellular arginine levels after activation. In these studies, addition of exogenous L-arginine to T cell culture medium increased intracellular levels of free L-arginine in T cells, and moreover increased L-arginine levels caused pleiotropic effects on T cell activation, differentiation, and function, ranging from increased bioenergetics and survival to in vivo anti-tumor activity (Geiger et al., (2016) L-Arginine Modulates T Cell Metabolism and Enhances Survival and Anti-tumor Activity; Cell 167,829-842, the contents of which is herein incorporated by reference in its entirety). Accordingly, arginine uptake by T cells, may lead to enhanced and more sustained T cell activation.

Accordingly, in some embodiments, the immune modulator used in the compositions and methods disclosed herein is arginine.

### Th1/CD8-Attracting Chemokines

Chemokines are critical for attracting and recruiting immune cells, e.g., those that activate immune response and those that induce cancer cell apoptosis. Target cells of chemokines express corresponding receptors to which chemokines bind and mediate function. Therefore, the receptors of CC and CXC chemokine are referred to as CCRs and CXCRs, respectively. CC chemokines bind to CC chemokine receptors, and CXC chemokines bind to CXC chemokine receptors. Most receptors usually bind to more than one chemokine, and most chemokines usually bind to more than one receptor.

The chemokine interferon-γ inducible protein 10 kDa (CXCL10) is a member of the CXC chemokine family which binds to the CXCR3 receptor to exert its biological effects. CXCL10 is involved in chemotaxis, induction of apoptosis, regulation of cell growth and mediation of angiostatic effects. CXCL10 is associated with a variety of human diseases including infectious diseases, chronic inflammation, immune dysfunction, tumor development, metastasis and dissemination. More importantly, CXCL10 has been identified as a major biological marker mediating disease severity and may be utilized as a prognostic indicator for various diseases. In this review, we focus on current research elucidating the emerging role of CXCL10 in the pathogenesis of cancer. Understanding the role of CXCL10 in disease initiation and progression may provide the basis for developing CXCL10 as a potential biomarker and therapeutic target for related human malignancies.

CXCL10 and CXCL9 each specifically activate a receptor, CXCR3, which is a seven trans-membrane-spanning G protein-coupled receptor predominantly expressed on activated T lymphocytes (Th1), natural killer (NK) cells, inflammatory dendritic cells, macrophages and B cells. The interferon-induced angiostatic CXC chemokines and interferon-inducible T-cell chemoattractant (I-TAC/CXCL11), also activate CXCR3. These CXC chemokines are preferentially expressed on Th1 lymphocytes.

Immune-mediated, tissue-specific destruction has been associated with Th1 polarization, related chemokines (CXCR3 and CCR5 ligands, such as CXCL10 and CXCL9), and genes associated with the activation of cytotoxic mechanisms. Other studies have shown that long disease-free survival and overall survival in cancers such as early-stage breast cancer, colorectal, lung, hepatocellular, ovarian, and melanoma are consistently associated with the activation of T helper type 1 (Th1) cell-related factors, such as IFN-gamma, signal transducers and activator of transcription 1 (STA1), IL-12, IFN-regulatory factor 1, transcription factor T-bet, immune effector or cytotoxic factors (granzymes), perforin, and granulysin, CXCR3 and CCR6 ligand chemokines (CXCL9, CXCL10, and CCL5), other chemokines (CXCL1 and CCL2), and adhesion molecules (MADCAM1, ICAM1, VCAM1). Chemoattraction and adhesion has been shown to play a critical role in determining the density of intratumoral immune cells. Other studies have shown that up-regulation of CXCL9, CXCL10, and CXCL11 is predictive of treatment responsiveness (particular responsive to adoptive-transfer therapy). Still other studies have shown that chemokines that drive tumor infiltration by lymphocytes predicts survival of patients with hepatocellular carcinoma.

It is now recognized that cancer progression is regulated by both cancer cell-intrinsic and macroenvironmental factors. It has been demonstrated that the presence of T helper 1 (Th1) and/or cytotoxic T cells correlates with a reduced risk of relapse in several cancers and that a pro-inflammatory tumor microenvironment correlates with prolonged survival in a cohort of patients with hepatocellular carcinoma. CXCL10, CCL5, and CCL2 expression has been shown to correlate with tumor infiltration by Th1, CD8⁺ T cells, and natural killer cells. Data shows that CXCL10, CCL5, and CCL2 are the main chemokines attracting Thl, CD8⁺ T cells, and NK cells into the tumor microenvironment. Also, CXCL10 and TLR3 (induces CXCL 10, CCL5, and CCL2) expression correlates with cancer cell apoptosis.

C—X—C motif chemokine 10 (CXCL10), also known as Interferon gamma-induced protein 10 (IP-10) or small-inducible cytokine B10 is an 8.7 kDa protein that in humans is encoded by the CXCL10 gene. CXCL10 is a small cytokine belonging to the CXC chemokine family which is secreted by several cell types in response to IFN-γ, including monocytes, endothelial cells and fibroblasts. CXCL10 plays several roles, including chemoattraction for monocytes/macrophages, T cells, NK cells, and dendritic cells, promotion of T cell adhesion to endothelial cells, antitumor activity, and inhibition of bone marrow colony formation and angiogenesis. This chemokine elicits its effects by binding to the cell surface chemokine receptor CXCR3.

Under proinflammatory conditions CXCL10 is secreted from a variety of cells, such as leukocytes, activated neutrophils, eosinophils, monocytes, epithelial cells, endothelial cells, stromal cells (fibroblasts) and keratinocytes in response to IFN-γ. This crucial regulator of the interferon response, preferentially attracts activated Th1 lymphocytes to the area of inflammation and its expression is associated with Th1 immune responses. CXCL10 is also a chemoattractant for monocytes, T cells and NK cells. (Chew et al., Gut, 2012, 61:427-438. Still other studies have shown that immune -protective signature genes, such as Thl-type chemokines CXCL10 and CXCL9, may be epigenetically silenced in cancer. (Peng et al., Nature, 2015, doi:10.1038/nature 15520).

Chemokine (C—X—C motif) ligand 9 (CXCL9) is a small cytokine belonging to the CXC chemokine family that is also known as Monokine induced by gamma interferon (MIG). CXCL9 is a T-cell chemoattractant (Th1/CD8-attracting chemokine) which is induced by IFN-γ. It is closely related to two other CXC chemokines, CXCL10 and CXCL11. CXCL9, CXCL10 and CXCL11 all elicit their chemotactic functions by interacting with the chemokine receptor CXCR3.

In some embodiments, the immune modulator is one or more chemokines that are Th1/CD8-attracting chemokines. In some embodiments, the immune modulator is one or more chemokines that are CXCR3 ligand chemokines. In some embodiments, the immune modulator is one or more chemokines that are CCRS ligand chemokines. In some embodiments, the immune modulator is CXCL9. In some embodiments, the immune modulator is CXCL10.

In some embodiments, the CXCL10 polypeptide has at least about 80% identity with a sequence selected from SEQ ID NO: 1205 or SEQ ID NO: 1206. In some embodiments, the CXCL10 polypeptide has about having at least about 90% identity with a sequence selected from SEQ ID NO: 1205 or SEQ ID NO: 1206. In some embodiments, the CXCL10 polypeptide has about having at least about 95% identity with a sequence selected from SEQ ID NO: 1205 or SEQ ID NO: 1206. In some embodiments, the CXCL10 polypeptide has about 80%, 81%, 82%, 83%, 84%, 85%, 86%, 87%, 88%, 89%, 90%, 91%, 92%, 93%, 94%, 95%, 96%, 97%, 98%, or 99% identity to a sequence selected from SEQ ID NO: 1205 or SEQ ID NO: 1206, or a functional fragment thereof. In another embodiment, the CXCL10 polypeptide comprises a sequence selected from SEQ ID NO: 1205 or SEQ ID NO: 1206. In yet another embodiment, the CXCL10 polypeptide consists of a sequence selected from SEQ ID NO: 1205 or SEQ ID NO: 1206.

### Stromal Modulation

The accumulation of extracellular matrix (ECM) components can distort the normal architecture of tumor and stromal tissue, causing an abnormal configuration of blood and lymphatic vessels. One factor that may contribute to the therapeutic resistance of a tumor is the rigidity of the ECM that significantly compresses blood vessels, resulting in reduced perfusion (due to constraints on diffusion and convection) that ultimately impedes the delivery of therapeutics to tumor cells. One strategy to reduce vessel compression in the stroma and assist in drug delivery is to enzymatically break down the ECM scaffold, which in some stromal tumor environments consist of fibroblasts, immune cells, and endothelial cells imbedded within a dense and complex ECM with abundant Hyaluronan or Hyaluronic acid (HA). HA is a large linear glycosaminoglycan (GAG) composed of repeating N-acetyl glucosamine and glucuronic acid units that retains water due to its high colloid osmotic pressure. HA is believed to play a role in tumor stroma formation and maintenance. Enzymatic HA degradation by hyaluronidase (PEGPH20; rHuPH20) has been shown to decrease interstitial fluid pressure in mouse pancreatic ductal adenocarcinoma (PDA) tumors with a concomitant observation in vessel patency, drug delivery, and survival (Provenzano et al. Cancer Cell, 2012, 21:418-429; Thompson et al., Mol Cancer Ther, 2010, 9:3052-64). It is believed that PEGPH20 liberates water bound to HA by cleaving the extended polymer into substituent units. The release of trapped water decreases the interstitial fluid pressure to a range of 20-30 mmHg, enabling collapsed arterioles and capillaries to open (Provenzano et al.).

In some embodiments, the immune modulator is a molecule that modulates the stroma. In some embodiments, the immune modulator is an enzyme that degrades Hyaluronan or Hyaluronic acid (HA). In some embodiments, the immune modulator is a hyaluronidase.

In some embodiments, the hyaluronidase polypeptide is selected from SEQ ID NO: 1127, SEQ ID NO: 1128, SEQ ID NO:1129 , SEQ ID NO: 1130, SEQ ID NO: 1131 or functional fragments thereof. In some embodiments, hyaluronidase polypeptide is at least about 80%, at least about 85%, at least about 90%, at least about 95%, or at least about 99% identity to one or more polypeptide(s) selected from selected from SEQ ID NO: 1127, SEQ ID NO: 1128, SEQ ID NO:1129 , SEQ ID NO: 1130, SEQ ID NO: 1131 or a functional fragment thereof.

### Other Immune Modulators

Other immune modulators include therapeutic nucleic acids (RNA and DNA), for example, RNAi molecules (such as siRNA, miRNA, dsRNA), mRNAs, antisense molecules, aptamers, and CRISPER/Cas 9 molecules as described in International Patent Application PCT/US2017/013072, filed Jan. 11, 2017, published as WO2017/123675, the contents of which is herein incorporated by reference in its entirety. Thus, in some embodiments, the immune modulators is an RNA or DNA immune modulator, e.g., including nucleic acid molecules selected from RNAi molecules (siRNA, miRNA, dsRNA), mRNAs, antisense molecules, aptamers, and CRISPR/Cas 9 molecules. Such molecules are exemplified and discussed in the references provided herein below.

### Combinations of Immune Initiators and Immune Sustainers

In some embodiments, the compositions and methods disclosed herein are designed to combine multiple mechanisms. For example, by activating multiple orthogonal immunomodulatory pathways in the tumor microenvironment, immunologically cold tumors are transformed into immunologically hot tumors. Multiple effectors can be selected which have an impact on different components of the immune response. Different immune response components which can be targeted by the bacteria and immune modulators disclosed herein include immune initiation and immune augmentation and T cell expansion (immune sustenance).

In some embodiments, a microorganism and at least a first immune modulator, e.g., an immune initiator or an immune sustainer, may be administered in combination with, e.g., before, at the same time as, or after, at least a second immune modulator, e.g., an immune initiator or an immune sustainer.

Non-limiting examples of immune initiators and sustainers are described in Table 5 and Table 6.

TABLE 5
Immune Initiators

| Effect | Type | Effector |
|---|---|---|
| Immune activation/ | Cytokine/Chemokine | TNFα |
| Oncolysis/Priming | | |
| Immune activation/Priming | Cytokine/Chemokine | IFN-gamma |
| Immune activation/Priming | Cytokine/Chemokine | IFN-beta1 |
| Immune activation/ | Single chain | SIRPα |
| Phagocytosis/Priming | antibodies/Ligands | |
| Immune activation/Priming | Single chain | CD40L |
| | antibodies/Ligands | |
| Immune activation/Priming | Metabolic conversion | STING agonist |
| Immune activation/Priming | Cytokine/Chemokine | GMCSF |
| Immune activation/Priming | T cell co-stimulatory | Agonistic anti- |
| | receptor/Ligands | OX40 antibody or |
| | | agonistic OX40L |
| Immune activation/Priming | T cell co-stimulatory | Agonistic anti- |
| | receptor/Ligands | 41BB antibody or |
| | | agonistic 41BBL |
| Immune activation/Priming | T cell co-stimulatory | Agonistic anti- |
| | receptor/Ligands | GITR antibody or |
| | | agonistic GITRL |
| Immune activation/Priming | Single chain | Anti-PD-1 antibody, |
| | antibodies/Ligands | anti-PD-L1 antibody |
| | | (antagonistic) |
| Immune activation/Priming | Single chain | Anti-CTLA4 |
| | antibodies/Ligands | antibody |
| | | (antagonistic) |
| Oncolysis/Priming | Engineered | 5FC−>5FU |
| | chemotherapy | |
| Oncolyis/Priming | Lytic peptides | Lytic peptides (e.g. |
| | | azurin) |
| Immune activation/Priming | Metabolic conversion | Arginine |

TABLE 6
Immune Sustainers

| Effect | Type | Effector |
|---|---|---|
| Immune Augmentation/ | Single chain | Anti-PD-1antibody, |
| Reversal of Exhaustion | antibodies/Ligands | anti-PD-L1 antibody |
| | | (antagonistic) |
| Immune Augmentation/ | Single chain | Anti-CTLA4 |
| T cell Expansion | antibodies/Ligands | antibody |
| | | (antagonistic) |
| Immune Augmentation/ | Cytokine/Chemokine | IL-15 |
| T cell Expansion | | |
| Immune Augmentation/ | Cytokine/Chemokine | CXCL10 |
| T cell Recruitment | | |
| Immune Augmentation/ | Metabolic conversion | Arginine |
| T cell Expansion | | |
| Immune Augmentation/ | Metabolic conversion | Adenosine consumer |
| T cell Expansion | | |
| Immune Augmentation/ | Metabolic conversion | Kynureninase |
| T cell Expansion | | |
| Immune Augmentation/ | Co-stimulatory | Agonistic anti- |
| T cell Expansion | Ligand/Receptor | OX40 antibody or |
| | | OX40L |
| Immune Augmentation/ | Co-stimulatory | Agonistic anti- |
| T cell Expansion | Ligand/Receptor | 41BB antibody or |
| | | 41BBL |
| Immune Augmentation/ | Co-stimulatory | Agonistic anti- |
| T cell Expansion | Ligand/Receptor | GITR antibody or |
| | | GITRL |
| Immune Augmentation/ | Cytokine/Chemokine | IL-12 |
| T cell Expansion | | |
| Antigen presentation/ | Cytokine/Chemokine | IFN-gamma |
| Tumor cell targeting | | |

In some combination embodiments, one or more effectors of Table 5 can be combined with one or more effectors of Table 6.

Multiple effectors can be selected which have an impact on different components of the immune response. Different immune response components which can be targeted by the effectors disclosed herein include oncolysis, immune activation of APCs, and activation and priming of T cells (“immune initiator”), trafficking and infiltration, immune augmentation, T cell expansion, (“immune sustainer”). In some combination embodiments, an “immune initiator” is combined with an “immune sustainer”. In some embodiments, an immune initiator and/or an immune sustainer may further be combined with a stromal modulator, e.g., hyaluronidase.

In one embodiment, the immune initiator is not the same as the immune sustainer. As one non-limiting example, where the immune initiator is IFN-gamma, the immune sustainer is not IFN-gamma. In one embodiment, the immune initiator is different than the immune sustainer. As one non-limiting example, where the immune initiator is IFN-gamma, the immune sustainer is not IFN-gamma.

Any one or more immune initiator(s) may be combined with any one or more immune sustainer(s) in the cancer immunity cycle. Accordingly, in some embodiments, the one or more immune initiators modulate, e.g., intensify, one or more of steps of the cancer immunity cycle (1) oncolysis, (2) activation of APCs and/or (3) priming and activation of T cells in combination with one or more immune sustainers, which modulate, e.g., boost, one or more of steps (4) T cell trafficking and infiltration, (5) recognition of cancer cells by T cells and/or T cell support and/or (6) the ability to overcome immune suppression. Non-limiting examples of immune initiators which modulate steps (1), (2), an (3) are provided herein. Non-limiting examples of immune sustainers which modulate steps (4), (5), an (6) are provided herein. Accordingly, any of these exemplary immune modulators may part of an immune initiator /immune sustainer combination which is capable of modulating one or more cancer immunity cycle steps as described herein. Accordingly, combinations of immune initiator(s)/immune sustainer(s) can modulate combinations of cancer immunity cycle step, e.g., as follows: step (1), step (2), step (3), step (4), step (5), step (6); step (1), step (2), step (3), step (4), step (5); step (1), step (2), step (3), step (4), step (6); step (1), step (2), step (3), step (5), step (6); step (1), step (2), step (3), step (4); step (1), step (2), step (3), step (5); step (1), step (2), step (3), step (6); step (1), step (2), step (4), step (5), step (6); step (1), step (2), step (4), step (5); step (1), step (2), step (4), step (6); step (1), step (2), step (5), step (6); step (1), step (2), step (4); step (1), step (2), step (5); step (1), step (2), step (6); step (1), step (3), step (4), step (5), step (6); step (1), step (3), step (4), step (5); step (1), step (3), step (4), step (6); step (1), step (3), step (5), step (6); step (1), step (3), step (4); step (1), step (3), step (5); step (1), step (3), step (6); step (2), step (3), step (4), step (5), step (6); step (2), step (3), step (4), step (5); step (2), step (3), step (4), step (6); step (2), step (3), step (5), step (6); step (2), step (3), step (4); step (2), step (3), step (5); step (2), step (3), step (6); step (1), step (4), step (5), step (6); step (1), step (4), step (5); step (1), step (4), step (6); step (1), step (5), step (6); step (1), step (4); step (1), step (5); step (1), step (6); step (2), step (4), step (5), step (6); step (2), step (4), step (5); step (2), step (4), step (6); step (2), step (5), step (6); step (2), step (4); step (2), step (5); step (2), step (6); step (3), step (4), step (5), step (6); step (3), step (4), step (5); step (3), step (4), step (6); step (3), step (5), step (6); step (3), step (4); step (3), step (5); step (3), step (6).

In any of these embodiments and all combination embodiments, the compositions and methods disclosed herein can be used in conjunction with conventional cancer therapies, such as surgery, chemotherapy, targeted therapies, radiation therapy, tomotherapy, immunotherapy, cancer vaccines, hormone therapy, hyperthermia, stem cell transplant (peripheral blood, bone marrow, and cord blood transplants), photodynamic therapy, oncolytic virus therapy, and blood product donation and transfusion.

### Pharmaceutical Compositions and Formulations

Pharmaceutical compositions comprising the microorganisms and/or immune modulators of the invention may be used to treat, manage, ameliorate, and/or prevent cancer. Pharmaceutical compositions of the invention may be used alone or in combination with prophylactic agents, therapeutic agents, and/or pharmaceutically acceptable carriers are provided.

In some embodiments, the bacteria are administered systemically or intratumorally as spores. As a non-limiting example, the bacteria are Clostridial strains, and administration results in a selective colonization of hypoxic/necrotic areas within the tumor. In some embodiments, the spores germinate exclusively in the hypoxic/necrotic regions present in solid tumors and nowhere else in the body.

The pharmaceutical compositions of the invention may be formulated in a conventional manner using one or more physiologically acceptable carriers comprising excipients and auxiliaries, which facilitate processing of the active ingredients into compositions for pharmaceutical use. Methods of formulating pharmaceutical compositions are known in the art (see, e.g., “Remington's Pharmaceutical Sciences,” Mack Publishing Co., Easton, Pa.). In some embodiments, the pharmaceutical compositions are subjected to tableting, lyophilizing, direct compression, conventional mixing, dissolving, granulating, levigating, emulsifying, encapsulating, entrapping, or spray drying to form tablets, granulates, nanoparticles, nanocapsules, microcapsules, microtablets, pellets, or powders, which may be enterically coated or uncoated. Appropriate formulation depends on the route of administration.

The compositions may be formulated into pharmaceutical compositions in any suitable dosage form (e.g., liquids, capsules, sachet, hard capsules, soft capsules, tablets, enteric coated tablets, suspension powders, granules, or matrix sustained release formations for oral administration) and for any suitable type of administration (e.g., oral, topical, injectable, intravenous, sub-cutaneous, intratumoral, peritumor, immediate-release, pulsatile-release, delayed-release, or sustained release). Suitable dosage amounts for the bacteria may range from about 10⁴ to 10¹² bacteria. The composition may be administered once or more daily, weekly, or monthly. The composition may be administered before, during, or following a meal. In one embodiment, the pharmaceutical composition is administered before the subject eats a meal. In one embodiment, the pharmaceutical composition is administered currently with a meal. In on embodiment, the pharmaceutical composition is administered after the subject eats a meal.

The bacteria and/or immune modulator(s) may be formulated into pharmaceutical compositions comprising one or more pharmaceutically acceptable carriers, thickeners, diluents, buffers, buffering agents, surface active agents, neutral or cationic lipids, lipid complexes, liposomes, penetration enhancers, carrier compounds, and other pharmaceutically acceptable carriers or agents. For example, the pharmaceutical composition may include, but is not limited to, the addition of calcium bicarbonate, sodium bicarbonate, calcium phosphate, various sugars and types of starch, cellulose derivatives, gelatin, vegetable oils, polyethylene glycols, and surfactants, including, for example, polysorbate 20. In some embodiments, the bacteria of the invention may be formulated in a solution of sodium bicarbonate, e.g., 1 molar solution of sodium bicarbonate (to buffer an acidic cellular environment, such as the stomach, for example).

The compositions may be administered intravenously, e.g., by infusion or injection. Alternatively, the compositions may be administered intratumorally and/or peritumorally. In other embodiments, the compositions may be administered intra-arterially, intramuscularly, or intraperitoneally. In some embodiments, the bacteria colonize about 20%, 30%, 40%, 50%, 60%, 70%, 80%, 90% or more of the tumor. In some embodiments, the bacteria and/or immune modulator(s) are co-administered with a PEGylated form of rHuPH20 (PEGPH20) or other agent in order to destroy the tumor septae in order to enhance penetration of the tumor capsule, collagen, and/or stroma.

The microorganisms and/or immune modulator(s) of the disclosure may be administered via intratumoral injection. Intratumoral injection may elicit a potent localized inflammatory response as well as an adaptive immune response against tumor cells. In some embodiments, the tumor is injected with an 18-gauge multipronged needle (Quadra-Fuse, Rex Medical). The injection site is aseptically prepared. If available, ultrasound or CT may be used to identify a necrotic region of the tumor for injection. If a necrotic region is not identified, the injection can be directed to the center of the tumor. The needle is inserted once into a predefined region, and dispensed with even pressure. The injection needle is removed slowly, and the injection site is sterilized.

Direct intratumoral injection of the compositions of the invention into solid tumors may be advantageous as compared to intravenous administration. Using an intravenous injection method, only a small proportion of the bacteria may reach the target tumor. For example, following E. coli Nissle injection into the tail vein of 4T1 tumor-bearing mice, most bacteria (>99%) are quickly cleared from the animals and only a small percentage of the administered bacteria colonize the tumor (Stritzker et al., 2007). In particular, in large animals and human patients, which have relatively large blood volumes and relatively small tumors compared to mice, intratumoral injection may be especially beneficial. Injection directly into the tumor allows the delivery of a higher concentration of therapeutic agent and avoids the toxicity, which can result from systemic administration. In addition, intratumoral injection of bacteria induces robust and localized immune responses within the tumor.

Depending on the location, tumor type, and tumor size, different administration techniques may be used, including but not limited to, cutaneous, subcutaneous, and percutaneous injection, therapeutic endoscopic ultrasonography, or endobronchial intratumor delivery. Prior to the intratumor administration procedures, sedation in combination with a local anesthetic and standard cardiac, pressure, and oxygen monitoring, or full anesthesia of the patient is performed.

For some tumors, percutaneous injection can be employed, which is the least invasive administration method. Ultrasound, computed tomography (CT) or fluoroscopy can be used as guidance to introduce and position the needle. Percutaneous intratumoral injection is for example described for hepatocellular carcinoma in Lencioni et al., 2010. Intratumoral injection of cutaneous, subcutaneous, and nodal tumors is for example described in WO/2014/036412 (Amgen) for late stage melanoma.

Single insertion points or multiple insertion points can be used in percutaneous injection protocols. Using a single insertion point, the solution may be injected percutaneously along multiple tracks, as far as the radial reach of the needle allows. In other embodiments, multiple injection points may be used if the tumor is larger than the radial reach of the needle. The needle can be pulled back without exiting, and redirected as often as necessary until the full dose is injected and dispersed. To maintain sterility, a separate needle is used for each injection. Needle size and length varies depending on the tumor type and size.

In some embodiments, the tumor is injected percutaneously with an 18-gauge multipronged needle (Quadra-Fuse, Rex Medical). The device consists of an 18 gauge puncture needle 20 cm in length. The needle has three retractable prongs, each with four terminal side holes and a connector with extension tubing clamp. The prongs are deployed from the lateral wall of the needle. The needle can be introduced percutaneously into the center of the tumor and can be positioned at the deepest margin of the tumor. The prongs are deployed to the margins of the tumor. The prongs are deployed at maximum length and then are retracted at defined intervals. Optionally, one or more rotation-injection-rotation maneuvers can be performed, in which the prongs are retracted, the needle is rotated by a 60 degrees, which is followed by repeat deployment of the prongs and additional injection.

Therapeutic endoscopic ultrasonography (EUS) is employed to overcome the anatomical constraints inherent in gaining access to certain other tumors (Shirley et al., 2013). EUS-guided fine needle injection (EUS-FNI) has been successfully used for antitumor therapies for the treatment of head and neck, esophageal, pancreatic, hepatic, and adrenal masses (Verna et al, 2008). EUS-FNI has been extensively used for pancreatic cancer injections. Fine-needle injection requires the use of the curvilinear echoendoscope. The esophagus is carefully intubated and the echoendoscope is passed into the stomach and duodenum where the pancreatic examination occurs, and the target tumor is identified. The largest plane is measured to estimate the tumor volume and to calculate the injection volume. The appropriate volume is drawn into a syringe. A primed 22-gauge fine needle aspiration (FNA) needle is passed into the working channel of the echoendoscope. Under ultrasound guidance, the needle is passed into the tumor. Depending on the size of the tumor, administration can be performed by dividing the tumor into sections and then injecting the corresponding fractions of the volume into each section. Use of an installed endoscopic ultrasound processor with Doppler technology assures there are no arterial or venous structures that may interfere with the needle passage into the tumor (Shirley et al., 2013). In some embodiments, ‘multiple injectable needle’ (MIN) for EUS-FNI can be used to improvement the injection distribution to the tumor in comparison with straight-type needles (Ohara et al., 2013).

Intratumoral administration for lung cancer, such as non-small cell lung cancer, can be achieved through endobronchial intratumor delivery methods, as described in Celikoglu et al., 2008. Bronchoscopy (trans-nasal or oral) is conducted to visualize the lesion to be treated. The tumor volume can be estimated visually from visible length-width height measurements over the bronchial surface. The needle device is then introduced through the working channel of the bronchoscope. The needle catheter, which consists of a metallic needle attached to a plastic catheter, is placed within a sheath to prevent damage by the needle to the working channel during advancement. The needle size and length varies and is determined according to tumor type and size of the tumor. Needles made from plastic are less rigid than metal needles and are ideal, since they can be passed around sharper bends in the working channel. The needle is inserted into the lesion and the bacteria of the invention are in injected. Needles are inserted repeatedly at several insertion points until the tumor mass is completely perfused. After each injection, the needle is withdrawn entirely from the tumor and is then embedded at another location. At the end of the bronchoscopic injection session, removal of any necrotic debris caused by the treatment may be removed using mechanical dissection, or other ablation techniques accompanied by irrigation and aspiration.

In some embodiments, the compositions are administrated directly into the tumor using methods, including but not limited to, percutaneous injection, EUS-FNI, or endobronchial intratumor delivery methods. In some cases, other techniques, such as laparoscopic or open surgical techniques are used to access the target tumor, however, these techniques are much more invasive and bring with them much greater morbidity and longer hospital stays.

In some embodiments, bacteria, e.g., E. coli Nissle, or spores, e.g., Clostridium novyi NT, are dissolved in sterile phosphate buffered saline (PBS) for systemic or intratumor injection.

The dose to be injected is derived from the type and size of the tumor. The dose of a drug or the bacteria is typically lower, e.g., orders of magnitude lower, than a dose for systemic intravenous administration.

The volume injected into each lesion is based on the size of the tumor. To obtain the tumor volume, a measurement of the largest plane can be conducted. The estimated tumor volume can then inform the determination of the injection volume as a percentage of the total volume. For example, an injection volume of approximately 20-40% of the total tumor volume can be used.

For example, as is for example described in WO/2014/036412, for tumors larger than 5 cm in their largest dimension, up to 4 ml can be injected. For tumors between 2.5 and 5 cm in their largest dimension, up to 2 nil can be injected. For tumors between 2.5 and 5 cm in their largest dimension, up to 2 ml can be injected. For tumors between 1.5 and 2.5 cm in their largest dimension, up to 1 ml can be injected. For tumors between 0.5 and 1.5 cm in their largest dimension, up to 0.5 nil can be injected. For tumors equal or small than 0.5 in their largest dimension, up to 0.1 ml can be injected. Alternatively, ultrasound scan can be used to determine the injection volume that can be taken up by the tumor without leakage into surrounding tissue.

In some embodiments, the treatment regimen will include one or more intratumoral administrations. In some embodiments, a treatment regimen will include an initial dose, which followed by at least one subsequent dose. One or more doses can be administered sequentially in two or more cycles.

For example, a first close may be administered at day 1, and a second dose may be administered after 1, 2, 3, 4, 5, 6, days or 1, 2, 3, or 4 weeks or after a longer interval. Additional doses may be administered after 1, 2, 3, 4, 5, 6, days or after 1, 2, 3, or 4 weeks or longer intervals. In some embodiments, the first and subsequent administrations have the same dosage. In other embodiments, different doses are administered. In some embodiments, more than one dose is administered per day, for example, two, three or more doses can be administered per day.

The routes of administration and dosages described are intended only as a guide. The optimum route of administration and dosage can be readily determined by a skilled practitioner. The dosage may be determined according to various parameters, especially according to the location of the tumor, the size of the tumor, the age, weight and condition of the patient to be treated and the route and method of administration.

In some embodiments, the bacteria is administered via first route, e.g., intratumoral injection, and the at least one immune modulator is administered via a second route, e.g., orally.

In some embodiments, the compositions of the disclosure may be administered orally. In some embodiments, the compositions may be useful in the prevention, treatment or management of liver cancer or liver metastases. For example, Danino et al showed that orally administered E. coli Nissle is able to colonize liver metastases by crossing the gastrointestinal tract in a mouse model of liver metastases (Danino et al., Programmable probiotics for detection of cancer in urine. Science Translational Medicine, 7 (289): 1-10, the contents of which is herein incorporated by reference in its entirety).

In one embodiment, the composition is delivered by intratumor injection. In one embodiment, the composition is delivered intrapleurally. In one embodiment, the composition is delivered subcutaneously. In one embodiment, the composition is delivered intravenously. In one embodiment, the composition is delivered intrapleurally.

In some embodiments, the compositions may be administered intratumorally according to a regimen which requires multiple injections. In some embodiments, the bacteria and at least one immune modulator are administered together in each intratumoral injection. In some embodiments, a bacteria strain is injected first and an immune modulator is injected at a later timepoint. In other embodiments, an immune modulator is injected first, and a bacteria is injected at a later time point. Additional injections, either concurrently or sequentially, can follow.

Tumor types into which the bacteria of the current invention are intratumorally delivered include locally advanced and metastatic tumors, including but not limited to, B, T, and NK cell lymphomas, colon and rectal cancers, melanoma, including metastatic melanoma, mycosis fungoides, Merkel carcinoma, liver cancer, including hepatocellular carcinoma and liver metastasis secondary to colorectal cancer, pancreatic cancer, breast cancer, follicular lymphoma, prostate cancer, refractory liver cancer, and Merkel cell carcinoma.

In some embodiments, tumor cell lysis occurs as part of the intratumor injection. As result, tumor antigens may exposed eliciting an anti-tumor response. This exposure may work together with the effector expressed by the bacteria to enhance the anti-tumor effect. In some embodiments, tumor cell lysis does not occur as part of the intratumor injection.

Dosage regimens may be adjusted to provide a therapeutic response. Dosing can depend on several factors, including severity and responsiveness of the disease, route of administration, time course of treatment (days to months to years), and time to amelioration of the disease. For example, a single bolus may be administered at one time, several divided doses may be administered over a predetermined period of time, or the dose may be reduced or increased as indicated by the therapeutic situation. The specification for the dosage is dictated by the unique characteristics of the active compound and the particular therapeutic effect to be achieved. Dosage values may vary with the type and severity of the condition to be alleviated. For any particular subject, specific dosage regimens may be adjusted over time according to the individual need and the professional judgment of the treating clinician. Toxicity and therapeutic efficacy of compounds provided herein can be determined by standard pharmaceutical procedures in cell culture or animal models. For example, LD₅₀, ED₅₀, EC₅₀, and IC₅₀ may be determined, and the dose ratio between toxic and therapeutic effects (LD₅₀/ED₅₀) may be calculated as the therapeutic index. Compositions that exhibit toxic side effects may be used, with careful modifications to minimize potential damage to reduce side effects. Dosing may be estimated initially from cell culture assays and animal models. The data obtained from in vitro and in vivo assays and animal studies can be used in formulating a range of dosage for use in humans.

The ingredients are supplied either separately or mixed together in unit dosage form, for example, as a dry lyophilized powder or water-free concentrate in a hermetically sealed container such as an ampoule or sachet indicating the quantity of active agent. If the mode of administration is by injection, an ampoule of sterile water for injection or saline can be provided so that the ingredients may be mixed prior to administration.

The pharmaceutical compositions may be packaged in a hermetically sealed container such as an ampoule or sachet indicating the quantity of the agent. In one embodiment, one or more of the pharmaceutical compositions is supplied as a dry sterilized lyophilized powder or water-free concentrate in a hermetically sealed container and can be reconstituted (e.g., with water or saline) to the appropriate concentration for administration to a subject. In an embodiment, one or more of the prophylactic or therapeutic agents or pharmaceutical compositions is supplied as a dry sterile lyophilized powder in a hermetically sealed container stored between 2° C. and 8° C. and administered within 1 hour, within 3 hours, within 5 hours, within 6 hours, within 12 hours, within 24 hours, within 48 hours, within 72 hours, or within one week after being reconstituted. Cryoprotectants can be included for a lyophilized dosage form, principally 0-10% sucrose (optimally 0.5-1.0%). Other suitable cryoprotectants include trehalose and lactose. Other suitable bulking agents include glycine and arginine, either of which can be included at a concentration of 0-0.05%, and polysorbate-80 (optimally included at a concentration of 0.005-0.01%). Additional surfactants include but are not limited to polysorbate 20 and BRIJ surfactants. The pharmaceutical composition may be prepared as an injectable solution and can further comprise an agent useful as an adjuvant, such as those used to increase absorption or dispersion, e.g., hyaluronidase.

In some embodiments, the composition is formulated for intravenous administration, intratumor administration, or peritumor administration. The composition may be formulated as depot preparations. Such long acting formulations may be administered by implantation or by injection. For example, the compositions may be formulated with suitable polymeric or hydrophobic materials (e.g., as an emulsion in an acceptable oil) or ion exchange resins, or as sparingly soluble derivatives (e.g., as a sparingly soluble salt).

### Methods of Treatment

Another aspect of the invention provides methods of treating cancer. In some embodiments, the invention provides methods for reducing, ameliorating, or eliminating one or more symptom(s) associated with cancer. In some embodiments, the cancer is selected from adrenal cancer, adrenocortical carcinoma, anal cancer, appendix cancer, bile duct cancer, bladder cancer, bone cancer (e.g., Ewing sarcoma tumors, osteosarcoma, malignant fibrous histiocytoma), brain cancer (e.g., astrocytomas, brain stem glioma, craniopharyngioma, ependymoma), bronchial tumors, central nervous system tumors, breast cancer, Castleman disease, cervical cancer, colon cancer, rectal cancer, colorectal cancer, endometrial cancer, esophageal cancer, eye cancer, gallbladder cancer, gastrointestinal cancer, gastrointestinal carcinoid tumors, gastrointestinal stromal tumors, gestational trophoblastic disease, heart cancer, Kaposi sarcoma, kidney cancer, laryngeal cancer, hypopharyngeal cancer, leukemia (e.g., acute lymphoblastic leukemia, acute myeloid leukemia, chronic lymphocytic leukemia, chronic myelogenous leukemia), liver cancer, lung cancer, lymphoma (e.g., AIDS-related lymphoma, Burkitt lymphoma, cutaneous T cell lymphoma, Hogkin lymphoma, Non-Hogkin lymphoma, primary central nervous system lymphoma), malignant mesothelioma, multiple myeloma, myelodysplastic syndrome, nasal cavity cancer, paranasal sinus cancer, nasopharyngeal cancer, neuroblastoma, oral cavity cancer, oropharyngeal cancer, osteosarcoma, ovarian cancer, pancreatic cancer, penile cancer, pituitary tumors, prostate cancer, retinoblastoma, rhabdomyosarcoma, rhabdoid tumor, salivary gland cancer, sarcoma, skin cancer (e.g., basal cell carcinoma, melanoma), small intestine cancer, stomach cancer, teratoid tumor, testicular cancer, throat cancer, thymus cancer, thyroid cancer, unusual childhood cancers, urethral cancer, uterine cancer, uterine sarcoma, vaginal cancer, vulvar cancer, Waldenström macroglobulinemia, and Wilms tumor. In some embodiments, the symptom(s) associated thereof include, but are not limited to, anemia, loss of appetite, irritation of bladder lining, bleeding and bruising (thrombocytopenia), changes in taste or smell, constipation, diarrhea, dry mouth, dysphagia, edema, fatigue, hair loss (alopecia), infection, infertility, lymphedema, mouth sores, nausea, pain, peripheral neuropathy, tooth decay, urinary tract infections, and/or problems with memory and concentration.

The method may comprise preparing a pharmaceutical composition with at least one species, strain, or subtype of bacteria and/or immune modulator described herein, and administering the pharmaceutical composition to a subject in a therapeutically effective amount. The composition may be administered locally, e.g., intratumorally or peritumorally into a tissue or supplying vessel, or systemically, e.g., intravenously by infusion or injection. In some embodiments, the compositions are administered intravenously, intratumorally, intra-arterially, intramuscularly, intraperitoneally, orally, or topically. In some embodiments, the compositions are administered intravenously, i.e., systemically.

In certain embodiments, administering the pharmaceutical composition to the subject reduces cell proliferation, tumor growth, and/or tumor volume in a subject. In some embodiments, the methods of the present disclosure may reduce cell proliferation, tumor growth, and/or tumor volume by at least about 10% to 20%, 20% to 25%, 25% to 30%, 30% to 40%, 40% to 50%, 50% to 60%, 60% to 70%, 70% to 75%, 75% to 80%, 80% to 85%, 85% to 90%, 90% to 95%, 95% to 99%, or more as compared to levels in an untreated or control subject. In some embodiments, reduction is measured by comparing cell proliferation, tumor growth, and/or tumor volume in a subject before and after administration of the pharmaceutical composition. In some embodiments, the method of treating or ameliorating a cancer in a subject allows one or more symptoms of the cancer to improve by at least about 10%, 20%, 30%, 40%, 50%, 60%, 70%, 80%, 90%, 95%, or more.

Before, during, and after the administration of the pharmaceutical composition, cancerous cells and/or biomarkers in a subject may be measured in a biological sample, such as blood, serum, plasma, urine, peritoneal fluid, and/or a biopsy from a tissue or organ. In some embodiments, the methods may include administration of the compositions of the invention to reduce tumor volume in a subject to an undetectable size, or to less than about 1%, 2%, 5%, 10%, 20%, 25%, 30%, 40%, 50%, 60%, 70%, 75%, 80%, or 90% of the subject's tumor volume prior to treatment. In other embodiments, the methods may include administration of the compositions of the invention to reduce the cell proliferation rate or tumor growth rate in a subject to an undetectable rate, or to less than about 1%, 2%, 5%, 10%, 20%, 25%, 30%, 40%, 50%, 60%, 70%, 75%, 80%, or 90% of the rate prior to treatment.

Response patterns may be different than for traditional cytotoxic therapies. For example, tumors treated with immune-based therapies may enlarge before they regress, and/or new lesions may appear (Agarwala et al., 2015). Increased tumor size may be due to heavy infiltration with lymphocytes and macrophages that are normally not present in tumor tissue. Additionally, response times may be slower than response times associated with standard therapies, e.g., cytotoxic therapies. In some embodiments, delivery of the immune modulator may modulate the growth of a subject's tumor and/or ameliorate the symptoms of a cancer while temporarily increasing the volume and/or size of the tumor.

The may be destroyed, e.g., by defense factors in tissues or blood serum (Sonnenborn et al., 2009), or by activation of a kill switch, several hours or days after administration. Thus, the pharmaceutical composition may be re-administered at a therapeutically effective dose and frequency. In alternate embodiments, the bacteria are not destroyed within hours or days after administration and may propagate in the tumor and colonize the tumor.

The pharmaceutical composition may be administered alone or in combination with one or more additional therapeutic agents, e.g., a chemotherapeutic drug or a checkpoint inhibitor, e.g., as described herein and known in the art. An important consideration in selecting the one or more additional therapeutic agents is that the agent(s) should be compatible with the bacteria of the invention, e.g., the agent(s) must not kill the bacteria. In some studies, the efficacy of anticancer immunotherapy, e.g., CTLA-4 or PD-1 inhibitors, requires the presence of particular bacterial strains in the microbiome (Ilda et al., 2013; Vetizou et al., 2015; Sivan et al., 2015). In some embodiments, the pharmaceutical composition comprising the bacteria augments the effect of a checkpoint inhibitor or a chemotherapeutic agent, e.g., allowing lowering of a the dose of systemically administrated chemotherapeutic or immunotherapeutic agents. In some embodiments, the pharmaceutical composition is administered with one or more commensal or probiotic bacteria, e.g., Bifidobacterium or Bacteroides.

In certain embodiments, the pharmaceutical composition may be administered to a subject for treating cancer by administering a bacterium to the subject, and administering at least one immune modulator to the subject. In some embodiments, the administering steps are performed at the same time. In some embodiments, administering the bacterium to the subject occurs before the administering of the at least one immune modulator to the subject. In some embodiments, administering of the at least one immune modulator to the subject occurs before the administering of the bacterium to the subject.

### Chemotherapeutic Agents

In some embodiments, the pharmaceutical compositions are administered sequentially, simultaneously, or subsequently to dosing with one or more chemotherapeutic agents. In some embodiments, the pharmaceutical compositions are administered sequentially, simultaneously, or subsequently to dosing with one or more chemotherapeutic agents selected from Trabectedin®, Belotecan®, Cisplatin®, Carboplatin ®, Bevacizumab®, Pazopanib®, 5-Fluorouracil, Capecitabine®, Irinotecan®, and Oxaliplatin®. In some embodiments, the pharmaceutical compositions are administered sequentially, simultaneously, or subsequently to dosing with gemcitabine (Gemzar). In some embodiments, the pharmaceutical compositions are administered sequentially, simultaneously, or subsequently to dosing with cyclophosphamide. In any of these embodiments, the one or more bacteria are administered systemically or orally or intratumorally.

In some embodiments, one or more pharmaceutical compositions are administered sequentially, simultaneously, or subsequently to dosing with one or more chemotherapeutic agents. In some embodiments, the chemotherapeutic agent is administered systemically, and the bacteria are administered intratumorally. In some embodiments, the chemotherapeutic agent and pharmaceutical composition are administered systemically. In one embodiment, the chemotherapeutic agent is cyclophosphamide.

In some embodiments, the pharmaceutical compositions are able to improve anti-tumor activity (e.g., tumor proliferation, size, volume, weight) of the co-administered chemotherapeutic agent (e.g., cyclophosphamide or another agent described herein or known in the art), e.g., by 10% to 20%, 20% to 25%, 25% to 30%, 30% to 40%, 40% to 50%, 50% to 60%, 60% to 70%, 70% to 75%, 75% to 80%, 80% to 85%, 85% to 90%, 90% to 95%, 95% to 99%, or more as compared to a chemotherapy alone under the same conditions. In some embodiments, the pharmaceutical compositions are able to improve anti-tumor activity (e.g., tumor proliferation, size, volume, weight) of the co-administered chemotherapeutic agent (e.g., cyclophosphamide or another agent described herein or known in the art), e.g., 1.0-1.2-fold, 1.2-1.4-fold, 1.4-1.6-fold, 1.6-1.8-fold, 1.8-2-fold, or two-fold more or more as compared to a chemotherapy alone.

### Checkpoint Inhibition

In certain embodiments, the pharmaceutical compositions are administered sequentially, simultaneously, or subsequently to dosing with one or more checkpoint inhibitors, immune stimulatory antibodies (inhibitory or agonistic) or other agonists known in the art or described herein. In certain embodiments, the pharmaceutical compositions are administered sequentially, simultaneously, or subsequently to dosing with one checkpoint inhibitors, immune stimulatory antibodies (inhibitory or agonistic) or other agonists known in the art or described herein. In certain embodiments, the pharmaceutical compositions are administered sequentially, simultaneously, or subsequently to dosing with two checkpoint inhibitors, immune stimulatory antibodies (inhibitory or agonistic) or other agonists known in the art or described herein.

Non-limiting examples of immune checkpoint inhibitors include CTLA-4 antibodies (including but not limited to Ipilimumab and Tremelimumab (CP675206)), anti-4-IBB (CD137, TNFRSF9) antibodies (including but not limited to PF-05082566, and Urelumab), anti CD134 (OX40) antibodies, including but not limited to Anti-OX40 antibody (Providence Health and Services), anti-PD-1 antibodies (including but not limited to Nivolumab, Pidilizumab, Pembrolizumab (MK-3475/SCH900475, lambrolizumab, REGN2810, PD-1 (Agenus)), anti-PD-L1 antibodies (including but not limited to durvalumab (MEDI4736), avelumab (MSB0010718C), and atezolizumab (MPDL3280A, RG7446, RO5541267)), and anti-KIR antibodies (including but not limited to Lirilumab), LAG3 antibodies (including but not limited to BMS-986016), anti-CCR4 antibodies (including but not limited to Mogamulizumab), anti-CD27 antibodies (including but not limited to Varlilumab), anti-CXCR4 antibodies (including but not limited to Ulocuplumab). In some embodiments, the at least one bacterial cell is administered sequentially, simultaneously, or subsequently to dosing with an anti-phosphatidyl serine antibody (including but not limited to Bavituxumab).

In some embodiments, the pharmaceutical compositions are administered sequentially, simultaneously, or subsequently to dosing with one or more antibodies selected from TLR9 antibody (including, but not limited to, MGN1703 PD-1 antibody (including, but not limited to, SHR-1210 (Incyte/Jiangsu Hengrui)), anti-OX40 antibody (including, but not limited to, OX40 (Agenus)), anti-Tim3 antibody (including, but not limited to, Anti-Tim3 (Agenus/INcyte)), anti-Lag3 antibody (including, but not limited to, Anti-Lag3 (Agenus/INcyte)), anti-B7H3 antibody (including, but not limited to, Enoblituzumab (MGA-271), anti-CT-011 (hBAT, hBAT1) as described in WO2009101611, anti-PDL-2 antibody (including, but not limited to, AMP-224 (described in WO2010027827 and WO2011066342)), anti-CD40 antibody (including, but not limited to, CP-870, 893), anti-CD40 antibody (including, but not limited to, CP-870, 893).

### Co-Stimulatory Molecules

In certain embodiments, one or more bacteria and/or immune modulators are administered sequentially, simultaneously, or subsequently to dosing with one or more agonistic immune stimulatory molecules or agonists, including but not limited to, agonistic antibodies.

In some embodiments, the one or more antibodies are selected from anti-OX40 antibody (including, but not limited to, INCAGN01949 (Agenus); BMS 986178 (Bristol-Myers Squibb), MEDI0562 (Medimmune), GSK3174998 (GSK), PF-04518600 (Pfizer)), anti-41BB/CD137 (including but not limited to PF-05082566 (Pfizer), urelumab (BMS-663513; Bristol-Myers Squibb), arid anti-GITR (including but not limited to TRX518 (Leap Therapeutics), MK-4166 (Merck), MK-1248 (Merck), AMG 228 (Amgen), BMS-986156 (BMS), INCAGN01876 (Incyte/Agenus), MEDI1873 (AZ), GWN323 (NVS).

In some embodiments, the microorganisms and/or immune modulators may be administered as part of a regimen, which includes other treatment modalities or combinations of other modalities. Non-limiting examples of these modalities or agents are conventional therapies (e.g., radiotherapy, chemotherapy), other immunotherapies, stem cell therapies, and targeted therapies, (e.g., BRAF or vascular endothelial growth factor inhibitors; antibodies or compounds), bacteria described herein, and oncolytic viruses. Therapies also include related to antibody-immune engagement, including Fc-mediated ADCC therapies, therapies using bispecific soluble scFvs linking cytotoxic T cells to tumor cells (e.g., BiTE), and soluble TCRs with effector functions. Immunotherapies include vaccines (e.g., viral antigen, tumor associated antigen, neoantigen, or combinations thereof), checkpoint inhibitors, cytokine therapies, adoptive cellular therapy (ACT). ACT includes but is not limited to, tumor infiltrating lymphocyte (TIL) therapies, native or engineered TCR or CAR-T therapies, natural killer cell therapies, and dendritic cell vaccines or other vaccines of other antigen presenting cells. Targeted therapies include antibodies and chemical compounds, and include for example antiangiogenic strategies and BRAF inhibition.

The immunostimulatory activity of bacterial DNA is mimicked by synthetic oligodeoxynucleotides (ODNs) expressing unmethylated CpG motifs. Bode et al., Expert Rev Vaccines. 2011 April; 10(4): 499-511. CpG DNA as a vaccine adjuvant. When used as vaccine adjuvants, CpG ODNs improve the function of professional antigen-presenting cells and boost the generation of humoral and cellular vaccine-specific immune responses. In some embodiments, CpG can be administered in combination with the bacteria of the invention.

In one embodiment, the microorganisms are administered in combination with tumor cell lysates.

The dosage of the pharmaceutical composition and the frequency of administration may be selected based on the severity of the symptoms and the progression of the cancer. The appropriate therapeutically effective dose and the frequency of administration can be selected by a treating clinician.

### Treatment In Vivo

The compositions comprising a bacteria and/or at least one immune modulator may be evaluated in vivo, e.g., in an animal model. Any suitable animal model of a disease or condition associated with cancer may be used, e.g., a tumor syngeneic or xenograft mouse models (see, e.g., Yu et al., 2015). The bacteria and/or at least one immune modulator may be administered to the animal systemically or locally, e.g., via oral administration (gavage), intravenous, or subcutaneous injection or via intratumoral injection, and treatment efficacy determined, e.g., by measuring tumor volume.

Non-limiting examples of animal models include mouse models, as described in Dang et al., 2001, Heap et al., 2014 and Danino et al., 2015).

Pre-clinical mouse models determine which immunotherapies and combination immunotherapies will generate the optimal therapeutic index (maximal anti-tumor efficacy and minimal immune related adverse events (irAEs)) in different cancers.

Implantation of cultured cells derived from various human cancer cell types or a patient's tumor mass into mouse tissue sites has been widely used for generations of cancer mouse models (xenograft modeling). In xenograft modeling, human tumors or cell lines are implanted either subcutaneously or orthotopically into immune-compromised host animals (e.g., nude or SCID mice) to avoid graft rejection. Because the original human tumor microenvironment is not recapitulated in such models, the activity of anti-cancer agents that target immune modulators may not be accurately measured in these models, making mouse models with an intact immune system more desirable.

Accordingly, implantation of murine cancer cells in a syngeneic immunocompetent host (allograft) are used to generate mouse models with tumor tissues derived from the same genetic background as a given mouse strain. In syngeneic models, the host immune system is normal, which may more closely represent the real life situation of the tumor's micro-environment. The tumor cells or cancer cell lines are implanted either subcutaneously or orthotopically into the syngeneic immunocompetent host animal (e.g., mouse). Representative murine tumor cell lines, which can be used in syngeneic mouse models for immune checkpoint benchmarking include, but are not limited to the cell lines listed in International Patent Application PCT/US2017/013072, filed Jan. 11, 2017, published as WO2017/123675, the contents of which is herein incorporated by reference in its entirety.

For tumors derived from certain cell lines, ovalbumin can be added to further stimulate the immune response, thereby increasing the response baseline level. Examples of mouse strains that can be used in syngeneic mouse models, depending on the cell line include C57BL/6, FVB/N, Balb/c, C3H, HeJ, C3H/HeJ, NOD/ShiLT, A/J, 129S1/SvlmJ, NOD. Additionally, several further genetically engineered mouse strains have been reported to mimic human tumorigenesis at both molecular and histologic levels. These genetically engineered mouse models also provide excellent tools to the field and additionally, the cancer cell lines derived from the invasive tumors developed in these models are also good resources for cell lines for syngeneic tumor models Examples of genetically engineered strains are provided in in International Patent Application PCT/US2017/013072, filed Jan. 11, 2017, published as WO2017/123675, the contents of which is herein incorporated by reference in its entirety.

Often potential therapeutic molecules which interact with human immune modulators and stimulate human immune system and do not detect their murine counterparts and vice versa. In studying therapeutic molecules , it is necessary to take this in consideration. More recently, “humanized” mouse models have been developed, in which immunodeficient mice are reconstituted with a human immune system, and which have helped overcome issues relating to the differences between the mouse and human immune systems, allowing the in vivo study of human immunity. Severely immunodeficient mice which combine the IL2receptor null and the severe combined immune deficiency mutation (scid) (NOD-scid IL2Rgnull mice) lack mature T cells, B cells, or functional NK cells, and are deficient in cytokine signaling. These mice can be engrafted with human hematopoietic stem cells and peripheral-blood mononuclear cells. CD34+ hematopoietic stem cells (hu-CD34) are injected into the immune deficient mice, resulting in multi-lineage engraftment of human immune cell populations including very good T cell maturation and function for long-term studies. This model has a research span of 12 months with a functional human immune system displaying T-cell dependent inflammatory responses with no donor cell immune reactivity towards the host. Patient derived xenografts can readily be implanted in these models and the effects of immune modulatory agents studied in an in vivo setting more reflective of the human tumor microenvironment (both immune and non-immune cell-based) (Baia et al., 2015). Human cell lines of interest for use in the humanized mouse models include but are not limited to HCT-116 and HT-29 colon cancer cell lines.

A rat F98 glioma model and the utility of spontaneous canine tumors, as described in Roberts et al 2014 , the contents of each of which are herein incorporated by reference in their entireties. Locally invasive tumors generated by implantation of F98 rat glioma cells engineered to express luciferase were intratumorally injected with C. novyi-NT spores, resulting in germination and a rapid fall in luciferase activity. C. novyi-NT germination was demonstrated by the appearance of vegetative forms of the bacterium. In these studies, C. novyi-NT precisely honed to the tumor sparing neighboring cells.

Canine soft tissue sarcomas for example are common in many breeds and have clinical, histopathological, and genetically features similar to those in humans (Roberts et al, 2014; Staedtke et al., 2015), in particular, in terms of genetic alterations and spectrum of mutations. Roberts et al. conducted a study in dogs, in which C. novyi-NT spores were intratumorally injected (1×10⁸ C. novyi-NT spores) into spontaneously occurring solid tumors in one to 4 treatment cycles and followed for 90 days. A potent inflammatory response was observed, indicating that the intratumoral injections mounted an innate immune response.

In some embodiments, the microorganisms of the invention are administered systemically, e.g., orally, subcutaneously, intravenously or intratumorally into any of the models described herein to assess anti-tumor efficacy and any treatment related adverse side effects.

### EXAMPLES

The following examples provide illustrative embodiments of the disclosure. One of ordinary skill in the art will recognize the numerous modifications and variations that may be performed without altering the spirit or scope of the disclosure. Such modifications and variations are encompassed within the scope of the disclosure. The Examples do not in any way limit the disclosure.

The disclosure provides herein a sequence having at least about 80%, at least about 85%, at least about 90%, at least about 95%, or at least about 99% homologous to the sequence any of the SEQ ID NOs described in the Examples, below.

### Example 1

### Tumor Pharmacokinetics for E. coli Nissle

Tumor pharmacokinetics were assayed and determined as described in are described in International Patent Application PCT/US2017/013072, filed Jan. 11, 2017, published as WO2017/123675, the contents of which is herein incorporated by reference in its entirety. Tumor pharmacokinetics of Nissle (1e7 and 1e8 cells/dose) were determined using a CT26 tumor model over 7 days. Bacterial counts in the tumor tissue were similar at both doses. No bacteria were detected in blood at any of the time points.

Tumor pharmacokinetics of streptomycin resistant Nissle and a Nissle DOM mutant (Nissle ΔPAL::CmR) were compared in a CT26 tumor model. Bacterial counts in the tumor tissue were similar in both strains. No bacteria were detected in the blood. These results indicate that both the wild type and the DOM mutant Nissle can survive in the tumor environment.

Cytokine response in vivo to intratumoral administration of streptomycin resistant Nissle was assessed using a CT26 tumor model at either 1e6 (Group1) or 1e7 cells/dose (Group 2). Levels measured in serum and in the tumor over the time course post SYN94 intratumoral administration in the mouse CT-24 model at the indicated doses. Results indicate that a cytokine response is elicited in the tumor at the higher dose but not in the serum. The lower dose does not elicit a substantial cytokine response. Tumoral PK, levels of bacteria in various tissues and cytokine levels in these tissues were assessed post IT dosing (1e7 cells/dose) at 48 hours. As seen in Internationals Patent Application PCT/US2017/013072, incorporated herein by reference, bacteria were predominantly present in the tumor and absent in other tissues tested. TNFα levels measured were similar in all serum, tumor and liver between SYN94, Saline treated and naïve groups. TNF═ levels are negligible relative to TNFα levels measured at 1.5 hours when Nissle is administered at 1e8 via IV. However, even with IV administration, TNFα levels drop off to undetectable levels at 4 hours. Similar low levels of TNFα are detected at a 1e6 IV dose of SYN94.

### Example 2

### Evaluation of Efficacy of Engineered and Non-engineered Nissle Treatment in the Tumor Models

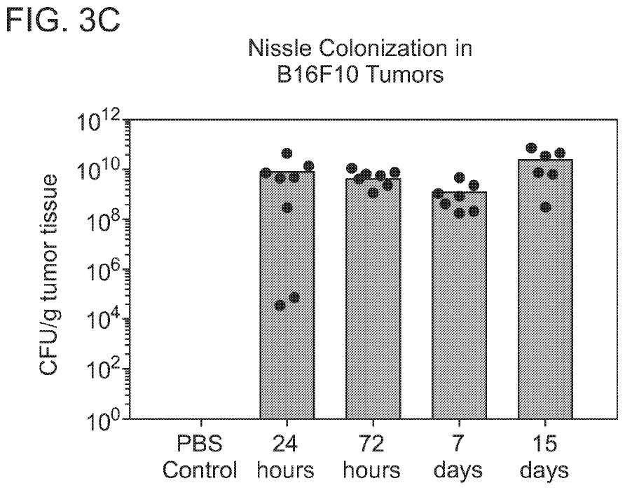

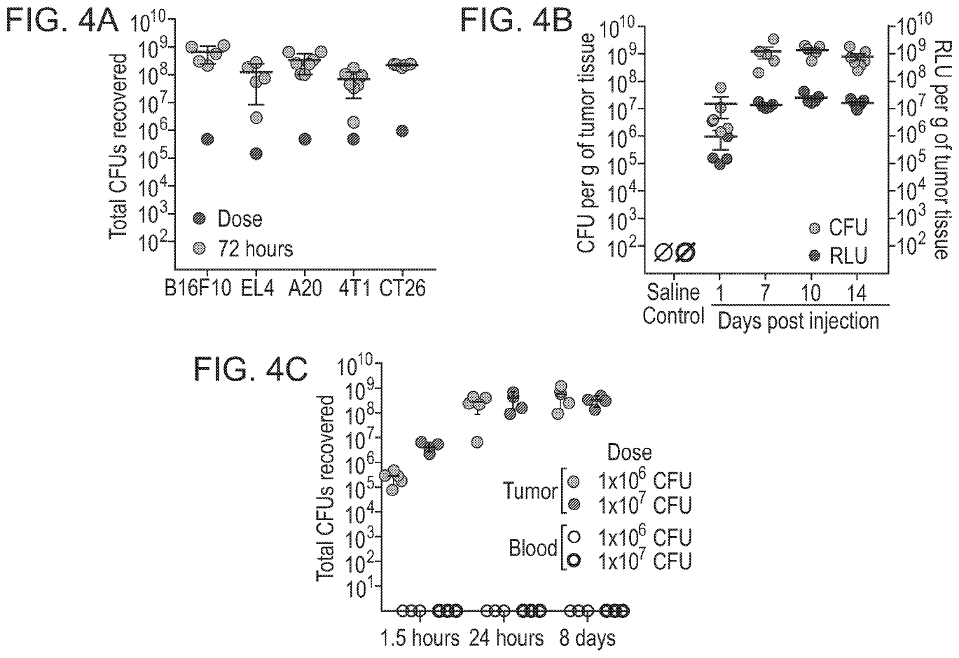

In a first study, intra-tumoral (i.t.) injection of EcN resulted in expansion and colonization in a wide variety of cancer types, including B16.F10, EL4, A20, 4T1 and CT26 transplantable tumors (FIG. 4A). In tumors, EcN rapidly expanded, reaching steady state between ˜24-72 hours, and remained localized to the tumor as bacteria were not detected in serum (FIGS. 4A-4C). To evaluate EcN persistence and metabolic activity, an engineered strain containing the LuxABCDE bioluminescence reporter cassette (EcN-Lux) was used. Following i.t. injection, EcN-Lux expanded in tumors, persisted and exhibited consistent metabolic activity for up to 14 days (measured as bioluminescence) (FIG. 4B). Intratumoral treatment with EcN resulted in dose-dependent increases in IL-6 and TNFα in both tumor and serum at early time points (data not shown), with the magnitude and duration of the response substantially higher within tumors. Finally, i.t. administration of EcN resulted in a significant delay in tumor growth compared to saline injection controls (data not shown). Collectively, these data support the use of EcN as an immunotherapeutic platform which exhibits tumor specific localization and robust intratumoral metabolic activity which would enable production of therapeutic payloads, such as STING agonists.

A second study was undertaken to determine in vivo activity and efficacy of SYNB (comprising a wild-type E. coli Nissle strain containing dual auxotrophies for diaminopimelic acid and thymidine, and deletion of an endogenous phage) and SYNB1891 (comprising the SYNB Nissle strain plus an FNR-inducible dacA from Listeria monocytogenes integrated into the genome to produce the STING agonist ci-di-AMP), over time compared to saline control in the B16.F10 tumor model (murine melanoma).

To produce SYNB and SYNB1891 bacterial cells for the study, frozen vials of SYNB and SYNB1891 were thawed and used to start overnight shake flask cultures to generate enough biomass to inoculate a bioreactor for each culture; the overnight shake flask cultures were incubated for approximately 15 hours at 37° C., 350 RPM. The bioreactors contained 1.5 L of fermentation media containing FM2 fermentation medium (12 g/L soy hydrolysate, 24 g/L yeast extract, 1.7 g/L KH2PO4, 11.4 g/L K2HPO4, 40 g/L glycerol, 0.125 mUL antifoam 204, 10 mM thymidine, and 0.3 g/L diaminopimelic acid) and were inoculated at an OD˜0.1 using the overnight cultures. The bioreactor cultures were grown at 60% DO, 37° C., pH 7.0 until the OD reached 20. To harvest, cells were spun down at 5000 rpm for 30 min, spent media was decanted, cell pellet was resuspended in 15% Glycerol, 100 mM phosphate buffer aliquoted in 2 mL cryovials and frozen at −80 C. Cells were concentration tested by serial plating.

B16.F10 tumor bearing female C57BL/6 mice, 7 weeks of age, were given three injections intratumorally with either SYNB, SYNB1891 or saline. Tumor volumes were measured at various time points until the conclusion of the experiment.

Briefly, B16.F10 cells were implanted (2×10\hat{ }5/mouse/100 μL in PBS) SC into the right flank of each animal on day-8. Tumor growth was monitored until the tumors reached ˜40-100 mm\hat{ }3. On day 1, mice were randomized into groups (N=10 per group) for intratumor dosing as follows: Saline (group 1, vehicle control), SYNB (group 2, 1×10\hat{ }9 CFU) and SYNB1891 (group 3, 1×10\hat{ }9 CFU). Tumor sizes were measured and mice were injected I.T. with bacteria or saline on days 1, 4, and 7.

Animals were dosed with appropriate bacteria based on group or saline (to control for injection) on days 1, 4, and 7. Tumor volumes and body weights were recorded two times in a week with a gap of 1-2 days in between two measurements.

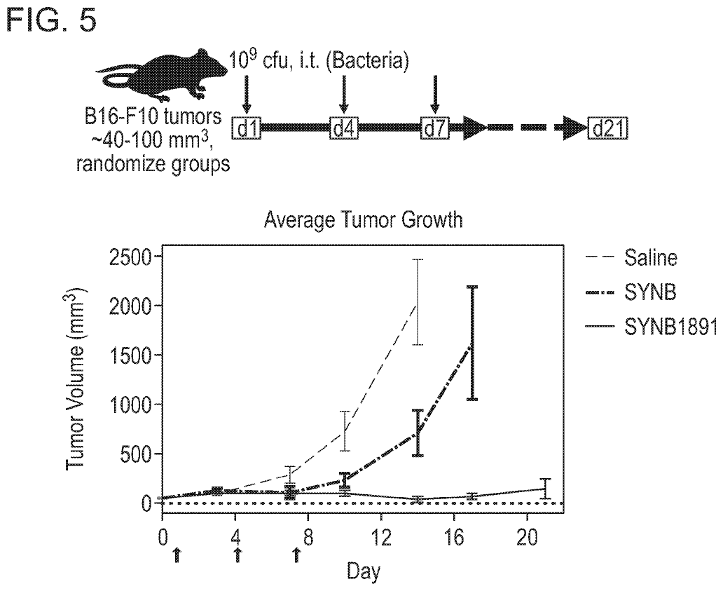

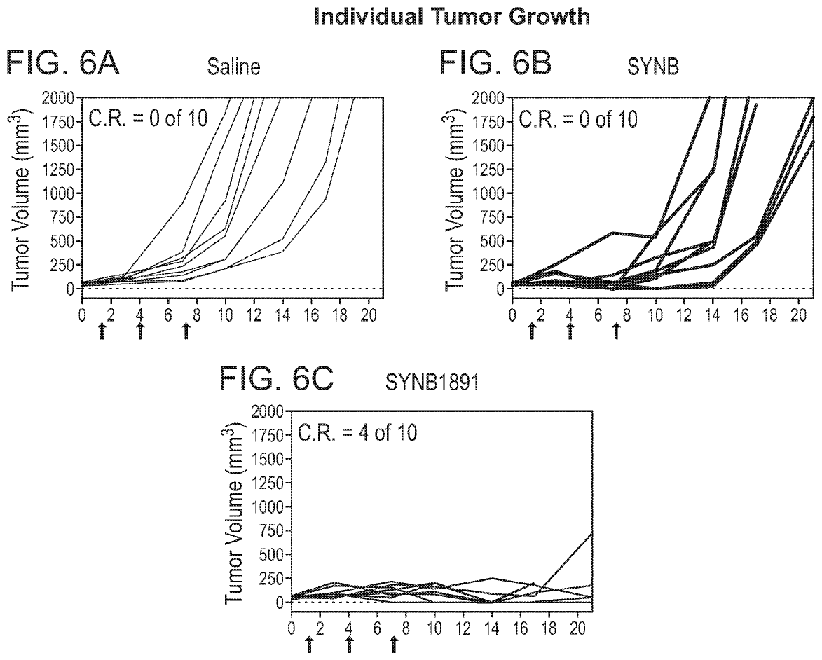

Mean average tumor volumes are shown for each experimental group up to 21 days in FIG. 5, with individual mice shown in FIG. 6A (saline control) , FIG. 6B (SYNB) and FIG. 6C (SYNB1891). Results indicate that administration of the non-engineered SYNB strain alone has a moderate effect and slows tumor growth. Additionally, administration of the STING agonist producing strain, SYNB1891, results significant control of tumor growth with 40% of mice tumor free by day 21.

### Example 3

### Evaluation of Efficacy of STING Agonist Treatment in a Balb/c-A20 Tumor Model

To determine in vivo activity and efficacy of a STING agonist comprising plasmid-based tet-inducible dacA from Listeria monocytogenes, over time at three different doses and compared to PBS control in the c-A20 tumor model (A20 B-cell lymphoma).

To produce cells for the study, overnight cultures were used to inoculate 500 mL LB medium with antibiotic. The strains were incubated with shaking at 37 C until the culture reached the end of log phase (OD600=0.8-1.0). To harvest, cells were spun down at 5000 rpm for 20 min, media was aspirated, cells were washed with PBS, resuspended in 15% Glycerol and PBS, aliquoted and frozen at −80 C. Cells were concentration tested by serial plating.

Female Balb/c mice 6 weeks of age were implanted with A20 tumors, injected intratumorally with three different doses of bacteria producing enzymes capable of producing c-diAMP. Tumor volumes were measured at various time points, while tumors were weighed and processed at the conclusion of the experiment.

Briefly, A-20 cells were implanted (2×10 5/mouse/100 μL in PBS) SC into the right flank of each animal on day-15. Tumor growth was monitored until the tumors reached ˜100 mm\hat{ }3. On day 0, mice were randomized into groups (N=8 per group) for intratumor dosing as follows: PBS (group 1, vehicle control), SYN3527 (group 2, 1×10\hat{ }7 CFU), SYN3527 (group 3, 5×10\hat{ }7 CFU), and SYN3527 (group 4, 5×10\hat{ }8 CFU). Tumor sizes were measured and mice were injected I.T. with bacteria or PBS on day 0, 2, and 5, followed by ATC (lug I.P.) 4 hours later.

Animals were dosed with appropriate bacteria based on group or saline (to control for injection) on days 0, 3, and 7. Four hours after dosing with the bacteria, mice were treated with 10 ug ATC (anhydrotetracycline) via intraperitoneal injection. Tumor volumes and body weights were recorded three times in a week with a gap of 1-2 days in between two measurements.

The resulting tumor volumes would indicate that administration of the strain could drive dose-dependent tumor control in A20 lymphoma model.

### Example 4

### Phagocytosis and STING are Required for SYNB1891-Mediated Type I Interferon Induction

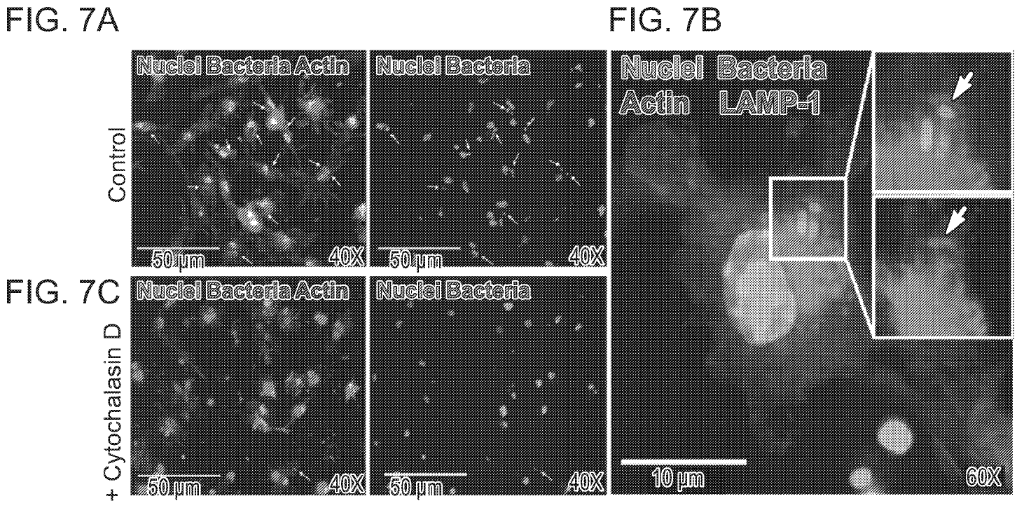

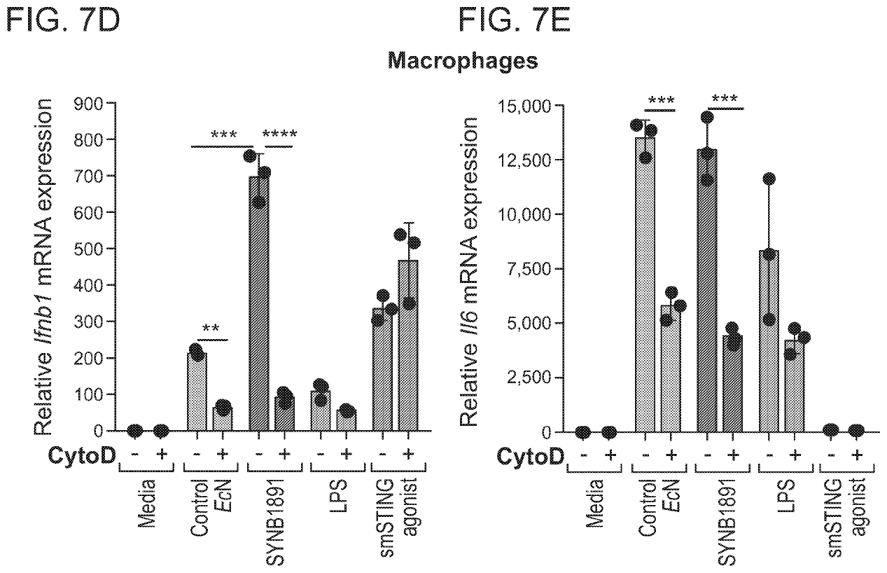

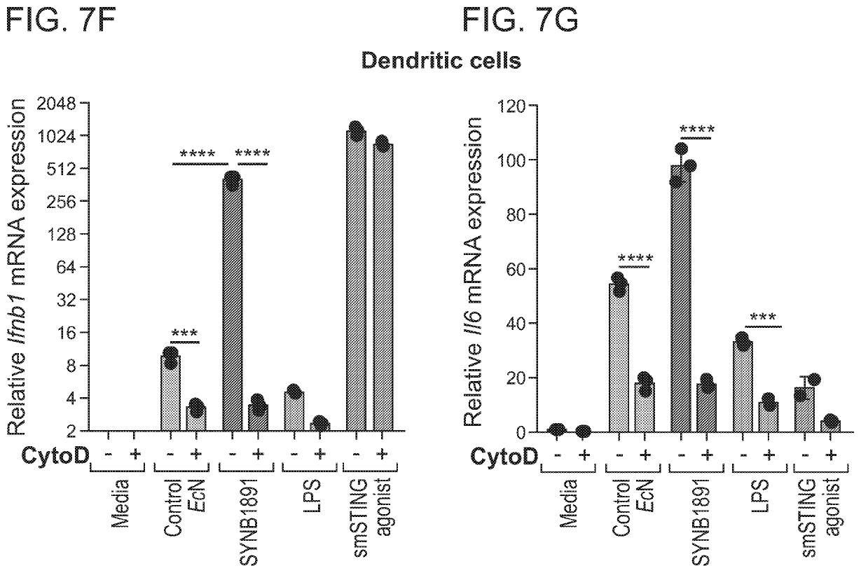

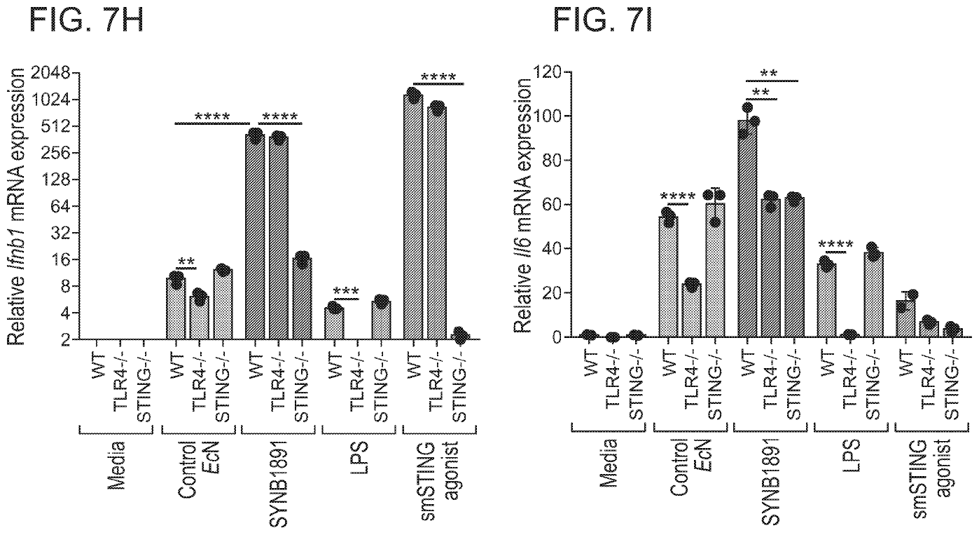

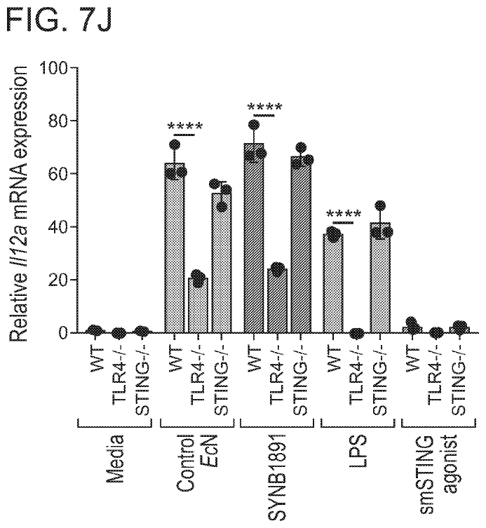

To evaluate the mechanism of action of SYNB1891 in the induction of type I IFNs, bacterial phagocytosis was blocked using cytochalasin D. Cytochalasin D inhibits actin polymerization and prevents phagocytosis, yet it has minimal effect on endocytosis/pinocytosis of soluble small molecules. Co-culture of murine bone marrow-derived dendritic cells (BMDCs) with SYNB1891, modified to express GFP (SYNB1891-gfp), showed many bacterial cells associated with BMDCs and residing within mature phagosomes that contained lysosome-associated membrane protein LAMP-1 (FIGS. 7A-7J). SYNB1891 was actively internalized by BMDCs as pretreatment with cytochalasin D significantly reduced the number of bacterial cells found within BMDCs (FIG. 7C). SYNB1891 induced significant expression of IFNβ1 as compared to control EcN in a phagocytosis-dependent manner (FIGS. 7D-7J). Cytochalasin D did not affect IFNβ1 induction by a soluble smSTING agonist, 2′3′-c-di-AM(PS)2 (Rp,Rp), in target cells. Therefore, phagocytosis of SYNB1891 is required for intracellular CDA release and STING activation in target cells, providing a natural mechanism for preferential activation of APCs in the tumor. In addition to IFNβ1, SYNB1891, and control EcN, elicited secretion of other inflammatory cytokines such as IL-6 and TNFα (FIGS. 7H-7J), while exposure to smSTING led only to IFNβ1 expression. Inhibition of phagocytosis did not significantly impact TNFα production, yet decreased IL-6 expression by 2-3 fold (FIGS. 7H-7J), demonstrating that EcN can initiate immune signaling from the surface of the phagocytic cell, although internalization is required to optimally enhance signaling.

Type I interferon production by BMDCs in response to SYNB1891 was significantly dependent on STING signaling, as STING BMDCs failed to induce high levels of IFNβ1 expression (FIGS. 7H-7J). EcN chassis itself induced intermediate levels of IFNβ1 expression in a STING-independent but TLR4-dependent manner, possibly through LPS/TLR4 activation. IFNβ1 expression in response to smSTING agonist was preserved in TLR4^{-/-} BMDCs and completely abrogated in BMDCs (FIGS. 7H-7J). LPS is an integral component of the outer membrane of Gram-negative bacteria and as such LPS recognition by TLR4 plays an important role in the induction of immune responses by these microbes. Indeed, lack of TLR4 signaling significantly blunted expression of inflammatory cytokines such as IL-6 and IL-12p35 by BMDCs in response to SYNB1891 and control EcN (FIGS. 7H-7J), suggesting that LPS is an important component of the immunostimulatory mechanism of SYNB1891.

Collectively, these data demonstrate that SYNB1891 phagocytosis by APCs is required for STING-dependent induction of type I IFN responses. Moreover, the EcN chassis of SYNB1891 activates parallel TLR4-dependent signaling that results in the expression of additional pro-inflammatory cytokines which are amplified by phagocytosis of the therapeutic.

### Example 5

### B-16 Tumor Model

To determine in vivo activity and efficacy of various bacteria in combination with an immune modulator, e.g., an immune initiator or an immune sustainer, over time, the B-16 tumor model is used.

B16 tumors are injected into mice (2×10⁵/mouse/40-80 mL in PBS) then injected intratumorally with three different doses of bacteria and/or immune modulator(s) every three days for one week. On day 0, mice are randomized into groups (N=16 per group) for intratumor dosing as follows: PBS (group 1, vehicle control), wild-type bacteria, wild-type bacteria plus immune modulator, bacterial chassis, and bacterial chassis plus immune modulator. Tumor toxicity (weight) and growth are measured at various time points, while tumors are weighed and processed at the conclusion of the experiment.

The results will demonstrate that wild-type bacteria and bacterial chassis, administered in combination with an immune modulator, are able to provide an anti-tumoral response.

### Example 6

### SYNB1891 Generates Efficacious Anti-Tumor Immunity

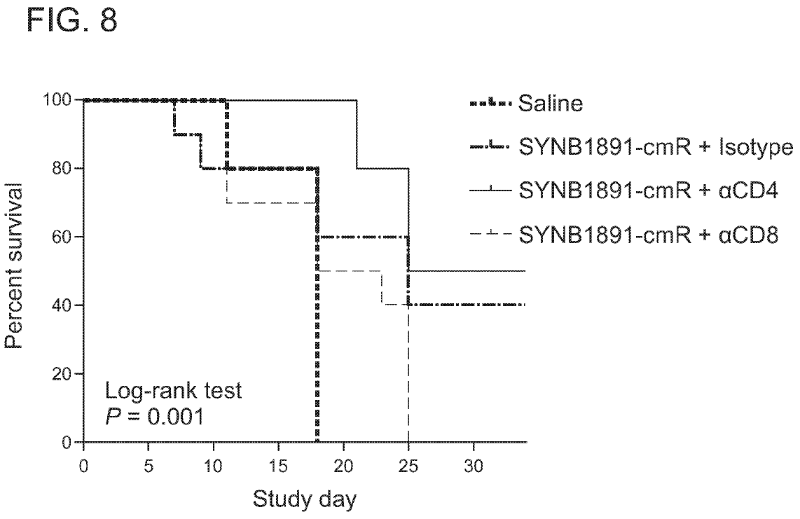

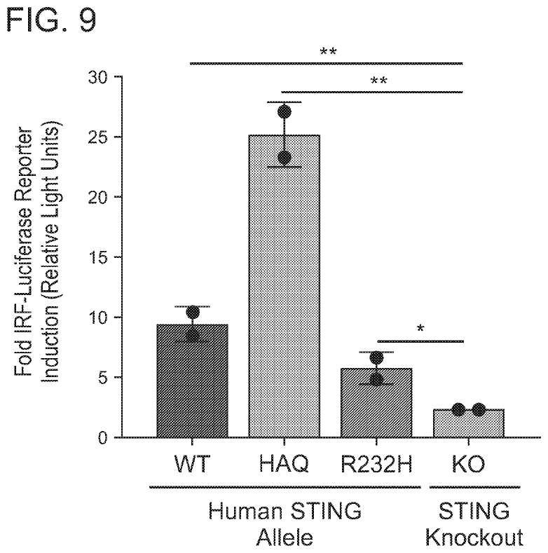

To evaluate the contribution of T cells towards SYNB1891 efficacy in the A20 tumor model, CD4+ T cells or CD8+ T cells were depleted prior-to treatment initiation and throughout the course of the study using depleting antibodies. While mice treated with either isotype control or a CD4+ T cell-depleting antibody exhibited a 40-50% complete response rate, 0% of mice receiving a CD8+ T cell-depleting antibody survived long-term (FIG. 8). These data demonstrate that CD8+ T cells critically contribute to the long-term efficacy of SYNB1891.

### Example 7

### SYNB1891 Activates Multiple STING Alleles in Human Antigen Presenting Cells

The activity of SYNB1891 amongst a panel of human monocyte (THP-1) IRF reporter cell lines which contained three of the most prevalent TMEM173 (STING) variants was evaluated. WT represents 57.9%, HAQ represents 20.4% and R232H represents 13.7% of alleles found in the human population, respectively. SYNB1891 treatment resulted in type I IFN pathway induction for all three alleles (FIG. 9). The HAQ allele showed the highest level of induction with the WT and R232H alleles showing intermediate and lower activity, respectively. Activity in the THP-1 cells was STING-dependent, as reporter signal was significantly attenuated in STING^{-/-} cells (FIG. 9).

1. A pharmaceutical composition comprising an isolated bacterium, at least one immune modulator, and a pharmaceutically acceptable carrier, wherein the bacterium is a wild-type bacterium or a bacterial chassis.

2. The composition of claim 1, wherein the at least one immune modulator is at least one immune initiator.

3. The composition of claim 2, wherein the immune initiator is capable of enhancing oncolysis, activating antigen presenting cells (APCs), and/or priming and activating T cells.

4. The composition of any one of claims 2-3, wherein the immune initiator is a STING agonist, arginine, 5-FU, TNFα, IFNγ, IFNβ1, agonistic anti-CD40 antibody, CD40L, SIRPα, GMCSF, agonistic anti-OXO40 antibody, OXO40L, agonistic anti-4-1BB antibody, 4-1BBL, agonistic anti-GITR antibody, GITRL, anti-PD1 antibody, anti-PDL1 antibody, or azurin.

5. The composition of claim 4, wherein the STING agonist is c-diAMP, c-GAMP, or c-diGMP.

6. The composition of any one of claims 2-3, wherein the immune initiator is a cytokine, a chemokine, a single chain antibody, a ligand, a metabolic converter, a T cell co-stimulatory receptor, a T cell co-stimulatory receptor ligand, an engineered chemotherapy, or a lytic peptide.

7. The composition of claim 2, wherein the immune initiator is arginine

8. The composition of claim 2, wherein the immune initiator is 5-FU.

9. The composition of claim 1, wherein the at least one immune modulator is at least one immune sustainer.

10. The composition of claim 9, wherein the immune sustainer is capable of enhancing trafficking and infiltration of T cells, enhancing recognition of cancer cells by T cells, enhancing effector T cell response, and/or overcoming immune suppression.

11. The composition of claim 9 or claim 10, wherein the immune sustainer is a metabolic converter, arginine, a STING agonist, CXCL9, CXCL10, anti-PD1 antibody, anti-PDL1 antibody, anti-CTLA4 antibody, agonistic anti-GITR antibody or GITRL, agonistic anti-OX40 antibody or OX40L, agonistic anti-4-1BB antibody or 4-1BBL, IL-15, IL-15 sushi, IFNγ, or IL-12.

12. The composition of any one of claims 9-11, wherein the immune sustainer is a cytokine, a chemokine, a single chain antibody, a ligand, a metabolic converter, a T cell co-stimulatory receptor, or a T cell co-stimulatory receptor ligand.

13. The composition of claim 9 or claim 10, wherein the at least one immune sustainer is a kynureninase.

14. The composition of claim 9 or claim 10, wherein the immune sustainer is arginine

15. The composition of any one of claims 9-11, wherein the immune sustainer is a STING agonist.

16. The composition of claim 15, wherein the STING agonist is c-diAMP, c-GAMP, or c-diGMP.

17. The composition of any one of the previous claims, wherein the at least one immune modulator comprises at least one immune initiator and at least one immune sustainer.

18. The composition of any one of the previous claims, wherein the at least one immune modulator is not produced by the bacterium.

19. The composition of any one of the previous claims, wherein the bacterium is a wild-type E. coli Nissle bacterium.

20. The composition of any one of claims 1-18, wherein the bacterial chassis is a bacterium comprising at least one mutation or deletion in a gene which results in one or more auxotrophies.

21. The composition of claim 20, wherein the bacterial chassis is a bacterium comprising a thyA auxotrophy and/or a dapA auxotrophy.

22. The composition of claim 20 or claim 21, wherein the bacterial chassis is an E. coli, Lactobacillus, Lactococcus, Salmonella, Listeria, Lactobacillus, Lactococcus, Bifido bacterium, C. novyi, Streptococcus pyogenes, Myco bovis, or Klebsiella bacterium.

23. The composition of any one of claims 20-22, wherein the bacterial chassis further comprises a phage deletion.

24. The composition of any one of the previous claims, wherein the composition is formulated for intratumoral administration.

25. A syringe comprising the composition of any one of the previous claims.

26. A kit comprising the composition of any one of claims 1-24 or the syringe of claim 25, and instructions for use thereof.

27. A kit comprising:
i) a first composition comprising an isolated bacterium, wherein the bacterium is a wild-type bacterium or a bacterial chassis,
ii) a second composition comprising an immune modulator, and
iii) instructions for use thereof.

28. The kit of claim 27, wherein the first composition is a lyophilized composition.

29. The kit of claim 27, wherein the instructions for use indicate that
the first composition is for administration to a subject prior to the second composition;
the second composition is for administration to a subject prior to the first composition; or
the first and second compositions are combined before administration to a subject.

30. A method of treating cancer in a subject, the method comprising administering to the subject the composition of any one of claims 1-24, thereby treating cancer in the subject.

31. A method of inducing and sustaining an immune response in a subject, the method comprising administering to the subject the composition of any one of claims 1-24, thereby inducing and sustaining the immune response in the subject.

32. The method of claim 31, wherein the bacterium undergoes phagocytosis following administration to the subject.

33. The method of claim 31, wherein the induction of the immune response is synergistically enhanced by phagocytosis of the bacterium.

34. A method of inducing an abscopal effect in a subject having a tumor, the method comprising administering to the subject the composition of any one of claims 1-24, thereby inducing the abscopal effect in the subject.

35. A method of inducing immunological memory in a subject having a tumor, the method comprising administering to the subject the composition of any one of claims 1-24, thereby inducing the immunological memory in the subject.

36. A method of inducing partial regression of a tumor in a subject, the method comprising administering to the subject the composition of any one of claims 1-24, thereby inducing the partial regression of the tumor in the subject.

37. The method of claim 36, wherein the partial regression is a decrease in size of the tumor by at least about 10%, at least about 25%, at least about 50%, or at least about 75%.

38. A method of inducing complete regression of a tumor in a subject, the method comprising administering to the subject the composition of any one of claims 1-24, thereby inducing the complete regression of the tumor in the subject.

39. The method of claim 38, wherein the tumor is not detectable in the subject after administration of the pharmaceutically acceptable composition.

40. The method of any one of claims 30-39, wherein the administering is intratumoral injection.

41. A method of treating cancer in a subject, the method comprising
administering a bacterium to the subject, wherein the bacterium is a wild-type bacterium or a bacterial chassis; and
administering at least one immune modulator to the subject,
thereby treating cancer in the subject.

42. A method of inducing and sustaining an immune response in a subject, the method comprising
administering a bacterium to the subject, wherein the bacterium is a wild-type bacterium or a bacterial chassis; and
administering at least one immune modulator to the subject,
thereby inducing and sustaining the immune response in the subject.

43. The method of claim 39 or claim 40,
wherein the administering steps are performed at the same time;
wherein administering of the bacterium to the subject occurs before administering of the at least one immune modulator to the subject; or
wherein administering of the at least one immune modulator to the subject occurs before administering of the bacterium to the subject.

44. The method of any one of claims 30-43, further comprising a step of selecting a subject who would benefit from treatment with the bacterium and the at least one immune modulator.

45. The method of any one of claims 30-44, wherein the bacterium colonizes a tumor in the subject.

46. The method of any one of claims 39-45, wherein the administering of the bacterium is intratumoral injection.

47. The method of any one of claims 30-46, wherein the administering is not oral administration.

48. The method of any one of claims 39-45, wherein the administering of the at least one immune modulator is an intravenous injection or intrathecal injection.

49. The method of any one of claims 30-48, wherein the bacterium comprise a homogenous population of predefined bacteria.

50. The method of claim 49, wherein the homogenous population of predefined bacteria comprise E. coli Nissle.

51. The method of any one of claims 39-50, wherein the at least one immune modulator comprises at least one immune initiator and at least one immune sustainer.

52. The method of claim 51, wherein the at least one immune initiator is selected from the immune initiators listed in Table 5, and the at least one immune sustainer is selected from the immune sustainers listed in Table 6.

53. The method of claim 52, wherein the at least one immune initiator is a STING agonist, and the at least one immune sustainer is a kynureninase.

54. A method of activating antigen presenting cells in a tumor in a subject, the method comprising administering a bacterium to the subject, wherein the bacterium is a bacterial chassis or a bacterium producing a STING agonist, wherein the bacterium undergoes phagocytosis after administration in the subject, thereby activating antigen presenting cells in the tumor in the subject.

55. The method of any one of claims 30-54, wherein the method further comprises
selecting a subject having at least one wild-type STING allele, HAQ STING allele, or R232H STING allele.
